SEQUENCE LISTING (87 sequences)

SEQ ID NO: 1 - DNA, 342 bp, Artificial Sequence
  Synthetic nucleic aicd
gaggttcagctgcagcagtctggggctgagcttgtgaggccaggggcctcagtcaagttgtcctgcacagcttctggctttaacattaaagacgactatatgcactgggtgaagcagaggcctgaacagggcctggagtggattggatggattgatcctgagaatggtgatactgaatatgcctcgaagttccagggcaaggccactataacagcagacacatcctccaacacagcctacctgcagctcagcagcctgacatctgaggacactgccgtctattactgtactcgtagctccgtaggctactggggccaaggcaccactctcacagtctcctca

SEQ ID NO: 2 - protein, 114 aa, Artificial Sequence
  Synthetic polypeptide
EVQLQQSGAELVRPGASVKLSCTASGFNIKDDYMHWVKQRPEQGLEWIGWIDPENGDTEYASKFQGKATITADTSSNTAYLQLSSLTSEDTAVYYCTRSSVGYWGQGTTLTVSS

SEQ ID NO: 3 - DNA, 336 bp, Artificial Sequence
  Synthetic nucleic acid
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacttatttcacactcaagatcagcagagtggaggctgaggatctgggagtttatttctgctctcaaagaacacatgttccgtacacgttcggaggggggaccaagctggagataaaa

SEQ ID NO: 4 - protein, 112 aa, Artificial Sequence
  Synthetic polypeptide
DVVMTQTPLSLPVSLGDQASISCRSSQSLVHSNGNTYLHWYLQKPGQSPKLLIYKVSNRFSGVPDRFSGSGSGTYFTLKISRVEAEDLGVYFCSQRTHVPYTFGGGTKLEIK

SEQ ID NO: 5 - DNA, 357 bp, Artificial Sequence
  Synthetic nucleic acid
caggtgcagctggtggagtctgggggaggcttggtgcagcccggggggtctctgagactctcctgtgtagcctctggatacagctacagtattggttacatggcctggttccgccaggccccaggaaaggagcgcgcgtgggtcgcgtctcgatatactggtgacggtggcgcagtctttgacgacgccgtgaagggccgattcaccacctcccaagagagtgccgggaacacgttcgatttgcaaatggacagcctgaaacctgaggacactgccatgtactattgcgcagcgaaagggcccggtttcgggcggtgggagtactggggccgggggacccaggtcaccgtctcctca

SEQ ID NO: 6 - protein, 119 aa, Artificial Sequence
  Synthetic polypeptide
QVQLVESGGGLVQPGGSLRLSCVASGYSYSIGYMAWFRQAPGKERAWVASRYTGDGGAVFDDAVKGRFTTSQESAGNTFDLQMDSLKPEDTAMYYCAAKGPGFGRWEYWGRGTQVTVSS

SEQ ID NO: 7 - protein, 12 aa, Homo sapiens
ESKYGPPCPPCP

SEQ ID NO: 8 - protein, 45 aa, Homo sapiens
TTTPAPRPPTPAPTIASQPLSLRPEACRPAAGGAVHTRGLDFACD

SEQ ID NO: 9 - protein, 119 aa, Homo sapiens
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 228 aa, Homo sapiens
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 21 aa, Homo sapiens
IYIWAPLAGTCGVLLLSLVIT

SEQ ID NO: 12 - protein, 27 aa, Homo sapiens
FWVLVVVGGVLACYSLLVTVAFIIFWV

SEQ ID NO: 13 - protein, 42 aa, Homo sapiens
KRGRKKLLYIFKQPFMRPVQTTQEEDGCSCRFPEEEEGGCEL

SEQ ID NO: 14 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 18 aa, Artificial Sequence
  Synthetic polypeptide
EGRGSLLTCGDVEENPGP

SEQ ID NO: 16 - protein, 22 aa, Homo sapiens
MLLLVTSLLLCELPHPAFLLIP

SEQ ID NO: 17 - protein, 335 aa, Homo sapiens
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 18 - protein, 862 aa, Artificial Sequence
  Synthetic protein
MLLLVTSLLLCELPHPAFLLIPHMEVQLQQSGAELVRPGASVKLSCTASGFNIKDDYMHWVKQRPEQGLEWIGWIDPENGDTEYASKFQGKATITADTSSNTAYLQLSSLTSEDTAVYYCTRSSVGYWGQGTTLTVSSGGGGSGGGGSGGGGSDVVMTQTPLSLPVSLGDQASISCRSSQSLVHSNGNTYLHWYLQKPGQSPKLLIYKVSNRFSGVPDRFSGSGSGTYFTLKISRVEAEDLGVYFCSQRTHVPYTFGGGTKLEIKTSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 19 - protein, 740 aa, Artificial Sequence
  Synthetic protein
MLLLVTSLLLCELPHPAFLLIPHMQVQLVESGGGLVQPGGSLRLSCVASGYSYSIGYMAWFRQAPGKERAWVASRYTGDGGAVFDDAVKGRFTTSQESAGNTFDLQMDSLKPEDTAMYYCAAKGPGFGRWEYWGRGTQVTVSSTSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 20 - protein, 707 aa, Artificial Sequence
  Synthetic protein
MLLLVTSLLLCELPHPAFLLIPHMQVQLVESGGGLVQPGGSLRLSCVASGYSYSIGYMAWFRQAPGKERAWVASRYTGDGGAVFDDAVKGRFTTSQESAGNTFDLQMDSLKPEDTAMYYCAAKGPGFGRWEYWGRGTQVTVSSTSESKYGPPCPPCPIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 21 - protein, 713 aa, Artificial Sequence
  Synthetic peptide
MLLLVTSLLLCELPHPAFLLIPHMQVQLVESGGGLVQPGGSLRLSCVASGYSYSIGYMAWFRQAPGKERAWVASRYTGDGGAVFDDAVKGRFTTSQESAGNTFDLQMDSLKPEDTAMYYCAAKGPGFGRWEYWGRGTQVTVSSTSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 22 - protein, 820 aa, Artificial Sequence
  Synthetic peptide
MLLLVTSLLLCELPHPAFLLIPHMQVQLVESGGGLVQPGGSLRLSCVASGYSYSIGYMAWFRQAPGKERAWVASRYTGDGGAVFDDAVKGRFTTSQESAGNTFDLQMDSLKPEDTAMYYCAAKGPGFGRWEYWGRGTQVTVSSTSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 23 - protein, 929 aa, Artificial Sequence
  Synthetic peptide
MLLLVTSLLLCELPHPAFLLIPHMQVQLVESGGGLVQPGGSLRLSCVASGYSYSIGYMAWFRQAPGKERAWVASRYTGDGGAVFDDAVKGRFTTSQESAGNTFDLQMDSLKPEDTAMYYCAAKGPGFGRWEYWGRGTQVTVSSTSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 24 - DNA, 20 bp, Artificial Sequence
  Synthetic oligonucleotide
gcagcgtgcacacgtggctg

SEQ ID NO: 25 - DNA, 22 bp, Artificial Sequence
  Synthetic oligonucleotide
ctggcccttacagtagccaggc

SEQ ID NO: 26 - DNA, 22 bp, Artificial Sequence
  Synthetic oligonucleotide
caccattggcaatgagcggttc

SEQ ID NO: 27 - DNA, 22 bp, Artificial Sequence
  Synthetic oligonucleotide
aggtctttgcggatgtccacgt

SEQ ID NO: 28 - DNA, 20 bp, Artificial Sequence
  Synthetic oligonucleotide
cctctcccgcggccgcctag

SEQ ID NO: 29 - DNA, 20 bp, Artificial Sequence
  Synthetic oligonucleotide
gagcgagcgttcggacctcg

SEQ ID NO: 30 - protein, 12 aa, Artificial Sequence
  Synthetic peptide
ESKYGPPSPPSP

SEQ ID NO: 31 - protein, 18 aa, Homo sapiens
DPASKSRSCGEVRQIYGA

SEQ ID NO: 32 - protein, 18 aa, Homo sapiens
GEVRQIYGAKGFSLSDVP

SEQ ID NO: 33 - protein, 18 aa, Homo sapiens
KGFSLSDVPQAEISGEHL

SEQ ID NO: 34 - protein, 18 aa, Homo sapiens
QAEISGEHLRICPQGYTC

SEQ ID NO: 35 - protein, 18 aa, Homo sapiens
RICPQGYTCCTSEMEENL

SEQ ID NO: 36 - protein, 18 aa, Homo sapiens
CTSEMEENLANRSHAELE

SEQ ID NO: 37 - protein, 18 aa, Homo sapiens
ANRSHAELETALRDSSRV

SEQ ID NO: 38 - protein, 18 aa, Homo sapiens
TALRDSSRVLQAMLATQL

SEQ ID NO: 39 - protein, 18 aa, Homo sapiens
LQAMLATQLRSFDDHFQH

SEQ ID NO: 40 - protein, 18 aa, Homo sapiens
RSFDDHFQHLLNDSERTL

SEQ ID NO: 41 - protein, 18 aa, Homo sapiens
LLNDSERTLQATFPGAFG

SEQ ID NO: 42 - protein, 18 aa, Homo sapiens
QATFPGAFGELYTQNARA

SEQ ID NO: 43 - protein, 18 aa, Homo sapiens
ELYTQNARAFRDLYSELR

SEQ ID NO: 44 - protein, 18 aa, Homo sapiens
FRDLYSELRLYYRGANLH

SEQ ID NO: 45 - protein, 18 aa, Homo sapiens
LYYRGANLHLEETLAEFW

SEQ ID NO: 46 - protein, 18 aa, Homo sapiens
LEETLAEFWARLLERLFK

SEQ ID NO: 47 - protein, 18 aa, Homo sapiens
ARLLERLFKQLHPQLLLP

SEQ ID NO: 48 - protein, 18 aa, Homo sapiens
QLHPQLLLPDDYLDCLGK

SEQ ID NO: 49 - protein, 18 aa, Homo sapiens
DDYLDCLGKQAEALRPFG

SEQ ID NO: 50 - protein, 18 aa, Homo sapiens
QAEALRPFGEAPRELRLR

SEQ ID NO: 51 - protein, 18 aa, Homo sapiens
EAPRELRLRATRAFVAAR

SEQ ID NO: 52 - protein, 18 aa, Homo sapiens
ATRAFVAARSFVQGLGVA

SEQ ID NO: 53 - protein, 18 aa, Homo sapiens
SFVQGLGVASDVVRKVAQ

SEQ ID NO: 54 - protein, 18 aa, Homo sapiens
SDVVRKVAQVPLGPECSR

SEQ ID NO: 55 - protein, 18 aa, Homo sapiens
VPLGPECSRAVMKLVYCA

SEQ ID NO: 56 - protein, 18 aa, Homo sapiens
AVMKLVYCAHCLGVPGAR

SEQ ID NO: 57 - protein, 18 aa, Homo sapiens
HCLGVPGARPCPDYCRNV

SEQ ID NO: 58 - protein, 18 aa, Homo sapiens
PCPDYCRNVLKGCLANQA

SEQ ID NO: 59 - protein, 18 aa, Homo sapiens
LKGCLANQADLDAEWRNL

SEQ ID NO: 60 - protein, 18 aa, Homo sapiens
DLDAEWRNLLDSMVLITD

SEQ ID NO: 61 - protein, 18 aa, Homo sapiens
LDSMVLITDKFWGTSGVE

SEQ ID NO: 62 - protein, 18 aa, Homo sapiens
KFWGTSGVESVIGSVHTW

SEQ ID NO: 63 - protein, 18 aa, Homo sapiens
SVIGSVHTWLAEAINALQ

SEQ ID NO: 64 - protein, 18 aa, Homo sapiens
LAEAINALQDNRDTLTAK

SEQ ID NO: 65 - protein, 18 aa, Homo sapiens
DNRDTLTAKVIQGCGNPK

SEQ ID NO: 66 - protein, 18 aa, Homo sapiens
VIQGCGNPKVNPQGPGPE

SEQ ID NO: 67 - protein, 18 aa, Homo sapiens
VNPQGPGPEEKRRRGKLA

SEQ ID NO: 68 - protein, 18 aa, Homo sapiens
EKRRRGKLAPRERPPSGT

SEQ ID NO: 69 - protein, 18 aa, Homo sapiens
PRERPPSGTLEKLVSEAK

SEQ ID NO: 70 - protein, 18 aa, Homo sapiens
LEKLVSEAKAQLRDVQDF

SEQ ID NO: 71 - protein, 18 aa, Homo sapiens
AQLRDVQDFWISLPGTLC

SEQ ID NO: 72 - protein, 18 aa, Homo sapiens
WISLPGTLCSEKMALSTA

SEQ ID NO: 73 - protein, 18 aa, Homo sapiens
SEKMALSTASDDRCWNGM

SEQ ID NO: 74 - protein, 18 aa, Homo sapiens
SDDRCWNGMARGRYLPEV

SEQ ID NO: 75 - protein, 18 aa, Homo sapiens
ARGRYLPEVMGDGLANQI

SEQ ID NO: 76 - protein, 18 aa, Homo sapiens
MGDGLANQINNPEVEVDI

SEQ ID NO: 77 - protein, 18 aa, Homo sapiens
NNPEVEVDITKPDMTIRQ

SEQ ID NO: 78 - protein, 18 aa, Homo sapiens
TKPDMTIRQQIMQLKIMT

SEQ ID NO: 79 - protein, 18 aa, Homo sapiens
QIMQLKIMTNRLRSAYNG

SEQ ID NO: 80 - protein, 18 aa, Homo sapiens
NRLRSAYNGNDVDFQDAS

SEQ ID NO: 81 - protein, 18 aa, Homo sapiens
NDVDFQDASDDGSGSGSG

SEQ ID NO: 82 - protein, 18 aa, Homo sapiens
DDGSGSGSGDGCLDDLCS

SEQ ID NO: 83 - protein, 18 aa, Homo sapiens
DGCLDDLCSRKVSRKSSS

SEQ ID NO: 84 - protein, 18 aa, Homo sapiens
RKVSRKSSSSRTPLTHAL

SEQ ID NO: 85 - protein, 18 aa, Homo sapiens
SRTPLTHALPGLSEQEGQ

SEQ ID NO: 86 - protein, 15 aa, Homo sapiens
PGLSEQEGQKTSAAS

SEQ ID NO: 87 - protein, 6 aa, Artificial Sequence
  Synthetic peptide
APEFLG

(19) United States
(12) Patent Application Publication
(10) Pub. No.: US 2023/0340146 A1
(43) Pub. Date: Oct. 26, 2023

(54) IGG4 HINGE-CONTAINING CHIMERIC ANTIGEN RECEPTORS TARGETING GLYPICAN-1 (GPC1) FOR TREATING SOLID TUMORS

(71) Applicant: The U.S.A, as represented by the Secretary, Department of Health and Human Services, Bethesda, MD (US)

(72) Inventors: Mitchell Ho, Urbana, MD (US); Nan Li, Laurel, MD (US); Jessica Diana Hong, Herndon, VA (US)

(73) Assignee: The U.S.A., as represented by the Secretary, Department of Health and Human Services, Bethesda, MD (US)

(21) Appl. No.: 18/020,191

(22) PCT Filed: Aug. 10, 2021
(86) PCT No.: PCT/US2021/045305

§ 371 Date: Feb. 7, 2023

Related U.S. Application Data
(60) Provisional application No. 63/065,388, filed on Aug. 13, 2020.

(51) Int. Cl. C07K 14/725; C12N 15/86; C07K 16/30; A61P 35/00

(52) U.S. Cl. CPC C07K 16/303; A61P 35/00; C07K 14/7051; C12N 15/86; C07K 2317/53; C07K 2317/565; C07K 2317/569; C07K 2317/622; C07K 2319/02; C07K 2319/03

(57) ABSTRACT

Optimized chimeric antigen receptors (CARs) targeting glypican-1 (GPC1) that include a 12-amino acid hinge region from IgG4 are described. The optimized CARs include a transmembrane domain from either CD8 or CD28. Immune cells, such as T cells or natural killer cells, expressing the optimized CARs can be used to treat GPC1-positive solid tumors.

### CROSS REFERENCE TO RELATED APPLICATIONS

This application claims of the benefit of U.S. Application No. 63/065,388, filed Aug. 13, 2020, which is herein incorporated by reference in its entirety.

### FIELD

This disclosure concerns optimized chimeric antigen receptors (CARs) specific for tumor antigen glypican 1 (GPC1) that include a hinge region from IgG4. This disclosure further concerns use of the GPC1-targeted IgG4 hinge-containing CARs for treating solid tumors.

### ACKNOWLEDGMENT OF GOVERNMENT SUPPORT

This invention was made with government support under Project No. Z01 BC010891 awarded by the National Institutes of Health. The government has certain rights in the invention.

### BACKGROUND

Remarkable responses following CD19 chimeric antigen receptor (CAR) T-cell therapy in patients with relapsed and refractory B cell malignancies has led to the approval of two CD19 CAR T cell products by the Food and Drug Administration (FDA) (Porter et al., N Engl J Med 2011;365:725-733; Kochenderfer et al., Blood 2012;119:2709-2720). However, emerging follow-up data demonstrates that only 30% to 50% of patients experience long-term disease control (Maude et al., N Engl J Med 2018;378:439-448; Park et al., N Engl J Med 2018;378:449-459). To improve the response rate in B cell malignancies and translate the success of CAR T cells to solid tumors, the optimization of this class of therapeutics is required. It has been proposed that the length of the hinge (also referred as the spacer) is important for providing adequate intracellular distance for immunological synapse formation (Srivastava et al., Trends Immunol 2015;36:494-502). The spacer also provides flexibility to access the targeted antigens (Guest et al., J Immunother 2005;28:203-211). Tailoring the spacer region from the modified IgG4 hinge and Fc domains has been found to improve the antitumor efficacy of CAR T cells in vivo (Hudecek et al., Clin Cancer Res 2013;19:3153-3164; Hudecek et al., Cancer Immunol Res 2015;3:125-135; Jonnalagadda et al., Mol Ther 2015;23:757-768).

Glypican 1 (GPC1) is a glycosylphosphatidylinositol-anchored cell surface protein. It is mainly expressed in the neural and skeletal systems during embryonic development and is expressed at low levels in adult tissues (Awad et al., Atlas Genet Cytogenet Oncol Haematol 2014;18:461-464). GPC1 expression is elevated in pancreatic cancer, both by the cancer cells and the adjacent fibroblasts, whereas its expression is rarely found in normal pancreas (Duan et al., Asian J Surg 2013;36:7-12; Kleeff et al., J Clin Invest 1998;102:1662-1673). Two anti-GPC1 monoclonal antibodies, clone 01a033 and clone 1-12, have been utilized to develop an antibody-drug-conjugate (ADC) and CAR T cells against GPC1-positive tumor cells and were found to possess antitumor efficacy in preclinical models (Harada et al., Oncotarget 2017;8:24741-24752; Kato et al., Int J Cancer 2018;142:1056-1066; Matsuzaki et al., Int J Cancer 2018;142:1056-1066; Nishigaki et al., Br J Cancer 2020;122:1333-1341).

Attention has been drawn to the various expression levels of GPC1 in pancreatic cancer. A study showed that 51.4% had weak, 35.1% had moderate, and 13.5% had strong staining of GPC1 in the positively stained pancreatic tumor tissues (n=111) (Lu et al., Cancer Med 2017;6:1181-1191). Antigen density has emerged as a major factor influencing the activity of CAR T cells (Majzner and Mackall, Cancer Discov 2018;8:1219-1226; Shah and Fry, Nat Rev Clin Oncol 2019;16:372-385). CAR T cell potency is highly dependent on target antigen expression, and CARs often fail to exert their antitumor activities when antigen expression is low or below a certain threshold. Hinge and transmembrane (TM) changes in CAR design can tune the threshold of antigen density required for optimal CAR T cell activity (Majzner et al., Cancer Discov 2020;10:702-723).

### SUMMARY

Described herein is the development of optimized GPC1-specific chimeric antigen receptors (CARs) having particular hinge and transmembrane regions. In some cases, the CARs are comprised of an antibody (or antigen-binding fragment thereof) that has high affinity for either the N-lobe (membrane distal) or C-lobe (membrane proximal) of GPC1. It is disclosed herein that the hinge and transmembrane domain of the CARs exert a major effect on T cell function, particularly when GPC1 density on GPC1-expressing cancer cells is low.

Provided herein is a CAR that includes an extracellular antigen-binding domain specific for GPC1; an IgG4 hinge sequence; a transmembrane domain; an intracellular co-stimulatory domain; and an intracellular signaling domain. In some embodiments, the CAR includes a hinge region consisting of the IgG4 hinge region set forth as SEQ ID NO: 7. In some embodiments, the antigen-binding domain of the CAR specifically binds a membrane distal epitope of GPC1. In some examples, the antigen-binding domain includes the CDR sequences of GPC1-specific single-domain antibody D4 or the VH and VL CDR sequences of GPC1-specific antibody HM2. In some examples, the transmembrane domain of the CAR is a CD28 transmembrane domain. In other examples, the transmembrane domain of the CAR is a CD8α transmembrane domain.

Nucleic acid molecules encoding a disclosed CAR are further provided. In some embodiments, the nucleic acid molecule includes in the 5′ to 3′ direction a nucleic acid encoding a first granulocyte-macrophage colony stimulating factor receptor signal sequence (GMCSFRss); a nucleic acid encoding the antigen-binding domain; a nucleic acid encoding the IgG4 hinge region; a nucleic acid encoding the transmembrane domain; a nucleic acid encoding the co-stimulatory domain; a nucleic acid encoding the signaling domain; a nucleic acid encoding a self-cleaving 2A peptide; a nucleic acid encoding a second GMCSFRss; and a nucleic acid encoding a truncated human epidermal growth factor receptor (huEGFRt). In some examples, the nucleic acid molecule further includes a human elongation factor 1α (EF1α) promoter sequence 5′ of the nucleic acid encoding the first GMCSFRss. Vectors that include the disclosed nucleic acid molecules are further provided.

Also provided are isolated immune cells, such as T cells, NK cells or macrophages, expressing a CAR disclosed herein and/or containing an isolated nucleic acid molecule or vector disclosed herein.

Further provided are compositions that include a pharmaceutically acceptable carrier and a CAR, nucleic acid molecule, vector or cell disclosed herein.

Methods of treating a GPC1-positive cancer, or inhibiting tumor growth or metastasis of a GPC1-positive cancer, in a subject are also provided. In some embodiments, the methods include administering to the subject a therapeutically effective amount of a CAR, nucleic acid molecule, vector, cell or composition disclosed herein. In some examples, the GPC1-positive cancer is a solid tumor. In some examples, the GPC1-positive cancer is a tumor with a low density (such as low expression) of GPC-1, for example a tumor that expresses less than 2500 molecules of GPC1 per cell.

The foregoing and other objects and features of the disclosure will become more apparent from the following detailed description, which proceeds with reference to the accompanying figures.

### BRIEF DESCRIPTION OF THE DRAWINGS

FIGS. 1A-1G: Isolation of GPC1-specific antibodies using hybridoma technology and phage display technology. (FIG. 1A) Six mouse mAbs (HM1 to HM6) from three parental clones bound to human GPC1, but not to other human glypican members by ELISA (antibody concentration of 1 µg/ml). (FIG. 1B) Flow cytometry comparing the six mouse mAbs at a concentration of 10 µg/ml showed increased binding to GPC1-positive T3M4 pancreatic cancer cells compared with non-specific control IgG. (FIG. 1C) Polyclonal phage ELISA from the output phage of each round of panning. (FIG. 1D) Monoclonal phage ELISA analysis of reactivity the D4 antibody to human and mouse GPC1 and other human glypican members. (FIG. 1E) Octet kinetic analysis for the interaction between HM2 and human GPC1. The K_{D} value was 0.4 nM. (FIG. 1F) Octet kinetic analysis for the interaction between D4 and human GPC1. The K_{D} value was 0.7 nM. (FIG. 1G) Cell-surface GPC1 expression in GPC1-negative A431 cells, GPC1-overexpressing H8 and 2B9 cells, as well as GPC1-positive pancreatic cancer T3M4 and KLM1 cells. Peaks represent the cell surface staining of GPC1 using 10 µg/ml HM2, D4 or isotype control. Data are represented as mean ± SEM of three independent experiments.

FIGS. 2A-2D: Increased expression of GPC1 in pancreatic cancer. (FIG. 2A) GPC1 mRNA levels are increased in the majority of pancreatic cancer cell lines including Miapaca-2, Panc-1, Aspc-1, Bxpc3, T3M4, Colo357, KLM1 and SU8686 compared with normal pancreatic duct cell line hTERT-HPNE. (FIG. 2B) GPC1 protein levels are also elevated in pancreatic cancer cell lines including T3M4, KLM1, Miapaca-2, SU8686, Bxpc3 and Panc-1 compared with normal pancreatic duct cell line hTERT-HPNE. HM2 antibody was used to detect GPC1 protein in the western blot. (FIG. 2C) GPC1 expression is detected in pancreatic tumor tissues at modest (ii) to high (iii) levels as compared to normal pancreas (i). (FIG. 2D) GPC1 expression is detected in NATs. 1 µg/ml of HM2 was used for IHC.

FIGS. 3A-3E: GPC1-targeted CAR T cells kill GPC1-positive tumor cells in vitro. (FIG. 3A) Schematic of a CAR construct. (FIG. 3B) Cytolytic activity of HM2 CAR T cells and mock T cells from 5 healthy donors after 24 hours of co-culture with 2B9 tumor cells. (FIG. 3C) CAR expression on T cells analyzed using flow cytometry by detection of hEGFRt expression. (FIG. 3D) Both HM2 and D4 CAR T cells potently lysed GPC1-positive H8, 2B9 and T3M4 cells without affecting GPC1-negative A431 cells after 24 hours of co-culture. (FIG. 3E) The above culture supernatants at the E:T ratio of 6.25:1 were harvested to measure IFN-y, IL-2 and TNF-α secretions via ELISA. Values represent mean ± SEM.

FIGS. 4A-4H: GPC1-targeted CAR T cells eradicate tumors in the 2B9 peritoneal dissemination xenograft mouse model. (FIG. 4A) Experimental schematic. 2B9 tumor-bearing NSG mice were treated with an i.p. injection of 10 million mock T cells, HM2 CAR T cells or D4 CAR T cells at day 11 after tumor cell inoculation. (FIG. 4B) HM2 and D4 CAR T cells regressed established Hep3B xenografts in 4 of 5 mice in each group. (FIG. 4C) Tumor bioluminescence as photons per second in mice treated in FIG. 4B. (FIG. 4D) Detection of CAR vector-positive cells in mouse spleen after 5 weeks of treatment. (FIG. 4E) Detection of CAR vector-positive cells in xenograft tumor tissues after 5 weeks of treatment. (FIG. 4F) Detection of CAR vector-positive cells in mouse pancreas after 5 weeks of treatment. (FIG. 4G) Distribution of integration sites in mice treated with HM2 and D4 CAR T cells. The integrated genes were largely shared in T cells recovered from various tissues of the same mouse, while some overlap was also observed in different mice receiving treatment. No integrated sites were found in mice that failed the D4 CAR T cell treatment. (FIG. 4H) The heatmap of shared integrated genes in the D4 and HM2 CAR groups. Values represent mean ± SEM.

FIGS. 5A-5H: D4 CAR T cells with a shorter spacer domain significantly improve its reactivity against low GPC1-expressing tumor cells. (FIG. 5A) Schematic of D4 CAR constructs. CD8TM was replaced with CD28TM in the original D4-CD8 hinge CAR. A shorter IgG4 hinge (12 aa) was used to replace the original CD8 hinge (45 aa). Either CD8 TM or CD28 TM was incorporated in the D4-IgG4 hinge-based CARs. (FIG. 5B) Transduction efficiency of the four D4 CAR constructs. (FIG. 5C) The D4-IgG4 hinge CD28 TM CAR T cells showed the best cytolytic activity among the four D4 CAR constructs when co-cultured with low GPC1-expressing T3M4 cells for 24 hours. (FIGS. 5D-5F) The D4-IgG4 hinge-CD28TM CAR T cells induced the greatest secretion of IFN-γ (FIG. 5D), CXCL10 (FIG. 5D), IL-2 (FIG. 5E), TNF-α (FIG. 5E) and IL-17A (FIG. 5F) upon stimulation with T3M4 cells at the E:T ratio of 6.25:1. (FIG. 5G) The D4-IgG4 hinge CAR T cells lost the enhanced reactivity when both cysteine residues were mutated. (FIG. 5H) Measurement of IFN-γ secretion in co-cultured supernatants at the E:T ratio of 6.25:1 as shown in FIG. 6F.

FIGS. 6A-6H: D4 CAR T cells with a short IgG4 spacer retain optimal reactivity compared with modified longer spacers. (FIG. 6A) Schematic of D4-IgG4 hinge-based CAR with different length of spacers (CH3 or CH2CH3). CD28 TM was used in all D4-IgG4 hinge-based CARs. (FIG. 6B) Transduction efficiency of the D4 CAR constructs shown in FIG. 6A. (FIG. 6C) The D4-IgG4 hinge CAR T cells showed the best cytolytic activity among the different D4 CAR constructs when co-cultured with low GPC1-expressing T3M4 cells for 24 hours. The enhanced reactivities in the three D4 CAR constructs were not observed in high GPC1-expressing 2B9 cells. Minimal cell lysis was found in A431 cells. (FIG. 6D) Measurement of IFN-γ secretion in co-cultured supernatants at the E:T ratio of 6.25:1. (FIG. 6E) Experimental schematic. T3M4 tumor-bearing NSG mice were i.p. injected with 10 million mock T cells, original D4-CD8 hinge-CD8TM CAR T cells, D4-IgG4 hinge CAR T cells, D4-IgG4 hinge-CH3 CAR T cells, or D4-IgG4 hinge-CH2CH3 CAR T cells at day 8 after tumor cell inoculation. (FIG. 6F) D4-IgG4 hinge-based CAR T cells rapidly eliminated T3M4 tumor cells in mice, while constructs with intermediate or long spacers only controlled tumor growth. (FIG. 6G) Tumor bioluminescence as photons per second in mice treated in FIG. 6F. (FIG. 6H) Kaplan-Meier survival curve reveals a significant extended survival of mice that received D4-IgG4 hinge CAR T cells.

FIGS. 7A-7C: Characterization and binding epitope of the HM2 and D4 antibodies. (FIG. 7A) HM2 specifically recognizes a binding epitope in peptide 53 by ELISA (#52, SEQ ID NO: 82; #53, SEQ ID NO: 83; #54, SEQ ID NO: 84). (FIG. 7B) D4 reacts to an epitope comprising both peptides 14 and 15 (#13, SEQ ID NO: 43; #14, SEQ ID NO: 44; #15, SEQ ID NO: 45). (FIG. 7C) Enlarged views of a 2D class average of GPC1 in complex with HM2 Fab and GPC1 in complex with D4-LR.

FIGS. 8A-8B: GPC1 expression in pancreatic cancer specimen as determined by immunohistochemistry. (FIG. 8A) The tissues were labeled with 1 µg/ml HM2 antibody. Images were obtained under 20X magnification. (FIG. 8B) Detailed information for each tissue specimen shown in FIG. 8A.

FIGS. 9A-9C: The tonic signaling of D4 CAR T cells with different hinges and transmembrane (TM) domains during ex vivo expansion. (FIGS. 9A-9B) Expression of T cell activation marker CD25 and exhaustion markers including PD1, TIM3 and LAG3 after initial activation in CD4+ (FIG. 9A) and CD8+ (FIG. 9B) CAR T cell populations. (FIG. 9C) percentage of activation marker and exhaustion markers in CD4+ and CD8+ CAR T cell populations based on FIGS. 9A and 9B.

FIG. 10: Memory T cell subsets of mock T cells and D4 CAR T cells with different hinges and TM domains. Shown is the relative proportion of stem cell-like memory (TSCM), central memory (TCM), effector memory (TEM), and terminally differentiated effector memory (TEMRA) subsets defined by CD62L, CD45RA and CD95 expression in the CD4+ and CD8+ CAR T cell population.

FIGS. 11A-11B: The cytolytic activity of D4 CAR T cells against GPC1 knockout (KO)-T3M4 cells. (FIG. 11A) None of the D4 CAR T cells with various hinges and TM domains lysed GPC1 KO-T3M4 cells after 24 hours of co-culture. (FIG. 11B) Minimal cell lysis was observed in D4-IgG4 hinge-CD28TM CAR T cells with or without cysteine mutations after stimulation with antigen negative cells (GPC1 KO-T3M4).

FIG. 12: Secretion of cytokines and chemokines by D4 CAR T cells with different hinges and TM domains upon stimulation by GPC1-positive T3M4 and GPC1 KO-T3M4 cells.

FIGS. 13A-13C: Incorporation of IgG4 hinge and CD28TM significantly improve antitumor efficacy. (FIG. 13A) Experimental schematic. T3M4 tumor-bearing NSG mice were i.p. injected with 5 million CD19 CAR T cells, D4-CD8 hinge-CD8 TM CAR T cells, D4-IgG4 hinge-CD8 TM CAR T cells or D4-IgG4 hinge-CD28 TM CAR T cells at day 8 after tumor cell inoculation. (FIG. 13B) D4-IgG4 hinge-based CAR T cells regressed T3M4 xenograft tumor growth, whereas the original D4-CD8 hinge-based CAR failed to control tumor growth. D4-IgG4 hinge-CD28 TM CAR showed better efficacy than D4-IgG4 hinge-CD8 TM CAR. (FIG. 13C) Tumor bioluminescence as photons per second in mice treated in FIG. 13B.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Aug. 4, 2021, 68.6 KB, which is incorporated by reference herein. In the accompanying sequence listing:

SEQ ID NO: 1 is the nucleotide sequence of the VH domain of the HM2 antibody.

SEQ ID NO: 2 is the amino acid sequence of the VH domain of the HM2 antibody.

SEQ ID NO: 3 is the nucleotide sequence of the VL domain of the HM2 antibody.

SEQ ID NO: 4 is the amino acid sequence of the VL domain of the HM2 antibody.

SEQ ID NO: 5 is the nucleotide sequence of the D4 antibody.

SEQ ID NO: 6 is the amino acid sequence of the D4 antibody.

SEQ ID NO: 7 is the amino acid sequence of an IgG4 hinge region.

SEQ ID NO: 8 is the amino acid sequence of a CD8α hinge region.

SEQ ID NO: 9 is the amino acid sequence of an IgG4-CH2 hinge region.

SEQ ID NO: 10 is the amino acid sequence of an IgG4-CH2-CH3 hinge region.

SEQ ID NO: 11 is the amino acid sequence of a CD8α transmembrane domain.

SEQ ID NO: 12 is the amino acid sequence of a CD28 transmembrane domain

SEQ ID NO: 13 is the amino acid sequence of a 4-1BB signaling moiety.

SEQ ID NO: 14 is the amino acid sequence of a CD3ζ signaling domain.

SEQ ID NO: 15 is the amino acid sequence of a self-cleaving T2A peptide.

SEQ ID NO: 16 is the amino acid sequence of a GMCSFRss.

SEQ ID NO: 17 is the amino acid sequence of huEGFRt.

SEQ ID NO: 18 is the amino acid sequence of the HM2-CD8 hinge-CD8 TM CAR.

SEQ ID NO: 19 is the amino acid sequence of the D4-CD8 hinge-CD8 TM CAR.

SEQ ID NO: 20 is the amino acid sequence of the D4-IgG4 hinge-CD8 TM CAR.

SEQ ID NO: 21 is the amino acid sequence of the D4-IgG4 hinge-CD28 TM CAR.

SEQ ID NO: 22 is the amino acid sequence of the D4-IgG4 hinge-CH3-CD28 TM CAR.

SEQ ID NO: 23 is the amino acid sequence of the D4-IgG4 hinge-CH2CH3-CD28 TM
CAR.

SEQ ID NOs: 24-27 are primer sequences.

SEQ ID NOs: 28 and 29 are sgRNA sequences.

SEQ ID NO: 30 is the amino acid sequence of a
modified IgG4 hinge region.

SEQ ID NOs: 31-86 are amino acid sequences of GPC1 peptides.

SEQ ID NO: 87 is the amino acid sequence of a peptide.

### DETAILED DESCRIPTION

I

Abbreviations

| | |
|---|---|
| CAR | chimeric antigen receptor |
| CDR | complementarity determining region |
| CTL | cytotoxic T lymphocyte |
| EF1α | elongation factor 1 alpha |
| EGF | epidermal growth factor |
| EGFR | epidermal growth factor receptor |
| ELISA | enzyme-linked immunosorbent assay |
| GPC1 | glypican-1 |
| GMCSFRss | granulocyte-macrophage colony stimulating factor receptor signal sequence |
| huEGFRt | human truncated epidermal growth factor receptor |
| IFN | interferon |
| Ig | immunoglobulin |
| IL | interleukin |
| i.p. | intraperitoneal |
| NAT | normal tissue adjacent to a tumor |
| TM | transmembrane |
| VH | variable heavy |
| VL | variable light |

### II. Summary of Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. All publications, patent applications, patents, and other references mentioned herein are incorporated by reference in their entirety.

To facilitate review of the various embodiments, the following explanations of terms are provided:

4-1BB: A co-stimulatory molecule expressed by T cell receptor (TCR)-activated lymphocytes, and by other cells including natural killer cells. Ligation of 4-1BB induces a signaling cascade that results in cytokine production, expression of anti-apoptotic molecules and an enhanced immune response.

Administration: To provide or give a subject an agent, such as a CAR or CAR-expressing cell provided herein, by any effective route. Exemplary routes of administration include, but are not limited to, oral, injection (such as subcutaneous, intramuscular, intradermal, intraperitoneal, intravenous, intraprostatic, and intratumoral), sublingual, rectal, transdermal, intranasal, vaginal and inhalation routes.

Antibody: A polypeptide ligand comprising at least one variable region that recognizes and binds (such as specifically recognizes and specifically binds) an epitope of an antigen, such as GPC1. Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles, and has some functionally similar to mammalian IgG and IgE. IgW and IgNAR antibodies are produced by cartilaginous fish, while IgX antibodies are found in amphibians.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., (JMB 273,927-948, 1997; the “Chothia” numbering scheme), and the ImMunoGeneTics (IMGT) database (see, Lefranc, Nucleic Acids Res 29:207-9, 2001; the “IMGT” numbering scheme). The Kabat and IMGT databases are maintained online.

A “single-domain antibody” refers to an antibody having a single domain (a variable domain) that is capable of specifically binding an antigen, or an epitope of an antigen, in the absence of an additional antibody domain. Single-domain antibodies include, for example, V_{H} domain antibodies, V_{NAR} antibodies, camelid V_{H}H antibodies, and V_{L} domain antibodies. V_{NAR} antibodies are produced by cartilaginous fish, such as nurse sharks, wobbegong sharks, spiny dogfish and bamboo sharks. Camelid V_{H}H antibodies are produced by several species including camel, llama, alpaca, dromedary, and guanaco, which produce heavy chain antibodies that are naturally devoid of light chains.

A “monoclonal antibody” is an antibody produced by a single clone of lymphocytes or by a cell into which the coding sequence of a single antibody has been transfected. Monoclonal antibodies are produced by methods known to those of skill in the art. Monoclonal antibodies include humanized monoclonal antibodies.

A “chimeric antibody” has framework residues from one species, such as human, and CDRs (which generally confer antigen binding) from another species.

A “humanized” antibody is an immunoglobulin including a human framework region and one or more CDRs from a non-human (for example a mouse, rabbit, rat, shark or synthetic) immunoglobulin. The non-human immunoglobulin providing the CDRs is termed a “donor,” and the human immunoglobulin providing the framework is termed an “acceptor.” In one embodiment, all CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions.

Binding affinity: Affinity of an antibody for an antigen. In one embodiment, affinity is calculated by a modification of the Scatchard method described by Frankel et al., Mol. Immunol., 16:101-106, 1979. In another embodiment, binding affinity is measured by an antigen/antibody dissociation rate. In another embodiment, binding affinity is measured by a competition radioimmunoassay. In another embodiment, binding affinity is measured by ELISA. In another embodiment, antibody affinity is measured by flow cytometry. In one embodiment, Kd is measured by a radiolabeled antigen binding assay (RIA) performed with the Fab version of an antibody of interest and its antigen (see, e.g., Chen et al., J. Mol. Biol. 293:865-881, 1999). In another example, Kd is measured using surface plasmon resonance assays using a BIACORES-2000 or a BIACORES-3000 (BIAcore, Inc., Piscataway, N.J.) at 25° C. with immobilized antigen CM5 chips at about 10 response units (RU).

An antibody that “specifically binds” an antigen (such as GPC1) is an antibody that binds the antigen with high affinity and does not significantly bind other unrelated antigens. In some examples, an antibody or fragment thereof (such as an anti-GPC1 antibody disclosed herein) specifically binds to a target (such as a GPC1) with a binding constant that is at least 10³ M⁻¹ greater, 10⁴ M⁻¹ greater or 10⁵ M⁻¹ greater than a binding constant for other molecules in a sample or subject. In some examples, an antibody (e.g., monoclonal antibody) or fragments thereof, has an equilibrium constant (Kd) of 10 nM or less, such as 9 nM or less, 8.1 nM or less, 8 nM or less, 7 nM or less, 6 nM or less, 6.5 nM or less, 6.3 nM or less, 5 nM or less, 4.3 nM or less, 4 nM or less, 3 nM or less, 2 nM or less, 1.5 nM or less, 1.5 nM or less, 1.4 nM or less, 1.3 nM or less, or 1.2 nM or less. For example, an antibody or fragment thereof binds to a target, such as GPC1 with a binding affinity of at least about 0.1 × 10⁻⁸ M, at least about 0.3 × 10⁻⁸ M, at least about 0.5 × 10⁻⁸ M, at least about 0.75 × 10⁻⁸ M, at least about 1.0 × 10⁻⁸ M, at least about 1.3 × 10⁻⁸ M at least about 1.5 × 10⁻⁸ M, or at least about 2.0 × 10⁻⁸ M, at least about 2.5 × 10⁻⁸, at least about 3.0 × 10⁻⁸, at least about 3.5 × 10⁻⁸, at least about 4.0 × 10⁻⁸, at least about 4.5 × 10⁻⁸, at least about 5.0 × 10⁻⁸ M, at least about 1 × 10⁻⁹ M, at least about 1.3 × 10⁻⁹ M, at least about 1.5 × 10⁻⁹ M, at least about 2 × 10⁻⁹ M, at least about 3 × 10⁻⁹ M, at least about 4 × 10⁻⁹ M, at least about 4.3 × 10⁻⁹ M, at least about 5 × 10⁻⁹ M, at least about 6 × 10⁻⁹ M, at least about 6.3 × 10⁻⁹ M, at least about 6.9 × 10⁻⁹ M, at least about 7 × 10⁻⁹ M, at least about 8 × 10⁻⁹ M, at least about 8.1 × 10⁻⁹ M, or at least about 10 × 10⁻⁹ M. In certain embodiments, a specific binding agent that binds to its target has a dissociation constant (Kd) of ≤100 nM, ≤10 nM, ≤9 nM, ≤8 nM, ≤7 nM, ≤6.9 nM, ≤6.5 nM, ≤6.3 nM, ≤5 nM, ≤4 nM, ≤4.5 nM, ≤3 nM, ≤2 nM, ≤1.5 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

Breast cancer: A type of cancer that forms in tissues of the breast, usually the ducts and lobules. Types of breast cancer include, for example, ductal carcinoma in situ, invasive ductal carcinoma, triple negative breast cancer, inflammatory breast cancer, metastatic breast cancer, medullary carcinoma, tubular carcinoma and mucinous carcinoma. Triple negative breast cancer refers to a type of breast cancer in which the cancer cells do not express estrogen receptors, progesterone receptors or significant levels of HER2/neu protein. Triple negative breast cancer is also called ER-negative PR-negative HER2/neu-negative breast cancer.

Chemotherapeutic agent: Any chemical agent with therapeutic usefulness in the treatment of diseases characterized by abnormal cell growth. Such diseases include tumors, neoplasms, and cancer. In one embodiment, a chemotherapeutic agent is an agent of use in treating a GPC1-positive tumor. In one embodiment, a chemotherapeutic agent is a radioactive compound. Exemplary chemotherapeutic agents that can be used with the methods provided herein are disclosed in Slapak and Kufe, Principles of Cancer Therapy, Chapter 86 in Harrison’s Principles of Internal Medicine, 14th edition; Perry et al., Chemotherapy, Ch. 17 in Abeloff, Clinical Oncology 2^{nd} ed., © 2000 Churchill Livingstone, Inc; Baltzer, L., Berkery, R. (eds.): Oncology Pocket Guide to Chemotherapy, 2nd ed. St. Louis, Mosby-Year Book, 1995; Fischer, D.S., Knobf, M.F., Durivage, H.J. (eds): The Cancer Chemotherapy Handbook, 4th ed. St. Louis, Mosby-Year Book, 1993). Combination chemotherapy is the administration of more than one agent to treat cancer. One example is the administration of GPC1-targeted CAR T cells used in combination with a radioactive or chemical compound. In one example, a chemotherapeutic agent is a biologic, such as a therapeutic antibody (e.g., therapeutic monoclonal antibody), such as an anti-GPC1 antibody, as well as other anti-cancer antibodies, such as anti-PD1 or anti-PDL1 (e.g., pembrolizumab and nivolumab), anti-CTLA4 (e.g., ipilimumab), anti-EGFR (e.g., cetuximab), anti-VEGF (e.g., bevacizumab), or combinations thereof (e.g., anti-PD-1 and anti-CTLA-4).

Chimeric antigen receptor (CAR): A chimeric molecule that includes an antigen-binding portion (such as a scFv or single-domain antibody) and a signaling domain, such as a signaling domain from a T cell receptor (for example, CD3ζ). Typically, CARs are comprised of an antigen-binding moiety, a transmembrane domain and an endodomain. The endodomain typically includes a signaling chain having an immunoreceptor tyrosine-based activation motif (ITAM), such as CD3ζ or FcεRIγ. In some instances, the endodomain further includes the intracellular portion of at least one additional co-stimulatory domain, such as CD28, 4-1BB (CD137), ICOS, OX40 (CD134), CD27 and/or DAP10. In some examples, the CAR is multispecific (such as bispecific) or bicistronic. A multispecific CAR is a single CAR molecule comprised of at least two antigen-binding domains (such as scFvs and/or single-domain antibodies) that each bind a different antigen or a different epitope on the same antigen (see, for example, US 2018/0230225). For example, a bispecific CAR refers to a single CAR molecule having two antigen-binding domains that each bind a different antigen. A bicistronic CAR refers to two complete CAR molecules, each containing an antigen-binding moiety that binds a different antigen. In some cases, a bicistronic CAR construct expresses two complete CAR molecules that are linked by a cleavage linker. Immune cells, such as T cells or NK cells, expressing a bispecific or bicistronic CAR can bind cells that express both of the antigens to which the binding moieties are directed (see, for example, Qin et al., Blood 130:810, 2017; and WO/2018/213337).

Colorectal cancer: A type of cancer that develops in the colon or the rectum. The most common type of colorectal cancer is colorectal adenocarcinoma, which accounts for approximately 95% of all colorectal cancers. Adenocarcinomas develop in the cells lining the inside of the colon and/or rectum. Other types of colorectal cancers include gastrointestinal carcinoid tumors, metastatic colorectal cancer, primary colorectal lymphoma (a type of non-Hodgkin’s lymphoma), gastrointestinal stromal tumors (classified as a sarcoma and arising from interstitial cells of Cajal), leiomyosarcoma (arising from smooth muscle cells) and colorectal melanoma.

Complementarity determining region (CDR): A region of hypervariable amino acid sequence that defines the binding affinity and specificity of an antibody. The light and heavy chains of a mammalian immunoglobulin each have three CDRs, designated L-CDR1, L-CDR2, L-CDR3 and H-CDR1, H-CDR2, H-CDR3, respectively. A single-domain antibody contains three CDRs, referred to herein as CDR1, CDR2 and CDR3.

Conservative variant: In the context of the present disclosure, “conservative” amino acid substitutions are those substitutions that do not substantially affect or decrease the affinity of a protein, such as an antibody, to GPC1. As one example, a monoclonal antibody that specifically binds GPC1 can include at most about 1, at most about 2, at most about 5, and most about 10, or at most about 15 conservative substitutions and specifically bind the GPC1 polypeptide. The term “conservative variant” also includes the use of a substituted amino acid in place of an unsubstituted parent amino acid, provided that the variant retains activity. Non-conservative substitutions are those that reduce an activity (such as affinity) of a protein.

Conservative amino acid substitution tables providing functionally similar amino acids are well known to one of ordinary skill in the art. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

In some embodiments herein, provided are amino acid sequences comprising no more than 10, no more than 9, no more than 8, no more than 7, no more than 6, no more than 5, no more than 4, no more than 3, no more than 2 or no more than 1 amino acid substitutions relative to any one of SEQ ID NOs: 2, 4, 6-23 and 30.

Contacting: Placement in direct physical association; includes both in solid and liquid form.

Degenerate variant: A polynucleotide encoding a polypeptide that includes a sequence that is degenerate as a result of the genetic code. There are 20 natural amino acids, most of which are specified by more than one codon. Therefore, all degenerate nucleotide sequences are included as long as the amino acid sequence of the polypeptide is unchanged.

Endometrial cancer: A type of cancer that forms in the endometrium, the tissue lining the uterus. Most endometrial cancers are adenocarcinomas, which arise from the epithelial cells of the endometrium.

Epitope: An antigenic determinant. These are particular chemical groups or peptide sequences on a molecule that are antigenic (that elicit a specific immune response). An antibody specifically binds a particular antigenic epitope on a polypeptide.

Framework region: Amino acid sequences interposed between CDRs. Framework regions include variable light and variable heavy framework regions. The framework regions serve to hold the CDRs in an appropriate orientation for antigen binding.

Fusion protein: A protein comprising at least a portion of two different (heterologous) proteins.

Glioma: A cancer of the brain and spinal cord that begins in glial cells, which are cells that surround and support nerve cells. Gliomas are classified based on the type of glial cells that produce the tumor. Types of gliomas include astrocytoma (including glioblastoma), ependymoma and oligodendroglioma, which originate in astrocytes, ependymal cells and oligodendrocytes, respectively.

Glypican-1 (GPC1): A member of the six-member glypican family of heparan sulfate proteoglycans (HSPGs) that are attached to the cell surface by a GPI anchor (Filmus et al., Genome Biol 9:224, 2008). GPC1 is overexpressed in certain types of cancer, such as pancreatic cancer (Kleeff et al., J Clin Invest 102:1662-1673, 1998), for example, pancreatic ductal adenocarcinoma (Frampton et al., Oncotarget 9:19006-19013, 2018; Kayed et al., Int J Oncol 29:1139-1148, 2006), glioma (Su et al., Am J Pathol 168:2014-2026, 2006), breast cancer (Matsuda et al., Cancer Res 61:5562-5569, 2001), ovarian cancer (Davies et al., Clin Cancer Res 10:5178-5186, 2004), and colorectal cancer (Li et al., Oncotarget 8:101189-101202, 2017). GPC1 genomic, mRNA and protein sequences are publicly available (see, for example, NCBI Gene ID 2817).

GPC1-positive cancer: A cancer that expresses or overexpresses GPC1. Examples of GPC1-positive cancers include, but are not limited to pancreatic cancer, colorectal cancer, liver cancer, glioma, lung cancer, head and neck cancer, thyroid cancer, osteosarcoma, endometrial cancer, breast cancer and ovarian cancer. In some embodiments, the GPC1-positive cancer has a low density of GPC1, such as no more than 2500, no more than 2000, or no more than 1500 molecules of GPC1 per cell, such as 1-2500, 100-2500, 1-2000, 100-1000, 1-1500, 100-1500, 1000-2500, 1000-2000, 500-2500, 500-2000, 500-1000, 1-100, 10-100, 10-1000, 10-2000, or 10-2500 molecules of GPC1 per cell.

Head and neck cancer: Cancer that forms in the squamous cells that line the mucosal surfaces inside the head and neck, such as inside the mouth, nose and throat. Head and neck cancer is often referred to as squamous cell carcinoma of the head and neck.

Heterologous: Originating from a separate genetic source or species.

Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4⁺ response or a CD8⁺ response. In another embodiment, the response is a B cell response, and results in the production of specific antibodies.

Isolated: An “isolated” biological component, such as a nucleic acid, protein (including antibodies) or organelle, has been substantially separated or purified away from other biological components in the environment (such as a cell) in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “labeled antibody” refers to incorporation of another molecule in the antibody. For example, the label is a detectable marker, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S, ¹¹C, ¹³N, ¹⁵O, ¹⁸F, ¹⁹F, ^{99m}Tc, ¹³¹I, ³ H, ¹⁴C, ^{15N,} ^{90y,} ⁹⁹Tc, ¹¹¹In and ¹²⁵I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

Linker: In some cases, a linker is a peptide within an antibody binding fragment (such as an Fv fragment) which serves to indirectly bond the variable heavy chain to the variable light chain. “Linker” can also refer to a peptide serving to link a targeting moiety, such as an antibody, to an effector molecule, such as a cytotoxin or a detectable label.

The terms “conjugating,” “joining,” “bonding” or “linking” refer to making two polypeptides into one contiguous polypeptide molecule, or to covalently attaching a radionuclide or other molecule to a polypeptide, such as an scFv. In the specific context, the terms include reference to joining a ligand, such as an antibody moiety, to an effector molecule. The linkage can be either by chemical or recombinant means. “Chemical means” refers to a reaction between the antibody moiety and the effector molecule such that there is a covalent bond formed between the two molecules to form one molecule.

Liver cancer: Any type of cancer occurring in liver tissue. The most common type of liver cancer is hepatocellular carcinoma (HCC), which develops in hepatocytes. Other types of liver cancer include cholangiocarcinoma, which develops in the bile ducts; liver angiosarcoma, which is a rare form of liver cancer that begins in the blood vessels of the liver; and hepatoblastoma, which is a very rare type of liver cancer found most often in children.

Lung cancer: Cancer that forms in tissues of the lung, usually in the cells lining air passages. The two main types are small cell lung cancer and non-small cell lung cancer (NSCLC). These types are diagnosed based on how the cells look under a microscope.

Mammal: This term includes both human and non-human mammals. Similarly, the term “subject” includes both human and veterinary subjects, such as mice, rats, cows, cats, dogs, pigs, and non-human primates.

Neoplasia, malignancy, cancer or tumor: A neoplasm is an abnormal growth of tissue or cells that results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.”

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Osteosarcoma: A type of cancer of the bone that generally affects the large bones of the arm or leg. Osteosarcoma is most common in young people and occurs more frequently in males than females. Osteosarcoma is also known as osteogenic sarcoma.

Ovarian cancer: Cancer that forms in tissues of the ovary. Most ovarian cancers are either ovarian epithelial carcinomas (cancer that begins in the cells on the surface of the ovary) or malignant germ cell tumors (cancer that begins in egg cells). Another type of ovarian cancer is stromal cell cancer, which originates in cells that release hormones and connect the different structures of the ovaries.

Pancreatic cancer: A disease in which malignant cells are found in the tissues of the pancreas. Pancreatic tumors can be either exocrine tumors or neuroendocrine tumors, based on the cell origin of the cancer. The vast majority (~94%) of pancreatic cancers are exocrine tumors. Exocrine cancers include, for example, adenocarcinoma (the most common type of exocrine tumor), acinar cell carcinoma, intraductal papillary-mucinous neoplasm (IPMN), and mucinous cystadenocarcinoma. In some examples, the pancreatic cancer is pancreatic ductal adenocarcinoma (PDAC). Pancreatic neuroendocrine tumors, also referred to as islet cell tumors, are classified by the type of hormones they produce. Exemplary neuroendocrine tumors include gastrinoma, glucaganoma, insulinoma, somatostatinoma, VIPoma (vasoactive intestinal peptide) and nonfunctional islet cell tumor.

Pharmaceutically acceptable carriers: The pharmaceutically acceptable carriers of use are conventional. Remington: The Science and Practice of Pharmacy, 22^{nd} ed., London, UK: Pharmaceutical Press, 2013,), describes compositions and formulations suitable for pharmaceutical delivery of the compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in tumor burden or a decrease in the number or size of metastases. “Ameliorating” refers to the reduction in the number or severity of signs or symptoms of a disease, such as cancer.

Purified: The term purified does not require absolute purity; rather, it is intended as a relative term. Thus, for example, a purified peptide preparation is one in which the peptide or protein is more enriched than the peptide or protein is in its natural environment within a cell. In one embodiment, a preparation is purified such that the protein or peptide represents at least 50% of the total peptide or protein content of the preparation. Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Recombinant: A recombinant nucleic acid is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques.

Sample (or biological sample): A biological specimen containing genomic DNA, RNA (including mRNA), protein, or combinations thereof, obtained from a subject. Examples include, but are not limited to, peripheral blood, tissue, cells, urine, saliva, tissue biopsy, fine needle aspirate, surgical specimen, and autopsy material. In one example, a sample includes a tumor biopsy, such as a tumor tissue biopsy.

Subject: Living multi-cellular vertebrate organisms, a category that includes both human and veterinary subjects, including human and non-human mammals. In one example, a subject has a GPC-1 positive cancer.

Synthetic: Produced by artificial means in a laboratory, for example a synthetic nucleic acid or protein (for example, an antibody) can be chemically synthesized in a laboratory.

Therapeutically effective amount: A quantity of a specific substance sufficient to achieve a desired effect in a subject being treated. For instance, this can be the amount necessary to inhibit or suppress growth of a tumor. In one embodiment, a therapeutically effective amount is the amount necessary to eliminate, reduce the size, or prevent metastasis of a tumor, such as reduce a tumor size and/or volume by at least 10%, at least 20%, at least 50%, at least 75%, at least 80%, at least 90%, at least 95%, or even 100%, and/or reduce the number and/or size/volume of metastases by at least 10%, at least 20%, at least 50%, at least 75%, at least 80%, at least 90%, at least 95%, or even 100%, for example as compared to a size/volume/number prior to treatment,. When administered to a subject, a dosage will generally be used that will achieve target tissue concentrations (for example, in tumors) that has been shown to achieve a desired in vitro effect.

Thyroid cancer: A type of cancer that forms in the tissues of the thyroid gland. Thyroid cancers are classified according to histopathological characteristic and include papillary thyroid cancer, follicular thyroid cancer, medullary thyroid cancer, poorly differentiated thyroid cancer, anaplastic thyroid cancer, thyroid lymphoma, squamous cell thyroid carcinoma and sarcoma of the thyroid.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art. In some examples, the vector is a viral vector, such as a lentiviral vector.

### III. Overview of Several Embodiments

The present disclosure describes GPC1-targeted chimeric antigen receptors (CARs) engineered to optimize the hinge and transmembrane regions for enhanced CAR T cell potency. It is disclosed herein that the hinge and transmembrane domain of GPC1-specific CARs exert a major effect on T cell function, particularly when GPC1 density on tumor cells is low. Evaluation of several different hinge sequences identified the 12-amino acid IgG4 hinge as optimal, most markedly for CARs containing an antigen-binding domain that targets a GPC1 epitope distal to the cell membrane. The optimized CARs include a transmembrane domain from either CD8α or CD28. Immune cells, such as T cells, natural killer cells or macrophages, expressing the optimized CARs can be used to treat solid tumors that express GPC1. In some examples, the disclosed CARs are comprised of an antibody (or antigen-binding fragment thereof) that has high affinity for GPC1 and binds a membrane distal epitope of GPC1 (e.g., the N-lobe of GPC1) or a membrane proximal epitope of GPC1 (e.g., the C-lobe of GPC1). GPC1-targeted CARs can be used to treat GPC1-expressing tumors, such as pancreatic cancer, colorectal cancer, liver cancer, glioma, lung cancer, head and neck cancer, thyroid cancer, osteosarcoma, endometrial cancer, breast cancer or ovarian cancer.

Provided herein are CARs that include an extracellular antigen-binding domain that specifically binds GPC1; an IgG4 hinge region; a transmembrane domain; an intracellular co-stimulatory domain; and an intracellular signaling domain. In some embodiments, the hinge region comprises or consists of the IgG4 hinge sequence set forth as SEQ ID NO: 7. In some embodiments, the transmembrane domain includes a CD28 transmembrane domain or a CD8α transmembrane domain.

In some embodiments, the antigen-binding domain of the CAR specifically binds GPC1 with high affinity. In some examples, the antigen-binding domain includes a GPC1-specific single-domain antibody or a GPC1-specific scFv. In some examples, the antigen-binding domain includes one or more CDR sequences (such as one, two or all three CDR sequences) from GPC1-specific single-domain antibody D4. In other examples, the antigen-binding domain includes one or more CDR sequences (such as one, two, three, four, five or all six CDR sequences) from GPC1-specific monoclonal antibody HM2.

In some embodiments, the antigen-binding domain of the CAR is a single-domain antibody that includes the CDR1, CDR2 and CDR3 sequences of SEQ ID NO: 6 (D4). In some examples, the CDR1, CDR2 and CDR3 sequences respectively comprise residues 31-35, 50-66 and 99-109 of SEQ ID NO: 6; residues 26-33, 51-58 and 97-108 of SEQ ID NO: 6; residues 27-33, 47-61 and 97-108 of SEQ ID NO: 6; or residues 26-35, 47-66 and 97-108 of SEQ ID NO: 6. In specific examples, the amino acid sequence of the single-domain antibody is at least 80%, at least 85%, at least 90%, at least 95%, at least 96%, at least 97%, at least 98%, or at least 99% identical to SEQ ID NO: 6 (and includes the CDR1, CDR2 and CDR3 sequences of SEQ ID NO: 6). In particular non-limiting examples, the amino acid sequence of the single-domain antibody comprises or consists of SEQ ID NO: 6.

In other embodiments, the antigen-binding domain of the CAR is a scFv that includes a variable heavy (VH) domain and a variable light (VL) domain and the VH domain includes the CDR1, CDR2 and CDR3 sequences of SEQ ID NO: 2 (HM2 VH domain), and the VL domain includes the CDR1, CDR2 and CDR3 sequences of SEQ ID NO: 4 (HM2 VL domain). In some examples, the VH domain CDR1, CDR2 and CDR3 sequences respectively comprise residues 31-35, 50-66 and 99-103 of SEQ ID NO: 2; residues 26-33, 51-58 and 97-103 of SEQ ID NO: 2; residues 27-35, 47-61 and 97-103 of SEQ ID NO: 2; or residues 26-35, 47-66 and 97-103 of SEQ ID NO: 2. In some examples, the VL domain CDR1, CDR2 and CDR3 sequences respectively comprise residues 24-39, 55-61 and 94-102 of SEQ ID NO: 4; residues 27-37, 55-57 and 94-101 of SEQ ID NO: 4; residues 28-39, 51-61 and 94-102 of SEQ ID NO: 4; or residues 24-39, 51-61 and 94-102 of SEQ ID NO: 4. In specific examples, the amino acid sequence of the VH domain is at least 80%, at least 85%, at least 90%, at least 95%, at least 96%, at least 97%, at least 98%, or at least 99% identical to SEQ ID NO: 2 (and includes the CDR1, CDR2 and CDR3 sequences of SEQ ID NO: 2); and/or the amino acid sequence of the VL domain is at least 80%, at least 85%, at least 90%, at least 95%, at least 96%, at least 97%, at least 98%, or at least 99% identical to SEQ ID NO: 4 (and includes the CDR1, CDR2 and CDR3 sequences of SEQ ID NO: 4). In particular non-limiting examples, the amino acid sequence of the VH domain comprises or consists of SEQ ID NO: 2; and/or the amino acid sequence of the VL domain comprises or consists of SEQ ID NO: 4. In other particular examples, the scFv includes the amino acid sequence of residues 25-265 of SEQ ID NO: 18 (the HM2 VH-linker-VL sequence).

In some embodiments, the transmembrane domain of the CAR includes a CD28 transmembrane domain, such as the CD28 transmembrane domain set forth herein as SEQ ID NO: 12. In other embodiments, the transmembrane domain of the CAR includes a CD8α transmembrane domain, such as the CD8α transmembrane domain set forth herein as SEQ ID NO: 11.

In some embodiments, the co-stimulatory domain of the CAR includes a 4-1BB signaling moiety, such as the 4-1BB signaling moiety set forth as SEQ ID NO: 13.

In some embodiments, the signaling domain of the CAR includes a CD3ζ signaling domain, such as the CD3ζ signaling domain set forth as SEQ ID NO: 14.

Also provided are isolated that cells that express a CAR disclosed herein. In some embodiments, the cell is an immune cell, such as a T cell, NK cell or macrophage.

Further provided are nucleic acid molecules that encode a disclosed CAR. In some embodiments, the nucleic acid molecule is operably linked to a promoter. In some embodiments, the nucleic acid molecule includes, in the 5′ to 3′ direction, a nucleic acid encoding a first granulocyte-macrophage colony stimulating factor receptor signal sequence (GMCSFRss); a nucleic acid encoding the antigen-binding domain; a nucleic acid encoding the IgG4 hinge region; a nucleic acid encoding the transmembrane domain; a nucleic acid encoding the co-stimulatory domain; a nucleic acid encoding the signaling domain; a nucleic acid encoding a self-cleaving 2A peptide; a nucleic acid encoding a second GMCSFRss; and a nucleic acid encoding a truncated human epidermal growth factor receptor (huEGFRt). In some examples, the nucleic acid molecule further includes a human elongation factor 1α (EF1α) promoter sequence 5′ of the nucleic acid encoding the first GMCSFRss (see WO 2019/094482, which is herein incorporated by reference in its entirety).

Vectors that include a nucleic acid molecule disclosed herein are further provided. In some examples, the vector is a viral vector, such as a lentiviral vector.

Also provided are isolated cells that include a nucleic acid molecule or vector disclosed herein. In some embodiments, the isolated cell is an immune cell, such as a T cell (such as a CTL), an NK cell or a macrophage.

Further provided are compositions that include a pharmaceutically acceptable carrier and a CAR, nucleic acid molecule, vector or cell disclosed herein.

Methods of treating a GPC1-positive cancer, or inhibiting tumor growth or metastasis of a GPC1-positive cancer, in a subject are also provided. In some embodiments, the methods include administering to the subject a therapeutically effective amount of a CAR, nucleic acid molecule, vector, cell or composition disclosed herein. In some examples, the GPC1-positive cancer is a solid tumor. In particular non-limiting examples, the GPC1-positive cancer is a pancreatic cancer, colorectal cancer, liver cancer, glioma, lung cancer, head and neck cancer, thyroid cancer, osteosarcoma, endometrial cancer, breast cancer or ovarian cancer. In some examples, the cancer has a low density of GPC1, such as no more than about 2500, no more than about 2000 or no more than about 1500 molecules of GPC1 per cell.

### IV. GPC1-Specific Antibody Sequences

The CARs disclosed herein include an antibody (or antigen-binding fragment thereof) that specifically binds GPC1. In some embodiments, the antibody is HM2, a mouse monoclonal antibody, or D4, a single-domain camel antibody. The nucleotide and amino acid sequences of HM2 and D4 are provided below. Tables 1A, 1B and 2 list the amino acid positions of the CDR1, CDR2 and CDR3 of each antibody, as determined using either Kabat, IMGT, or Paratome, or a combination of all three. One of skill in the art could readily determine the CDR boundaries using an alternative numbering scheme, such as the Chothia numbering scheme.

HM2 VH DNA (SEQ ID NO: 1)

GAGGTTCAGCTGCAGCAGTCTGGGGCTGAGCTTGTGAGGCCAGGGGCCTC AGTCAAGTTGTCCTGCACAGCTTCTGGCTTTAACATTAAAGACGACTATA
TGCACTGGGTGAAGCAGAGGCCTGAACAGGGCCTGGAGTGGATTGGATGG
ATTGATCCTGAGAATGGTGATACTGAATATGCCTCGAAGTTCCAGGGCAA
GGCCACTATAACAGCAGACACATCCTCCAACACAGCCTACCTGCAGCTCA
GCAGCCTGACATCTGAGGACACTGCCGTCTATTACTGTACTCGTAGCTCC
GTAGGCTACTGGGGCCAAGGCACCACTCTCACAGTCTCCTCA

HM2 VH Protein (SEQ ID NO: 2)

EVQLQQSGAELVRPGASVKLSCTASGFNIKDDY MHWVKQRPEQGLEWIGW IDPENGDT EYASKFQGKATITADTSSNTAYLQLSSLTSEDTAVYYCTRSS VGY WGQGTTLTVSS

(Underline = Kabat CDRs; Bold = IMGT CDRs; Italics = Paratome CDRs)

HM2 VL DNA (SEQ ID NO: 3)

GATGTTGTGATGACCCAAACTCCACTCTCCCTGCCTGTCAGTCTTGGAGA
TCAAGCCTCCATCTCTTGCAGATCTAGTCAGAGCCTTGTACACAGTAATG
GAAACACCTATTTACATTGGTACCTGCAGAAGCCAGGCCAGTCTCCAAAG
CTCCTGATCTACAAAGTTTCCAACCGATTTTCTGGGGTCCCAGACAGGTT
CAGTGGCAGTGGATCAGGGACTTATTTCACACTCAAGATCAGCAGAGTGG
AGGCTGAGGATCTGGGAGTTTATTTCTGCTCTCAAAGAACACATGTTCCG
TACACGTTCGGAGGGGGGACCAAGCTGGAGATAAAA

HM2 VL Protein (SEQ ID NO: 4)

DVVMTQTPLSLPVSLGDQASISCRSSQSLVHSNGNTY LH WYLQKPGQSPK
LLIYKVS NRFS GVPDRFSGSGSGTYFTLKISRVEAEDLGVYFCSQRTHVP YT FGGGTKLEIK

(Underline = Kabat CDRs; Bold = IMGT CDRs; Italics = Paratome CDRs)

TABLE 1A

Location of CDRs in HM2 VH domain amino acid sequence (SEQ ID NO: 2)

| Numbering Scheme | CDR1 | CDR2 | CDR3 |
|---|---|---|---|
| Kabat | 31-35 | 50-66 | 99-103 |
| IMGT | 26-33 | 51-58 | 97-103 |
| Paratome | 27-35 | 47-61 | 97-103 |
| Combined | 26-35 | 47-66 | 97-103 |

TABLE 1B

Location of CDRs in HM2 VL domain amino acid sequence (SEQ ID NO: 4)

| Numbering Scheme | CDR1 | CDR2 | CDR3 |
|---|---|---|---|
| Kabat | 24-39 | 55-61 | 94-102 |
| IMGT | 27-37 | 55-57 | 94-101 |
| Paratome | 28-39 | 51-61 | 94-102 |
| Combined | 24-39 | 51-61 | 94-102 |

D4 DNA (SEQ ID NO: 5)

CAGGTGCAGCTGGTGGAGTCTGGGGGAGGCTTGGTGCAGCCCGGGGGGTC
TCTGAGACTCTCCTGTGTAGCCTCTGGATACAGCTACAGTATTGGTTACA
TGGCCTGGTTCCGCCAGGCCCCAGGAAAGGAGCGCGCGTGGGTCGCGTCT
CGATATACTGGTGACGGTGGCGCAGTCTTTGACGACGCCGTGAAGGGCCG
ATTCACCACCTCCCAAGAGAGTGCCGGGAACACGTTCGATTTGCAAATGG
ACAGCCTGAAACCTGAGGACACTGCCATGTACTATTGCGCAGCGAAAGGG
CCCGGTTTCGGGCGGTGGGAGTACTGGGGCCGGGGGACCCAGGTCACCGT
CTCCTCA

D4 Protein (SEQ ID NO: 6)

QVQLVESGGGLVQPGGSLRLSCVASGYSYSIGY MA WFRQAPGKERAWVAS RYTGDGGAVFDDAVKGRFTTSQESAGNTFDLQMDSLKPEDTAMYYCAAKG PGFGRWEY WGRGTQVTVSS

(Underline = Kabat CDRs; Bold = IMGT CDRs; Italics = Paratome CDRs)

TABLE 2

Location of CDRs in the D4 amino acid sequence (SEQ ID NO: 6)

| Numbering Scheme | CDR1 | CDR2 | CDR3 |
|---|---|---|---|
| Kabat | 31-35 | 50-66 | 99-109 |
| IMGT | 26-33 | 51-58 | 97-108 |
| Paratome | 27-33 | 47-61 | 97-108 |
| Combined | 26-35 | 47-66 | 97-108 |

### V. GPC1-Targeted CAR Sequences

The HM2 scFv and D4 single-domain antibody were used to generate several different CAR constructs utilizing different hinge regions and transmembrane (TM) domains. In the CAR amino acid sequences provided below, the antigen-binding sequence (HM2 VH-linker-VL; or D4 single-domain) is underlined, the hinge region (CD8α, IgG4, IgG4-CH3 or IgG4-CH2-CH3) is in bold and the TM domain (CD8α or CD28) is in italics.

HM2-CD8 hinge-CD8 TM CAR (SEQ ID NO: 18)

MLLLVTSLLLCELPHPAFLLIPHMEVQLQQSGAELVRPGASVKLSCTASG FNIKDDYMHWVKQRPEQGLEWIGWIDPENGDTEYASKFQGKATITADTSS NTAYLQLSSLTSEDTAVYYCTRSSVGYWGQGTTLTVSSGGGGSGGGGSGG GGSDVVMTQTPLSLPVSLGDQASISCRSSQSLVHSNGNTYLHWYLQKPGQ SPKLLIYKVSNRFSGVPDRFSGSGSGTYFTLKISRVEAEDLGVYFCSQRT HVPYTFGGGTKLEIKTSTTTPAPRPPTPAPTIASQPLSLRPEACRPAAGG AVHTRGLDFACDIYIWAPLAGTCGVLLLSLVITKRGRKKLLYIFKQPFMR
PVQTTQEEDGCSCRFPEEEEGGCELRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPREGRGSLLTCGDVE
ENPGPMLLLVTSLLLCELPHPAFLLIPRKVCNGIGIGEFKDSLSINATNI
KHFKNCTSISGDLHILPVAFRGDSFTHTPPLDPQELDILKTVKEITGFLL
IQAWPENRTDLHAFENLEIIRGRTKQHGQFSLAVVSLNITSLGLRSLKEI
SDGDVIISGNKNLCYANTINWKKLFGTSGQKTKIISNRGENSCKATGQVC
HALCSPEGCWGPEPRDCVSCRNVSRGRECVDKCNLLEGEPREFVENSECI
QCHPECLPQAMNITCTGRGPDNCIQCAHYIDGPHCVKTCPAGVMGENNTL
VWKYADAGHVCHLCHPNCTYGCTGPGLEGCPTNGPKIPSIATGMVGALLL
LLVVALGIGLFM

TABLE 3

Features of the HM2-CD8 hinge-CD8 TM CAR

| Feature | Residues of SEQ ID NO: 18 |
|---|---|
| GMCSFRss | 1-22 |
| Restriction site | 23-24 |
| HM2 (VH-linker-VL) | 25-265 |
| Restriction site | 266-267 |
| CD8α hinge | 268-312 |
| CD8α transmembrane domain | 313-333 |
| 4-1BB co-stimulatory domain | 334-375 |
| CD3ζ signaling domain | 376-487 |
| T2A | 488-505 |
| GMCSFRss | 506-527 |
| hEGFRt | 528-862 |

D4-CD8 hinge-CD8 TM CAR (SEQ ID NO: 19)

MLLLVTSLLLCELPHPAFLLIPHMQVQLVESGGGLVQPGGSLRLSCVASG YSYSIGYMAWFRQAPGKERAWVASRYTGDGGAVFDDAVKGRFTTSQESAG NTFDLQMDSLKPEDTAMYYCAAKGPGFGRWEYWGRGTQVTVSSTSTTTPA PRPPTPAPTIASQPLSLRPEACRPAAGGAVHTRGLDFACDIYIWAPLAGT CGVLLLSLVITKRGRKKLLYIFKQPFMRPVQTTQEEDGCSCRFPEEEEGG
CELRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTAT
KDTYDALHMQALPPREGRGSLLTCGDVEENPGPMLLLVTSLLLCELPHPA
FLLIPRKVCNGIGIGEFKDSLSINATNIKHFKNCTSISGDLHILPVAFRG
DSFTHTPPLDPQELDILKTVKEITGFLLIQAWPENRTDLHAFENLEIIRG
RTKQHGQFSLAVVSLNITSLGLRSLKEISDGDVIISGNKNLCYANTINWK
KLFGTSGQKTKIISNRGENSCKATGQVCHALCSPEGCWGPEPRDCVSCRN
VSRGRECVDKCNLLEGEPREFVENSECIQCHPECLPQAMNITCTGRGPDN
CIQCAHYIDGPHCVKTCPAGVMGENNTLVWKYADAGHVCHLCHPNCTYGC
TGPGLEGCPTNGPKIPSIATGMVGALLLLLVVALGIGLFM

TABLE 4

Features of the D4-CD8 hinge-CD8 TM CAR

| Feature | Residues of SEQ ID NO: 19 |
|---|---|
| GMCSFRss | 1-22 |
| Restriction site | 23-24 |
| D4 antibody | 25-143 |
| Restriction site | 144-145 |
| CD8α hinge | 146-190 |
| CD8α transmembrane domain | 191-211 |
| 4-1BB co-stimulatory domain | 212-253 |
| CD3ζ signaling domain | 254-365 |
| T2A | 366-383 |
| GMCSFRss | 384-405 |
| hEGFRt | 406-740 |

D4-IgG4 hinge-CD8 TM CAR (SEQ ID NO: 20)

MLLLVTSLLLCELPHPAFLLIPHMQVQLVESGGGLVQPGGSLRLSCVASG YSYSIGYMAWFRQAPGKERAWVASRYTGDGGAVFDDAVKGRFTTSQESAG NTFDLQMDSLKPEDTAMYYCAAKGPGFGRWEYWGRGTQVTVSSTSESKYG PPCPPCPIYIWAPLAGTCGVLLLSLVITKRGRKKLLYIFKQPFMRPVQTT
QEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGER
RRGKGHDGLYQGLSTATKDTYDALHMQALPPREGRGSLLTCGDVEENPGP
MLLLVTSLLLCELPHPAFLLIPRKVCNGIGIGEFKDSLSINATNIKHFKN
CTSISGDLHILPVAFRGDSFTHTPPLDPQELDILKTVKEITGFLLIQAWP
ENRTDLHAFENLEIIRGRTKQHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFM

TABLE 5

Features of the D4-IgG4 hinge-CD8 TM CAR

| Feature | Residues of SEQ ID NO: 20 |
|---|---|
| GMCSFRss | 1-22 |
| Restriction site | 23-24 |
| D4 antibody | 25-143 |
| Restriction site | 144-145 |
| IgG4 hinge | 146-157 |
| CD8α transmembrane domain | 158-178 |
| 4-1BB co-stimulatory domain | 179-220 |
| CD3ζ signaling domain | 221-332 |
| T2A | 333-350 |
| GMCSFRss | 351-372 |
| hEGFRt | 373-707 |

D4-IgG4 hinge-CD28 TM CAR (SEQ ID NO: 21)

MLLLVTSLLLCELPHPAFLLIPHMOVOLVESGGGLVOPGGSLRLSCVASG YSYSIGYMAWFRQAPGKERAWVASRYTGDGGAVFDDAVKGRFTTSQESAG NTFDLQMDSLKPEDTAMYYCAAKGPGFGRWEYWGRGTQVTVSSTSESKYG PPCPPCPFWVLVVVGGVLACYSLLVTVAFIIFWVKRGRKKLLYIFKQPFM
RPVQTTQEEDGCSCRFPEEEEGGCELRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPREGRGSLLTCGDV
EENPGPMLLLVTSLLLCELPHPAFLLIPRKVCNGIGIGEFKDSLSINATN
IKHFKNCTSISGDLHILPVAFRGDSFTHTPPLDPQELDILKTVKEITGFL
LIQAWPENRTDLHAFENLEIIRGRTKQHGQFSLAVVSLNITSLGLRSLKE
ISDGDVIISGNKNLCYANTINWKKLFGTSGQKTKIISNRGENSCKATGQV
CHALCSPEGCWGPEPRDCVSCRNVSRGRECVDKCNLLEGEPREFVENSEC
IQCHPECLPQAMNITCTGRGPDNCIQCAHYIDGPHCVKTCPAGVMGENNT
LVWKYADAGHVCHLCHPNCTYGCTGPGLEGCPTNGPKIPSIATGMVGALL
LLLVVALGIGLFM

TABLE 6

Features of the D4-IgG4 hinge-CD28 TM CAR

| Feature | Residues of SEQ ID NO: 21 |
|---|---|
| GMCSFRss | 1-22 |
| Restriction site | 23-24 |
| D4 antibody | 25-143 |
| Restriction site | 144-145 |
| IgG4 hinge | 146-157 |
| CD28 transmembrane domain | 158-184 |
| 4-1BB co-stimulatory domain | 185-226 |
| CD3ζ signaling domain | 227-338 |
| T2A | 339-356 |
| GMCSFRss | 357-378 |
| hEGFRt | 379-713 |

D4-IgG4 hinge-CH3-CD28 TM CAR (SEQ ID NO: 22)

MLLLVTSLLLCELPHPAFLLIPHMQVQLVESGGGLVQPGGSLRLSCVASG YSYSIGYMAWFRQAPGKERAWVASRYTGDGGAVFDDAVKGRFTTSQESAG NTFDLQMDSLKPEDTAMYYCAAKGPGFGRWEYWGRGTQVTVSSTSESKYG PPCPPCPGQPREPQVYTLPPSQEEMTKNQVSLTCLVKGFYPSDIAVEWES NGQPENNYKTTPPVLDSDGSFFLYSRLTVDKSRWQEGNVFSCSVMHEALH NHYTQKSLSLSLGKFWVLVVVGGVLACYSLLVTVAFIIFWVKRGRKKLLY
IFKQPFMRPVQTTQEEDGCSCRFPEEEEGGCELRVKFSRSADAPAYQQGQ
NQLYNELNLGRREEYDVLDKRRGRDPEMGGKPRRKNPQEGLYNELQKDKM
AEAYSEIGMKGERRRGKGHDGLYQGLSTATKDTYDALHMQALPPREGRGS
LLTCGDVEENPGPMLLLVTSLLLCELPHPAFLLIPRKVCNGIGIGEFKDS
LSINATNIKHFKNCTSISGDLHILPVAFRGDSFTHTPPLDPQELDILKTV
KEITGFLLIQAWPENRTDLHAFENLEIIRGRTKQHGQFSLAVVSLNITSL
GLRSLKEISDGDVIISGNKNLCYANTINWKKLFGTSGQKTKIISNRGENS
CKATGQVCHALCSPEGCWGPEPRDCVSCRNVSRGRECVDKCNLLEGEPRE
FVENSECIQCHPECLPQAMNITCTGRGPDNCIQCAHYIDGPHCVKTCPAG
VMGENNTLVWKYADAGHVCHLCHPNCTYGCTGPGLEGCPTNGPKIPSIAT
GMVGALLLLLVVALGIGLFM

TABLE 7

Features of the D4-IgG4 hinge-CH3-CD28 TM CAR

| Feature | Residues of SEQ ID NO: 22 |
|---|---|
| GMCSFRss | 1-22 |
| Restriction site | 23-24 |
| D4 antibody | 25-143 |
| Restriction site | 144-145 |
| IgG4 hinge | 146-157 |
| CH3 domain | 158-264 |
| CD28 transmembrane domain | 265-291 |
| 4-1BB co-stimulatory domain | 292-333 |
| CD3ζ signaling domain | 334-445 |
| T2A | 446-463 |
| GMCSFRss | 464-485 |
| hEGFRt | 486-820 |

D4-IgG4 hinge-CH2CH3-CD28 TM CAR (SEQ ID NO: 23)

MLLLVTSLLLCELPHPAFLLIPHMQVQLVESGGGLVQPGGSLRLSCVASG YSYSIGYMAWFRQAPGKERAWVASRYTGDGGAVFDDAVKGRFTTSQESAG NTFDLQMDSLKPEDTAMYYCAAKGPGFGRWEYWGRGTQVTVSSTSESKYG PPCPPCPAPPVAGPSVFLFPPKPKDTLMISRTPEVTCVVVDVSQEDPEVQ FNWYVDGVEVHNAKTKPREEQFQSTYRVVSVLTVLHQDWLNGKEYKCKVS NKGLPSSIEKTISKAKGQPREPQVYTLPPSQEEMTKNQVSLTCLVKGFYP SDIAVEWESNGQPENNYKTTPPVLDSDGSFFLYSRLTVDKSRWQEGNVFS CSVMHEALHNHYTQKSLSLSLGKFWVLVVVGGVLACYSLLVTVAFIIFWV
KRGRKKLLYIFKQPFMRPVQTTQEEDGCSCRFPEEEEGGCELRVKFSRSA
DAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQA
LPPREGRGSLLTCGDVEENPGPMLLLVTSLLLCELPHPAFLLIPRKVCNG
IGIGEFKDSLSINATNIKHFKNCTSISGDLHILPVAFRGDSFTHTPPLDP
QELDILKTVKEITGFLLIQAWPENRTDLHAFENLEIIRGRTKQHGQFSLA
VVSLNITSLGLRSLKEISDGDVIISGNKNLCYANTINWKKLFGTSGQKTK
IISNRGENSCKATGQVCHALCSPEGCWGPEPRDCVSCRNVSRGRECVDKC
NLLEGEPREFVENSECIQCHPECLPQAMNITCTGRGPDNCIQCAHYIDGP
HCVKTCPAGVMGENNTLVWKYADAGHVCHLCHPNCTYGCTGPGLEGCPTN
GPKIPSIATGMVGALLLLLVVALGIGLFM

TABLE 8

Features of the D4-IgG4 hinge-CH2CH3-CD28 TM CAR

| Feature | Residues of SEQ ID NO: 23 |
|---|---|
| GMCSFRss | 1-22 |
| Restriction site | 23-24 |
| D4 antibody | 25-143 |
| Restriction site | 144-145 |
| IgG4 hinge | 146-157 |
| CH2 domain | 158-266 |
| CH3 domain | 267-373 |
| CD28 transmembrane domain | 374-400 |
| 4-1BB co-stimulatory domain | 401-442 |
| CD3ζ signaling domain | 443-554 |
| T2A | 555-572 |
| GMCSFRss | 573-594 |
| hEGFRt | 595-929 |

### VI. Chimeric Antigen Receptors (CARs)

Disclosed herein are GPC1-specific CARs (also known as chimeric T cell receptors, artificial T cell receptors or chimeric immunoreceptors) and cells (for example, T cells, NK cells or macrophages) engineered to express CARs. Generally, CARs include a binding moiety, an extracellular hinge/spacer element, a transmembrane region and an intracellular domain that performs signaling functions (Cartellieri et al., J Biomed Biotechnol 2010:956304, 2010; Dai et al., J Natl Cancer Inst 108(7):djv439, 2016). In many instances, the binding moiety is an antigen binding fragment of a monoclonal antibody, such as a scFv or single-domain antibody. The spacer/hinge region typically includes sequences from IgG subclasses, such as IgG1, IgG4, IgD and CD8 domains. The transmembrane domain can be can derived from a variety of different T cell proteins, such as CD3ζ, CD4, CD8 or CD28.

While an entire intracellular T cell signaling domain can be employed in a CAR, in many cases it is not necessary to use the entire chain. To the extent that a truncated portion of the intracellular T cell signaling domain is used, such truncated portion may be used in place of the intact chain as long as it transduces the relevant T cell effector function signal. Examples of intracellular T cell signaling domains for use in the disclosed CARs include the cytoplasmic sequences of the T cell receptor (TCR) and co-stimulatory molecules that act in concert to initiate signal transduction following antigen receptor engagement, as well as any derivatives or variants of these sequences and any synthetic sequence that has the same functional capability. Several different intracellular domains have been used to generate CARs. For example, the intracellular domain can consist of a signaling chain having an ITAM, such as CD3ζ or FcεRIγ. In some instances, the intracellular domain further includes the intracellular portion of at least one additional co-stimulatory domain. The co-stimulatory domain refers to a portion of the CAR comprising the intracellular domain of a costimulatory molecule. A costimulatory molecule is a cell surface molecule other than an antigen receptor or their ligands that is required for an efficient response of lymphocytes to an antigen. Co-stimulatory molecules include, for example, CD28, 4-1BB (CD137, TNFRSF9), OX-40 (CD134), ICOS, CD27 and/or DAP10.

The CAR can also include a signal peptide sequence, e.g., N-terminal to the antigen binding domain. The signal peptide sequence can be any suitable signal peptide sequence, such as a signal sequence from granulocyte-macrophage colony-stimulating factor receptor (GMCSFR), immunoglobulin light chain kappa, or IL-2. While the signal peptide sequence may facilitate expression of the CAR on the surface of the cell, the presence of the signal peptide sequence in an expressed CAR is not necessary in order for the CAR to function. Upon expression of the CAR on the cell surface, the signal peptide sequence may be cleaved off of the CAR. Accordingly, in some embodiments, the CAR lacks a signal peptide sequence.

In some embodiments, the CARs disclosed herein are expressed from a construct (such as from a lentivirus vector) that also expresses a truncated version of human EGFR (huEGFRt; discussed in more detail in section VII below). The CAR and huEGFRt are separated by a self-cleaving peptide sequence (such as T2A) such that upon expression in a transduced cell, the CAR is cleaved from huEGFRt (see WO 2019/094482, which is herein incorporated by reference in its entirety).

In some embodiments disclosed herein, the CAR constructs encode the following features, in the N-terminal to C-terminal direction: a first GMCSFRss (for example, SEQ ID NO: 16); an antigen-binding domain (for example, the HM2 scFv or D4 single-domain antibody); a hinge (such as the IgG4 hinge of SEQ ID NO: 7); a transmembrane domain (such as the CD8α or CD28 transmembrane domain); a co-stimulatory domain (such as 4-1BB); a signaling domain (such as CD3ζ); a self-cleaving peptide sequence (such as T2A); a second GMCSFRss (for example, SEQ ID NO: 16); and huEGFRt (for example, SEQ ID NO: 17).

Immune cells, such as T cells, NK cells or macrophages, expressing the CARs disclosed herein can be used to target a specific cell type, such as a tumor cell, for example a GPC1-positive tumor cell. The use of immune cells (such as T cells) expressing CARs is more universal than standard CTL-based immunotherapy because immune cells expressing CARs are HLA unrestricted and can therefore be used for any patient having a tumor that expresses the target antigen.

Accordingly, provided herein are CARs that include a GPC1-specific antibody (or binding fragment thereof). Also provided are isolated nucleic acid molecules and vectors encoding the CARs, and host cells, such as T cells, NK cells or macrophages, expressing the CARs. Immune cells expressing CARs comprised of a GPC1-specific monoclonal antibody can be used for the treatment of cancers that express GPC1, such as pancreatic cancer, colorectal cancer, liver cancer, glioma, lung cancer, head and neck cancer, thyroid cancer, osteosarcoma, endometrial cancer, breast cancer or ovarian cancer.

### VII. Truncated Human EGFR (huEGFRt)

The human epidermal growth factor receptor is comprised of four extracellular domains, a transmembrane domain and three intracellular domains. The EGFR domains are found in the following N-terminal to C-terminal order: Domain I - Domain II - Domain III - Domain IV -transmembrane (TM) domain - juxtamembrane domain - tyrosine kinase domain - C-terminal tail. Domain I and Domain III are leucine-rich domains that participate in ligand binding. Domain II and Domain IV are cysteine-rich domains and do not make contact with EGFR ligands. Domain II mediates formation of homo- or hetero-dimers with analogous domains from other EGFR family members, and Domain IV can form disulfide bonds with Domain II. The EGFR TM domain makes a single pass through the cell membrane and may play a role in protein dimerization. The intracellular domain includes the juxtamembrane domain, tyrosine kinase domain and C-terminal tail, which mediate EGFR signal transduction (Wee and Wang, Cancers 9(52), doi:10.3390/cancers9050052; Ferguson, Annu Rev Biophys 37:353-373, 2008; Wang et al., Blood 118(5):1255-1263, 2011).

A truncated version of human EGFR, referred to herein as “huEGFRt” includes only Domain III, Domain IV and the TM domain. Thus, huEGFRt lacks Domain I, Domain II, and all three intracellular domains. huEGFRt is not capable of binding EGF and lacks signaling activity. However, this molecule retains the capacity to bind particular EGFR-specific monoclonal antibodies, such as FDA-approved cetuximab (PCT Publication No. WO 2011/056894, which is herein incorporated by reference).

Transduction of immune cells, such as T cells, NK cells or macrophages, with a construct (such as a lentivirus vector) encoding both huEGFRt and a tumor antigen-specific CAR disclosed herein allows for selection of transduced T cells using labelled EGFR monoclonal antibody cetuximab (ERBITUX™). For example, cetuximab can be labeled with biotin and transduced immune cells can be selected using anti-biotin magnetic beads, which are commercially available (such as from Miltenyi Biotec). Co-expression of huEGFRt also allows for in vivo tracking of adoptively transferred CAR-expressing cells. Furthermore, binding of cetuximab to immune cells expressing huEGFRt induces cytotoxicity of ADCC effector cells, thereby providing a mechanism to eliminate transduced immune cells in vivo (Wang et al., Blood 118(5):1255-1263, 2011), such as at the conclusion of therapy.

In some embodiments herein, the amino acid sequence of huEGFRt is at least 80%, at least 85%, at least 90%, at least 95%, at least 96%, at least 97%, at least 98% or at least 99% identical to SEQ ID NO: 17. In some examples, the amino acid sequence of huEGFRt comprises or consists of SEQ ID NO: 17. In other embodiments, the amino acid sequence of huEGFRt comprises no more than 10, no more than 9, no more than 8, no more than 7, no more than 6, no more than 5, no more than 4, no more than 3, no more than 2 or no more than 1 amino acid substitutions relative to SEQ ID NO: 17. In some examples, the amino acid substitutions are conservative substitutions.

### VIII. CAR-Expressing Cell Compositions

Compositions are provided that include CAR-expressing cells in combination with one or more pharmaceutically or physiologically acceptable carriers, diluents or excipients. The CAR-expressing cells can be T cells, such as CD3⁺ T cells, such as CD4⁺ and/or CD8⁺ T cells, NK cells, macrophages or any other suitable immune cell. Such compositions may include buffers such as neutral buffered saline, phosphate buffered saline and the like; carbohydrates such as glucose, mannose, sucrose, dextrans, or mannitol; proteins; polypeptides or amino acids such as glycine; antioxidants; chelating agents such as EDTA or glutathione; adjuvants (e.g., aluminum hydroxide); and preservatives. The cells can be autologous to the recipient. However, the cells can also be heterologous (allogeneic).

With regard to the cells, a variety of aqueous carriers can be used, for example, buffered saline and the like, for introducing the cells. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration in these formulations can vary widely, and will be selected primarily based on fluid volumes, viscosities, body weight and the like in accordance with the particular mode of administration selected and the subject’s needs.

The precise amount of the composition to be administered can be determined by a physician with consideration of individual differences in age, weight, tumor size, extent of metastasis, and condition of the patient (subject). It can generally be stated that a pharmaceutical composition comprising the CAR-expressing immune cells (T cells, macrophages and/or NK cells) described herein may be administered at a dosage of 10⁴ to 10⁹ cells/kg body weight, such as 10⁵ to 10⁶ cells/kg body weight, including all integer values within those ranges. Exemplary doses are 10⁶ cells/kg to about 10⁸ cells/kg, such as from about 5 × 10⁶ cells/kg to about 7.5 × 10⁷ cells/kg, such as at about 2.5 × 10⁷ cells/kg, or at about 5.0 × 10⁷ cells/kg.

A composition can be administered once or multiple times, such as 2, 3, 4, 5, 6, 7, 8, 9 or 10 times at these dosages. The composition can be administered by using infusion techniques known in immunotherapy (see, e.g., Rosenberg et al., New Eng. J. of Med. 319:1676, 1988). The compositions can be administered daily, weekly, bimonthly or monthly. In some non-limiting examples, the composition is formulated for intravenous administration and is administered multiple times. The quantity and frequency of administration will be determined by such factors as the condition of the subject, and the type and severity of the subject’s disease, although appropriate dosages may be determined by clinical trials.

In some embodiments, the CAR-encoding nucleic acid molecule is introduced into cells, such as T cells, NK cells or macrophages, and the subject receives an initial administration of cells, and one or more subsequent administrations of the cells, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR-expressing cells are administered to the subject (e.g., human) per week, e.g., 2, 3, or 4 administrations of the CAR-expressing cells of the disclosure are administered per week. In one embodiment, the subject receives more than one administration of the CAR-expressing cells per week (e.g., 2, 3 or 4 administrations per week) (also referred to as a cycle), followed by a week of no CAR-expressing cell administrations, and then one or more additional administration of the CAR-expressing cells (e.g., more than one administration of the CAR-expressing cells per week) is administered to the subject. In another embodiment, the subject (e.g., a human subject) receives more than one cycle of CAR-expressing cells, and the time between each cycle is less than 10, 9, 8, 7, 6, 5, 4, or 3 days. In one embodiment, the CAR-expressing cells are administered every other day for 3 administrations per week. In another embodiment, the CAR-expressing cells are administered for at least two, three, four, five, six, seven, eight or more weeks. The dosage of the above treatments to be administered to a patient will vary with the precise nature of the condition being treated and the recipient of the treatment. The scaling of dosages for human administration can be performed according to art-accepted practices.

In some embodiments, CAR-expressing cells are able to replicate in vivo resulting in long-term persistence that can lead to sustained tumor control. In various aspects, the T or NK cells administered to the subject, or the progeny of these cells, persist in the subject for at least four months, five months, six months, seven months, eight months, nine months, ten months, eleven months, twelve months, thirteen months, fourteen months, fifteen months, sixteen months, seventeen months, eighteen months, nineteen months, twenty months, twenty-one months, twenty-two months, twenty-three months, or for years after administration of the cells to the subject. In other embodiments, the cells and their progeny are present for less than six months, five months, four months, three months, two months, or one month, e.g., three weeks, two weeks, one week, after administration of the CAR-expressing T cells to the subject.

The administration of the compositions may be carried out in any convenient manner, including by injection, ingestion, transfusion, implantation or transplantation. The disclosed compositions can be administered to a patient trans-arterially, subcutaneously, intradermally, intratumorally, intranodally, intramedullary, intramuscularly, by intravenous (i.v.) injection, intraprostatically (e.g., for a prostate cancer), or intraperitoneally. In some embodiments, the compositions are administered to a patient by intradermal or subcutaneous injection. In other embodiments, the compositions of the present invention are administered by i.v. injection. The compositions can also be injected directly into a tumor or lymph node.

In some embodiments, subjects can undergo leukapheresis, wherein leukocytes are collected, enriched, or depleted ex vivo to select and/or isolate the cells of interest, e.g., T cells, macrophages and/or NK cells. These cell isolates may be expanded by methods known in the art and treated such that one or more CAR constructs can be introduced, thereby creating an autologous cell that expresses the CAR. In some embodiments herein, CAR-expressing cells are generated using lentiviral vectors expressing the CAR and a truncated form of the human EGFR (huEGFRt). Co-expression of huEGFRt allows for selection and purification of CAR-expressing immune cells using an antibody that recognizes huEGFRt (e.g., cetuximab, see PCT Publication No. WO 2011/056894, which is herein incorporated by reference), which is described above in section VII.

In some embodiments, immune cells (such as T cells, NK cells and/or macrophages) are isolated from peripheral blood by lysing the red blood cells and in some instances depleting the monocytes, for example, by centrifugation through a PERCOLL™ gradient or by counterflow centrifugal elutriation. A specific subpopulation of T cells, such as CD3+, C28+, CD4+, CD8+, CD45RA4+, and CD45RO+ T cells, can be further isolated by positive or negative selection techniques. For example, T cells can isolated by incubation with anti-CD3/anti-CD28 (e.g., 3x28)-conjugated beads, such as DYNABEADS® M-450 CD3/CD28 T, for a time period sufficient for positive selection of the desired T cells, see U.S. Published Application No. US20140271635 A1. In a non-limiting example, the time period is about 30 minutes. In other non-limiting examples, the time period ranges from 30 minutes to 36 hours or longer and all integer values there between. In further non-limiting examples, the time period is at least 1, 2, 3, 4, 5, or 6 hours, 10 to 24 hours, 24 hours or longer. Longer incubation times can be used to isolate T cells in any situation where there are few T cells as compared to other cell types, such in isolation from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. Multiple rounds of selection can also be used.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for cells by negative selection, a monoclonal antibody cocktail typically includes antibodies to CD14, CD20, CD11b, CD16, HLA-DR, and CD8. A T cell population can be selected that expresses one or more cytokines. Methods for screening for cell expression are disclosed in PCT Publication No. WO 2013/126712.

For isolation of a desired population of cells by positive or negative selection, the concentration of cells and surface (e.g., particles such as beads) can be varied to ensure maximum contact of cells and beads. In some embodiments, a concentration of 1 billion cells/ml is used. In further embodiments, greater than 100 million cells/ml is used. In other embodiments, a concentration of cells of 10, 15, 20, 25, 30, 35, 40, 45, 50, 55, 60, 65, 70, 75, 80, 85, 90, 95, or 100 million cells/ml is used. Without being bound by theory, using high concentrations can result in increased cell yield, cell activation, and cell expansion. Lower concentrations of cells can also be used. Without being bound by theory, significantly diluting the mixture of T cells and surface (e.g., particles such as beads), interactions between the particles and cells is minimized. This selects for cells that express high amounts of desired antigens to be bound to the particles. For example, T cells express higher levels of CD28 and are more efficiently captured than CD8+ T cells in dilute concentrations. In some embodiments, the concentration of cells used is 5×10⁶/ml. In other embodiments, the concentration used can be from about 1×10⁵/ml to 1×10⁶/ml, and any integer value in between.

### IX. Methods of Treatment

Provided herein are methods of treating cancer in a subject by administering to the subject a therapeutically effective amount of a GPC1-targeted CAR immune cell (such as T cell, NK cell or macrophage) disclosed herein. Also provided herein is a method of inhibiting tumor growth or metastasis in a subject by administering to the subject a therapeutically effective amount of a GPC1-targeted CAR immune cell disclosed herein. Thus, in some examples, the methods decrease the size, volume and/or weight of a tumor by at least 10%, at least 20%, at least 30%, at least 50%, at least 50%, at least 75%, at least 90%, at least 95%, at least 98%, at least 99% or 100%, for example relative to the size, volume and/or weight of the tumor prior to treatment. In some examples, the methods decrease the size, volume and/or weight of a metastasis by at least 10%, at least 20%, at least 30%, at least 50%, at least 50%, at least 75%, at least 90%, at least 95%, at least 98%, at least 99% or 100%, for example relative to the size, volume and/or weight of the metastasis prior to treatment. In some examples, the methods increase the survival time of a subject with a GPC1-positive cancer by at least 3 months, at least 6 months, at least 9 months, at least 12 months, at least 18 months, at least 24 months, at last 36 months, at least 48 months, or at least 60 months, for example relative to the survival time in an absence of the treatment provided herein. In some examples, combinations of these effects are achieved.

Specifically provided is a method of treating a GPC1-positive cancer in a subject. In some embodiments, the method includes administering to the subject a therapeutically effective amount of an isolated immune cell that comprises a nucleic acid molecule encoding a GPC1-targeted CAR and a huEGFRt, or administering a therapeutically effective amount of an isolated immune cell co-expressing a GPC1-targeted CAR and a huEGFRt. In some embodiments, the GPC1-positive cancer is a pancreatic cancer, colorectal cancer, liver cancer, glioma, lung cancer, head and neck cancer, thyroid cancer, osteosarcoma, endometrial cancer, breast cancer or ovarian cancer. In some examples the GPC1-positive cancer is one having a low density of GPC1, such as a cancer expressing less than about 2500, less than about 2000 or less than about 1500 molecules of GPC1 per cell. In some instances, the GPC1-positive cancer having a low-density of GPC1 is a pancreatic cancer.

In some embodiments of the methods disclosed herein, the isolated immune cells are T lymphocytes. In some examples, the T lymphocytes are autologous T lymphocytes. In other embodiments, the isolated host cells are NK cells or macrophages.

A therapeutically effective amount of a CAR-expressing immune cell will depend upon the severity of the disease, the type of disease, and the general state of the patient’s health. A therapeutically effective amount of CAR-expressing immune cells and compositions thereof is that which provides either subjective relief of a symptom(s) or an objectively identifiable improvement as noted by the clinician or other qualified observer (such as a decrease in tumor volume or metastasis).

Administration of the CAR-expressing cells and compositions disclosed herein can also be accompanied by administration of other anti-cancer agents or therapeutic treatments (such as surgical resection of a tumor). Any suitable anti-cancer agent can be administered in combination with the compositions disclosed herein. Exemplary anti-cancer agents include, but are not limited to, chemotherapeutic agents, such as, for example, mitotic inhibitors, alkylating agents, antimetabolites, intercalating antibiotics, growth factor inhibitors, cell cycle inhibitors, enzymes, topoisomerase inhibitors, anti-survival agents, biological response modifiers, anti-hormones (e.g., anti-androgens) and anti-angiogenesis agents. Other anti-cancer treatments include radiation therapy and antibodies (e.g., mAbs) that specifically target cancer cells or other cells (e.g., anti-PD-1, anti-CLTA4, anti-EGFR, or anti-VEGF). In one example, a cancer is treated by administering a GPC1-targeted CAR immune cell (such as T cell, NK cell or macrophage) disclosed herein and one or more therapeutic mAbs, such as one or more of a PD-L1 antibody (e.g., durvalumab, KN035, cosibelimab, BMS-936559, BMS935559, MEDI-4736, MPDL-3280A, or MEDI-4737), or CLTA-4 antibody (e.g., ipilimumab or tremelimumab). In one example, a cancer is treated by administering a GPC1-targeted CAR immune cell (such as T cell, NK cell or macrophage) disclosed herein and one or more mAbs, for example: 3F8, Abagovomab, Adecatumumab, Afutuzumab, Alacizumab, Alemtuzumab, Altumomab pentetate, Anatumomab mafenatox, Apolizumab, Arcitumomab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuximab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin, Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab, Morolimumab, Nacolomab tafenatox, Naptumomab estafenatox, Necitumumab, Nimotuzumab, Nofetumomab merpentan, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Satumomab pendetide, Sibrotuzumab, Sonepcizumab, Tacatuzumab tetraxetan, Taplitumomab paptox, Tenatumomab, TGN1412, Ticilimumab (tremelimumab), Tigatuzumab, TNX-650, Trastuzumab, Tremelimumab, Tucotuzumab celmoleukin, Veltuzumab, Volociximab, Votumumab, Zalutumumab, or combinations thereof.

Non-limiting examples of alkylating agents include nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine).

Non-limiting examples of antimetabolites include folic acid analogs (such as methotrexate), pyrimidine analogs (such as 5-FU or cytarabine), and purine analogs, such as mercaptopurine or thioguanine.

Non-limiting examples of natural products include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitomycin C), and enzymes (such as L-asparaginase).

Non-limiting examples of miscellaneous agents include platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide).

Non-limiting examples of hormones and antagonists include adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testerone proprionate and fluoxymesterone). Exemplary chemotherapy drugs that can be used in combination with the methods provided herein include Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, while some more newer drugs include Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol.

Non-limiting examples of immunomodulators that can be used include AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, and TNF (tumor necrosis factor; Genentech).

Another treatment that can be used in combination with those provided herein is surgical treatment, for example surgical resection of the cancer or a portion of it. Another example of a treatment is radiotherapy, for example administration of radioactive material or energy (such as external beam therapy) to the tumor site to help eradicate the tumor or shrink it prior to surgical resection.

The following examples are provided to illustrate certain particular features and/or embodiments. These examples should not be construed to limit the disclosure to the particular features or embodiments described.

### EXAMPLES

### Example 1: Materials and Methods

This example describes the materials and experimental procedures used for the studies described in Example 2.

### Cell Culture

The A431 (epidermal carcinoma) and HEK-293T cell lines were from American Type Culture Collection (ATCC). H8 is a transfected A431 cell line stably expressing human GPC1. The aforementioned cell lines were cultured in DMEM supplemented with 10% FBS, 1% L-glutamine, and 1% penicillin-streptomycin at 37° C. in a humidified atmosphere with 5% CO₂. PBMCs were isolated from the blood of healthy donors by Ficoll (GE Healthcare) according to the manufacturer’s instructions. These cells were grown in RPMI-1640 medium supplemented with 10% FBS, 1% L-glutamine, and 1% penicillin-streptomycin at 37° C. in a humidified atmosphere with 5% CO₂. The hTERT-HPNE cell line was from ATCC and cultured according to the provider’s instructions. A431, H8, 2B9 and T3M4 cell lines were engineered to express luciferase (Luc) and GFP.

### Isolation of anti-GPC1 Antibodies

The isolation of mouse mAb against glypican-1 was described previously (Phung et al., MAbs 2012;4:592-599). Briefly, the process includes peptide synthesis, immunization of mice, spleen cell fusion, hybridoma selection and expansion. The C-terminal peptide consisting of 50 residues was synthesized (GenScript). Hybridoma cells were screening via ELISA and flow cytometry. The HM2 clone, which demonstrated the highest affinity and greatest specific binding, was chosen for purification. The D4 antibody was isolated from a large phage-displayed camel single-domain antibody library constructed using the EASeL method described previously (Feng et al., Antib Ther 2019;2:1-11). Through three sequential rounds of panning on an ELISA plate (Thermo Fisher Scientific) coated with human GPC1 in phosphate-buffered saline (PBS), GPC1-specific phages were enriched. Single colonies were then picked and identified by performing phage ELISA.

### Elisa

Mouse hybridoma supernatant containing 1 µg/ml of each mAb was incubated with plates coated with human GPC1 through GPC6 purchased from R&D Systems. Binding was detected with a goat anti-mouse IgG conjugated with horseradish peroxidase (HRP) (Jackson ImmunoResearch). The D4 camel single domain antibody at 1 µg/ml was incubated with human GPC1 through GPC6 and mouse GPC1 proteins. Binding was detected with an anti-FLAG HRP-conjugated antibody (Sigma-Aldrich). For the sandwich ELISA, a plate was coated with HM2 mAb in PBS. Recombinant human GPC1-hFc protein at concentrations of 5 µg/ml and 1 µg/ml were then added to the plate. After three washes, D4 was added to the plate at concentrations of 0.4 µg/ml and 2 µg/ml. The bound D4 was detected by adding the anti-FLAG HRP-conjugated antibody.

### Flow Cytometry

Cytokines and chemokines were also analyzed using the LEGENDplex Human Essential Immune Response Panel (Biolegend) as per the manufacturer’s instructions. Analysis was performed by flow cytometry using a LSR-Fortessa cytometer (Beckman Coulter) and data was processed using LEGENDplex Data Analysis Software (Biolegend).

T3M4 pancreatic tumor cells were incubated with mouse hybridoma supernatant containing 10 µg/ml of each mAb. Cell binding was then detected with a goat anti-mouse IgG conjugated with phycoerythrin (PE). For analysis of GPC1 expression on the cell surface, tumor cells were incubated with 10 µg/ml of HM2 or D4, and detected with a goat anti-mouse IgG conjugated with allophycocyanin (APC) or an anti-FLAG antibody conjugated with APC, respectively. To measure lentiviral transduction efficiencies, CAR expression on T cells was detected with the anti-EGFR human monoclonal antibody cetuximab (Erbitux) and goat-anti-human IgG conjugated with PE. All secondary antibodies unless otherwise noted were purchased from Jackson ImmunoResearch. Data acquisition was performed using FACSCantoII (BD Biosciences) and analyzed using FloJo software (Tree Star).

### Antibody Binding Assay

The binding kinetics of HM2 and D4 antibodies was measured with the Octet RED96 system (FortéBio). For HM2, His-tagged GPC1 protein was immobilized onto a Ni-NTA biosensor, which was subsequently used in association and dissociation measurements for a time window of 600 s and 1800 s, respectively. For D4, His-tagged D4 antibody was used to load the Ni-NTA biosensor, and serial diluted antigen human GPC1-hFc protein was used for the binding assay. Data analysis was performed using the FortéBio analysis software.

### Immunohistochemistry

A pancreatic tumor tissue microarray was purchased from US Biomax. The sections were stained with 1 µg/ml HM2 mAb. The immunohistochemical staining was performed by Histoserv Inc.

### Negative Stain EM Preparation and Data Collection

The HM2 antigen-binding fragment (Fab) was prepared using a Fab preparation kit (Thermo Fisher Scientific). GPC1 protein was mixed with HM2 Fab at 1:1 molar ratio in PBS. In addition, GPC1 protein was mixed with D4-LR immunotoxin that lacks domain II of Pseudomonas exotoxin (PE) at 1:1 molar ratio in PBS. A 3 µL aliquot containing ~0.01 mg/mL of the sample was applied for 20 seconds onto a carbon-coated 200 Cu mesh grid (Electron Microscopy Sciences, Protochips, Inc.) that had been glow discharged at 30 mA for 30 seconds (Pelco easiGlow, Ted Pella, Inc.), then negatively stained with 0.7% (w/v) uranyl formate for 40 seconds. Data was collected using a Tecnai FEI T20 electron microscope operating at 200 kV, with an electron dose of ~40 e-/Å2 and a magnification of 100,000 x that resulted in a pixel size of 2.19 Å at the specimen plane. Images were acquired with an Eagle 2kx2k CCD camera (FEI) using a nominal defocus of 1100 nm and the SerialEM software (Mastronarde, J Struct Biol 2005;152:36-51).

### Negative Stain EM Data Processing and Model Building

Particles were selected from the micrographs, extracted, and reference-free 2D class averages were obtained using RELION 3.0.8 (Fernandez-Leiro and Scheres, Acta Crystallogr D Struct Biol 2017;73:496-502). After 2D sorting, particles were subject to 3D classification, requesting 6 classes, and starting with an initial model of the GPC2 unliganded and filtered to 60 Å resolution without imposing symmetry. The best class for the complex was selected for further refinement without imposing symmetry in RELION 3.0.8. After examination, one of the initial 3D models, which reasonably represents a protein complex, was chosen as the template for particle picking from the raw images. A new set of particles were picked using a rather high threshold (>0.9). This procedure is used to avoid picking too many particles that are not protein complexes but rather just components of the complex. 2D classifications were performed on the new set of particles, first with 50 classes, then with 20 classes. Bad particles were discarded after each 2D classification. 3D classifications were followed with either 5 or 3 classes. Bad particles were again discarded after this step. Lastly, the particles which were contributing to the best 3D classification model were selected for 3D refinement. Final model was produced when the 3D refinements were converged. All the above procedures were carried out in RELION-3.0.8.

### Reverse Transcriptase Polymerase Chain Reaction (RT-PCR)

mRNA was isolated using a QuickPrep mRNA Purification kit (GE Healthcare), and first strand cDNAs were synthesized using a SuperScript III First-Strand Synthesis System (Thermo Fisher Scientific) according to the manufacturers’ instructions. Primers designed to amplify GPC1 and β-actin are listed below.

| Name | Sequence (5′ to 3′) | SEQ ID NO: |
|---|---|---|
| Human GPC1-forward | GCAGCGTGCACACGTGGCTG | 24 |
| Human GPC1-reverse | CTGGCCCTTACAGTAGCCAGGC | 25 |
| Human β-actin-forward | CACCATTGGCAATGAGCGGTTC | 26 |
| Human β-actin-reverse | AGGTCTTTGCGGATGTCCACGT | 27 |

### Immunoblotting

Cell lysates were loaded onto a 4-20% SDS-PAGE gel for electrophoresis. HM2 was used to detect GPC1 expression. The anti-GAPDH antibody was obtained from Cell Signaling Technology.

### Generation of GPC1-specific CAR T Cells

The HM2 variable regions were cloned using 5′RACE with modified primers and conducted as described previously (Sivasubramanian et al., Proteins 2009;74:497-514; Zhang and Ho, Sci Rep 2016;6:33878). The antigen recognition regions from the HM2 (pMH304) or D4 (pMH305) antibody was subcloned into a lentiviral vector containing expressing cassettes encoding the hinge and TM regions of CD8, a 4-1BB costimulatory domain, intracellular CD3ζ, the self-cleaving T2A sequence, and the truncated human epidermal growth factor receptor (hEGFRt) for cell tracking and ablation. A CD19-targeted CAR with the hinge and TM from CD8α was used as a control. The CD8 hinge in the initial D4 CAR construct was replaced with a modified human IgG4 hinge (S→P substitution) (Hudecek et al., Clin Cancer Res 2013;19:3153-3164) followed by either CD8 TM (pMH382) or CD28 TM (pMH377) domain.

An additional spacer domain derived from a modified human IgG4-Fc was added into the D4-IgG4 hinge-CD28 TM CAR construct: D4-IgG4 hinge-CH₃-CD28 TM CAR (pMH378) and D4-IgG4 hinge-CH₂CH₃-CD28 TM CAR (pMH379) (Hudecek et al., Cancer Immunol Res 2015;3:125-135). Specifically, the first six amino acids (APEFLG; SEQ ID NO: 87) were replaced with five amino acids (APPVA; residues 158-162 of SEQ ID NO: 23), and a mutation (N297Q) at a glycosylation site in the CH₂ domain. Additionally, two cysteine residues in the hinge of D4-IgG4 hinge-CD28 TM CAR were mutated to serine (pMH383). The CAR T cells were produced as described previously (Li et al., Gastroenterology 2020;158:2250-2265; Li et al., Proc Natl Acad Sci U S A 2017;114:E6623-E6631).

### CRISPR/Cas9-mediated Editing of GPC1

The lentiCRISPRv2 expression vector was obtained from Addgene (plasmid #52961). Two single-guide RNAs (sgRNAs) targeting the promoter region of GPC1 were cloned into the lentiCRISPRv2 vector following the protocol as described previously (Sanjana et al., Nat Methods 11:783-784, 2014). The sgRNAs sequences are listed below. GPC1 knockout (KO)-T3M4 cells were obtained by single-clone selection.

| sgRNA | Sequence (5′-3′) | SEQ ID NO: |
|---|---|---|
| sgRNA 1 | CCTCTCCCGCGGCCGCCTAG | 28 |
| sgRNA 2 | GAGCGAGCGTTCGGACCTCG | 29 |

### In Vitro Functional Assays

The cytolytic activity of GPC1-targeted CAR T cells was determined using a luciferase-based assay as previously described (Li et al., Gastroenterology 2020;158:2250-2265; Li et al., Proc Natl Acad Sci U S A 2017;114:E6623-E6631). In brief, GPC1-targeted CAR T cells and mock T cells were co-cultured with GPC1-positive pancreatic cancer cells (T3M4), GPC1-overexpressing cells (2B9 derived from KLM1, H8 derived from A431), and GPC1-negative cells (GPC1 knockout-T3M4, A431) at different ratios for 24 hours. All tumor cells were engineered to express luciferase (Luc) and GFP. The luciferase activity was measured using the luciferase assay system (Promega) on Victor (PerkinElmer). IFN-γ, TNF-α and IL-2 secretion in the co-cultured supernatants were measured by ELISA (R&D Systems).

### Animal Studies

Five-week-old female NOD/SCID/IL-2Rgc^{null} (NSG) mice (NCI CCR Animal Resource Program/NCI Biological Testing Branch) were housed and treated under the protocol approved by the Institutional Animal Care and Use Committee at the NIH. For the intraperitoneal (i.p.) 2B9 model, 2 million Luc-expressing 2B9 (2B9-Luc) tumor cells were injected i.p. into mice. Mice with established tumors were then randomly allocated into 3 groups and injected i.p. once with 10 million T cells as follows: (a) un-transduced T cells (Mock); (b) HM2 CAR T cells; and (c) D4 CAR T cells. For the i.p. T3M4 model, 2 million T3M4-Luc tumor cells were injected i.p. into mice. Mice with established tumors were randomly allocated into groups including mock and various formats of D4 CAR T cells with different hinge and TM domains. Mock or D4 CAR T cells were infused i.p. once at a dose of 5 or 10 million cells. To detect the tumor growth and survival of mice, all mice were injected i.p. weekly with 3 mg D-luciferin (PerkinElmer) and imaged 10 minutes later using Xenogen IVIS Lumina (PerkinElmer). Living Image software was used to analyze the bioluminescence signal flux for each mouse as photons per second per square centimeter per steradian (photons/s/cm²/sr).

### Droplet Digital PCR (ddPCR)

Tissues were homogenized using the Bullet Blender, and genomic DNA from cells was isolated using the FlexiGene DNA kit (QIAGEN). ddPCR experiments were performed on a QX200 ddPCR system (Bio-Rad) according to the manufacturer’s instructions. The primer and probe sequences were previously described (Li et al., Gastroenterology 2020;158:2250-2265).

### Integration Site Analysis

CAR lentivector integration site analysis was performed using linker mediated PCR as described previously (De Ravin et al., Sci Transl Med 2016;8:335ra57; Maldarelli et al., Science 2014;345:179-183). Briefly, sample DNA was randomly sheared, end-repaired, and ligated to a linker. The integration site was amplified with one primer specific to the lentivector LTR and another primer specific to the linker. The amplified product was subjected to high-throughput Illumina Sequencing. Integration sites in the sample were identified and quantified for further analysis. The primer sequences were previously described (Li et al., Gastroenterology 2020;158:2250-2265).

### Example 2: CAR T Cells That Target a Membrane Distal or Membrane-Proximal Site of GPC1 and Have an IgG4 Hinge

This example describes the finding that GPC1-targeted CAR T cells having a relatively short hinge region from IgG4 exhibit improved reactivity against low GPC1-expressing tumor cells compared to CAR T cells with a longer hinge region.

### Isolation of High Affinity GPC1-Specific Antibodies

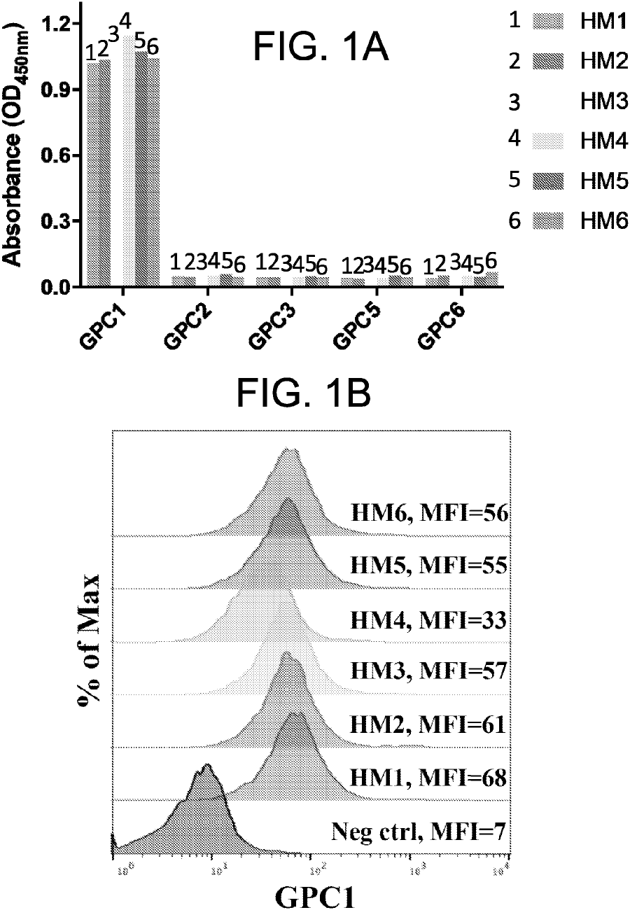

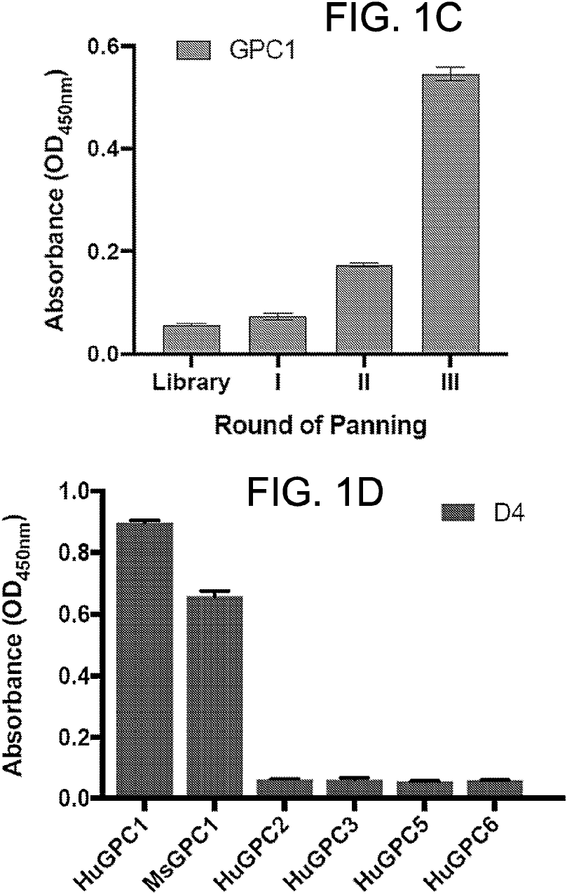

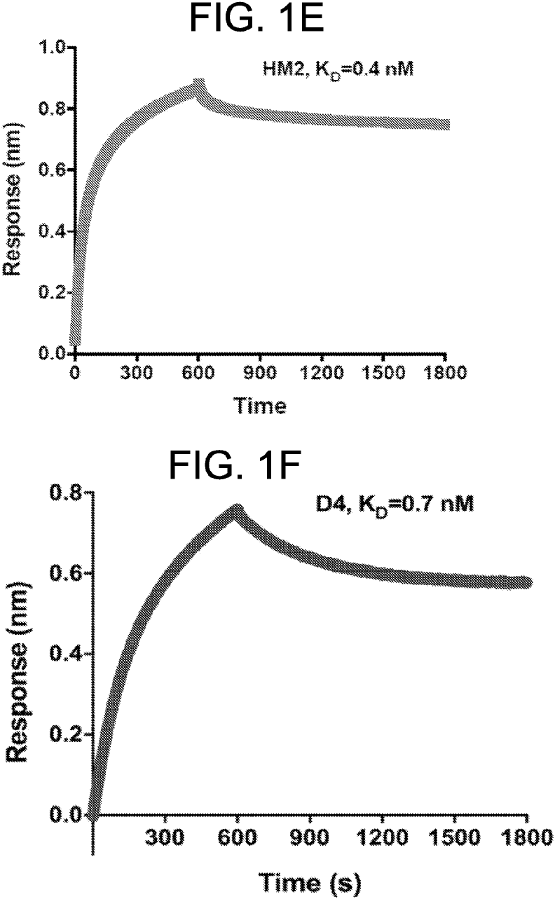

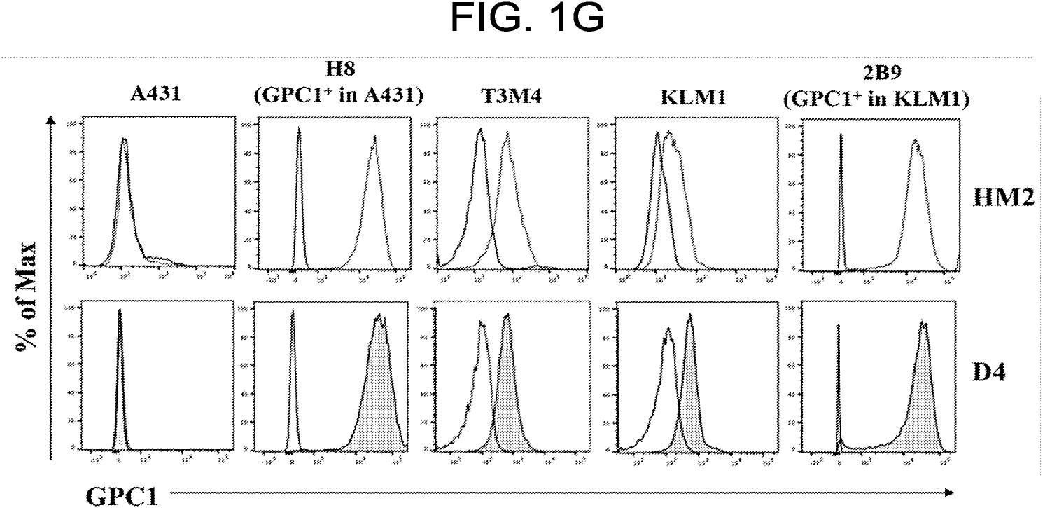

Although glypican members share ~25% amino acid similarity, their C-lobe regions close to the cell membrane are low in sequence similarity (Iozzo RV, Proteoglycans : structure, biology, and molecular interactions. New York: Marcel Dekker, 2000). Previous studies also demonstrate that CARs targeting membrane-proximal epitopes have superior antitumor activity compared with those incorporating other binding domains (Li et al., Gastroenterology 2020;158:2250-2265; Haso et al., Blood 2013;121:1165-1174). To isolate mouse mAbs having membrane-proximal GPC1-specific epitopes, mice were immunized with the C-lobe region of GPC1. As shown in FIG. 1A, six mAbs (HM1 through HM6) were recovered from three parental clones, which all specifically reacted to human GPC1. Although they bound to GPC1 expressed on the T3M4 pancreatic cancer cell line with similar affinity (FIG. 1B), the HM2 clone was chosen for the following studies as it showed the highest protein production yield among all the mAbs (Table 9).

TABLE 9

Protein production yield of anti-GPC1 mouse monoclonal antibodies

| Antibody | HM1 | HM2 | HM3 | HM4 | HM5 | HM6 |
|---|---|---|---|---|---|---|
| Concentration (µg/ml) | 12.3 | 22.3 | 7.4 | 7.6 | 4.1 | 7.8 |

To identify a single domain antibody specific for GPC1, a phage-displayed camel single domain antibody library was screened. As shown in FIG. 1C, phage pools after three rounds of panning exhibited enhanced binding to GPC1. The D4 clone was identified by monoclonal ELISA and sequencing (FIG. 1D). D4 specifically recognized human GPC1, but not other human glypican members. It also cross-reacted with mouse GPC1. The kinetic analysis using Octet revealed that both HM2 and D4 bound to human GPC1 stably with high affinity (FIGS. 1E and 1F). The K_{D} value of HM2 and D4 for GPC1 protein was 0.4 nM and 0.7 nM, respectively. The binding of HM2 and D4 to GPC1 on live cells was also examined by flow cytometry. Both antibodies bound equally well to GPC1-expressing T3M4 and KLM1 pancreatic cancer cells, GPC1-overexpressing A431 cells (H8) and GPC1-overexpressing KLM1 cells (2B9) (FIG. 1G). Conversely, these antibodies did not bind GPC1-negative A431 cells or GPC1-knockout (KO) T3M4 cells, indicating that binding is antigen-specific. Taken together, a mouse mAb (HM2) and a camel single domain antibody (D4) that specifically bind to GPC1 protein were successfully identified.

### HM2 and D4 Bind to Different Epitopes on GPC1

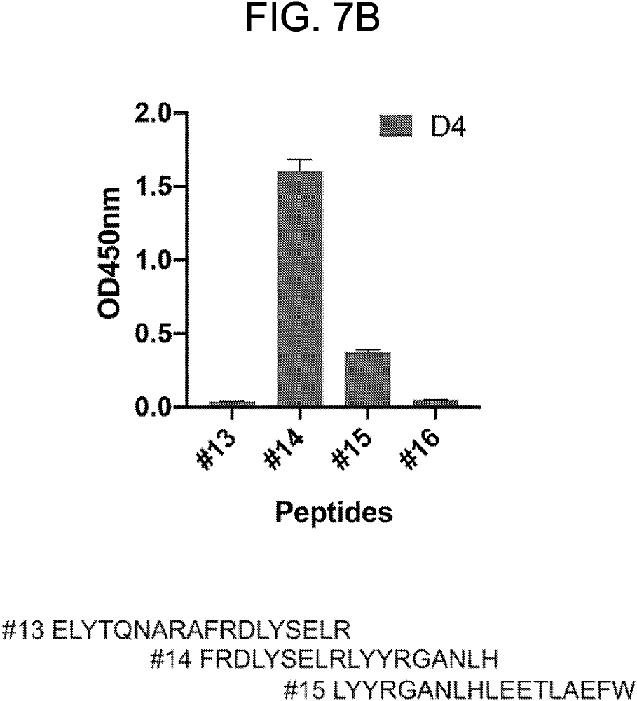

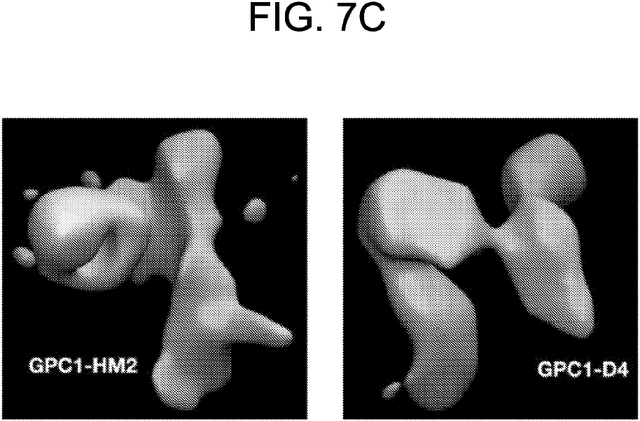

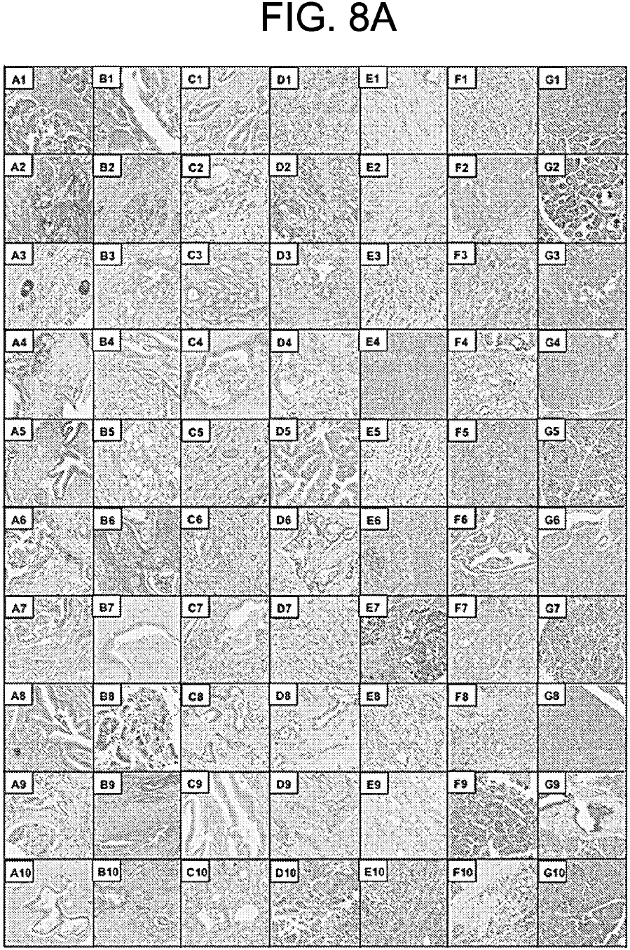

To identify the epitopes of HM2 and D4, a GPC1 peptide library that comprises 18 amino acid peptides with a 9-amino acid overlap with adjacent peptides was generated. The sequences are listed in the Table 10. As shown in FIGS. 7A and 7B, HM2 specifically reacted with peptide 53 (SEQ ID NO: 83), while D4 recognized epitopes comprising both peptide 14 (SEQ ID NO: 44) and peptide 15 (SEQ ID NO: 45). To further elucidate the binding epitopes of HM2 and D4 on GPC1, negative stain electron microscopy (EM) was applied to analyze the structure of the GPC1:HM2 antibody-binding fragment (Fab) complex and GPC1:D4-LR complex. FIG. 7C shows enlarged views of a 2D class average of GPC1 in complex with HM2 Fab and GPC1 in complex with D4-LR. D4-LR is an immunotoxin that lacks domain II of PE.

TABLE 10

Amino acid sequences of GPC1 peptides

| Peptide name | Sequence | SEQ ID NO: |
|---|---|---|
| peptide 1 | DPASKSRSCGEVRQIYGA | 31 |
| peptide 2 | GEVRQIYGAKGFSLSDVP | 32 |
| peptide 3 | KGFSLSDVPQAEISGEHL | 33 |
| peptide 4 | QAEISGEHLRICPQGYTC | 34 |
| peptide 5 | RICPQGYTCCTSEMEENL | 35 |
| peptide 6 | CTSEMEENLANRSHAELE | 36 |
| peptide 7 | ANRSHAELETALRDSSRV | 37 |
| peptide 8 | TALRDSSRVLQAMLATQL | 38 |
| peptide 9 | LQAMLATQLRSFDDHFQH | 39 |
| peptide 10 | RSFDDHFQHLLNDSERTL | 40 |
| peptide 11 | LLNDSERTLQATFPGAFG | 41 |
| peptide 12 | QATFPGAFGELYTQNARA | 42 |
| peptide 13 | ELYTQNARAFRDLYSELR | 43 |
| peptide 14 | FRDLYSELRLYYRGANLH | 44 |
| peptide 15 | LYYRGANLHLEETLAEFW | 45 |
| peptide 16 | LEETLAEFWARLLERLFK | 46 |
| peptide 17 | ARLLERLFKQLHPQLLLP | 47 |
| peptide 18 | QLHPQLLLPDDYLDCLGK | 48 |
| peptide 19 | DDYLDCLGKQAEALRPFG | 49 |
| peptide 20 | QAEALRPFGEAPRELRLR | 50 |
| peptide 21 | EAPRELRLRATRAFVAAR | 51 |
| peptide 22 | ATRAFVAARSFVQGLGVA | 52 |
| peptide 23 | SFVQGLGVASDVVRKVAQ | 53 |
| peptide 24 | SDVVRKVAQVPLGPECSR | 54 |
| peptide 25 | VPLGPECSRAVMKLVYCA | 55 |
| peptide 26 | AVMKLVYCAHCLGVPGAR | 56 |
| peptide 27 | HCLGVPGARPCPDYCRNV | 57 |
| peptide 28 | PCPDYCRNVLKGCLANQA | 58 |
| peptide 29 | LKGCLANQADLDAEWRNL | 59 |
| peptide 30 | DLDAEWRNLLDSMVLITD | 60 |
| peptide 31 | LDSMVLITDKFWGTSGVE | 61 |
| peptide 32 | KFWGTSGVESVIGSVHTW | 62 |
| peptide 33 | SVIGSVHTWLAEAINALQ | 63 |
| peptide 34 | LAEAINALQDNRDTLTAK | 64 |
| peptide 35 | DNRDTLTAKVIQGCGNPK | 65 |
| peptide 36 | VIQGCGNPKVNPQGPGPE | 66 |
| peptide 37 | VNPQGPGPEEKRRRGKLA | 67 |
| peptide 38 | EKRRRGKLAPRERPPSGT | 68 |
| peptide 39 | PRERPPSGTLEKLVSEAK | 69 |
| peptide 40 | LEKLVSEAKAQLRDVQDF | 70 |
| peptide 41 | AQLRDVQDFWISLPGTLC | 71 |
| peptide 42 | WISLPGTLCSEKMALSTA | 72 |
| peptide 43 | SEKMALSTASDDRCWNGM | 73 |
| peptide 44 | SDDRCWNGMARGRYLPEV | 74 |
| peptide 45 | ARGRYLPEVMGDGLANQI | 75 |
| peptide 46 | MGDGLANQINNPEVEVDI | 76 |
| peptide 47 | NNPEVEVDITKPDMTIRQ | 77 |
| peptide 48 | TKPDMTIRQQIMQLKIMT | 78 |
| peptide 49 | QIMQLKIMTNRLRSAYNG | 79 |
| peptide 50 | NRLRSAYNGNDVDFQDAS | 80 |
| peptide 51 | NDVDFQDASDDGSGSGSG | 81 |
| peptide 52 | DDGSGSGSGDGCLDDLCS | 82 |
| peptide 53 | DGCLDDLCSRKVSRKSSS | 83 |
| peptide 54 | RKVSRKSSSSRTPLTHAL | 84 |
| peptide 55 | SRTPLTHALPGLSEQEGQ | 85 |
| peptide 56 | PGLSEQEGQKTSAAS | 86 |

### GPC1 Expression Is Elevated in Pancreatic Cancer

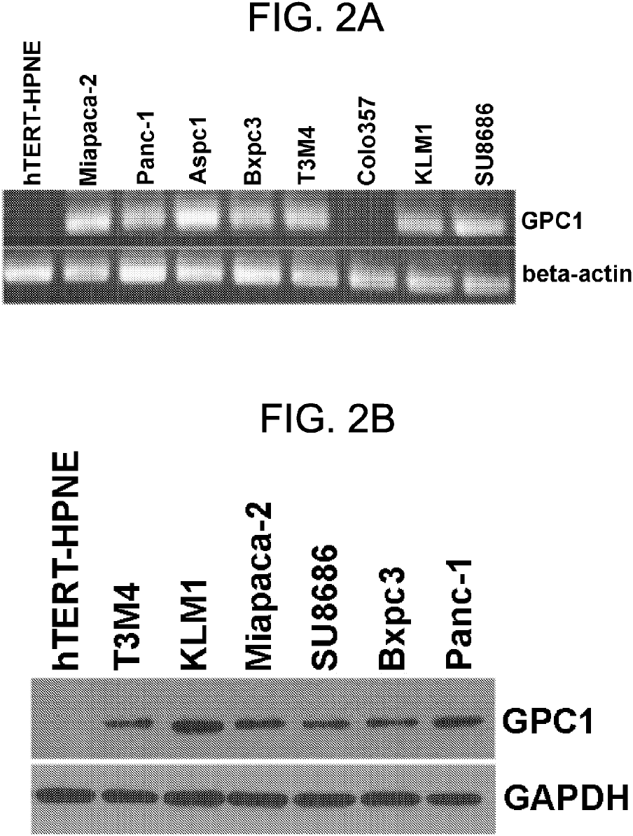

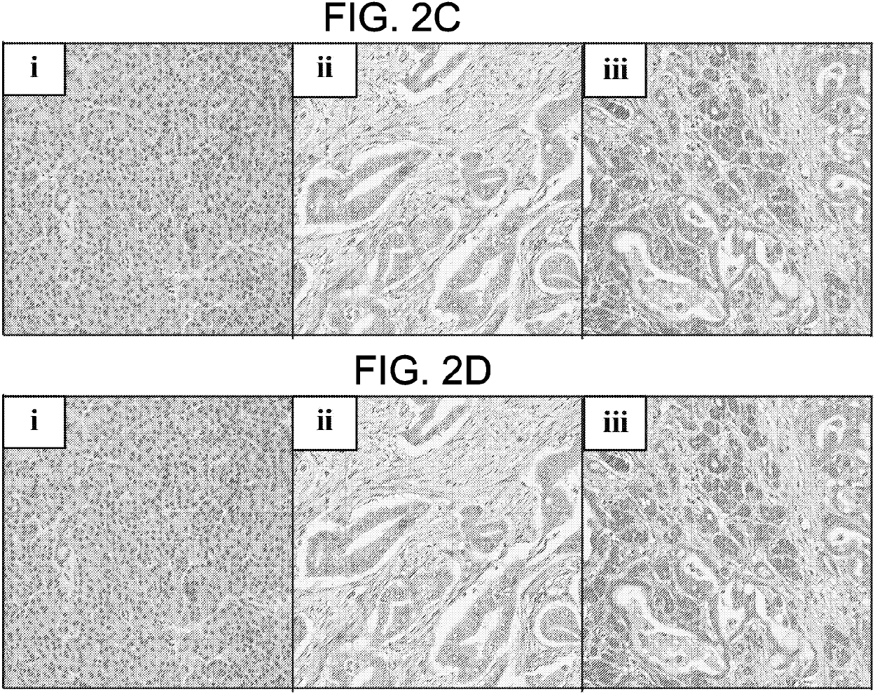

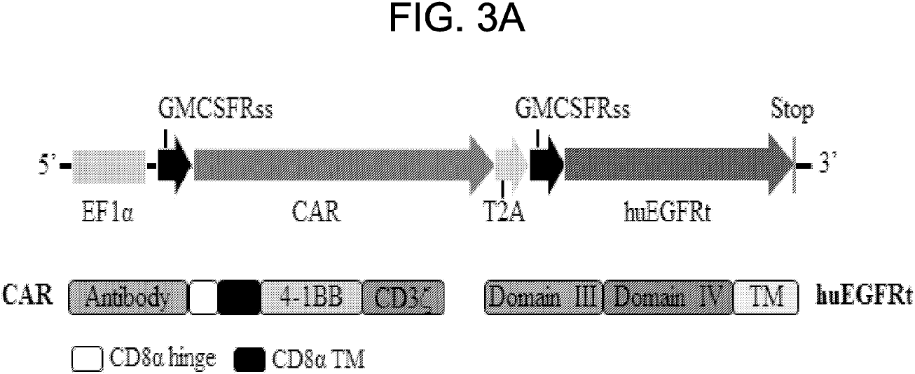

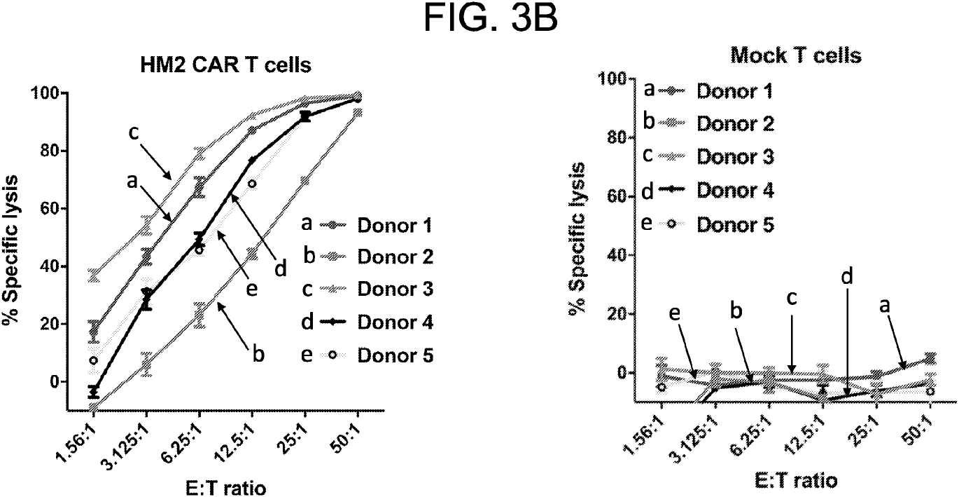

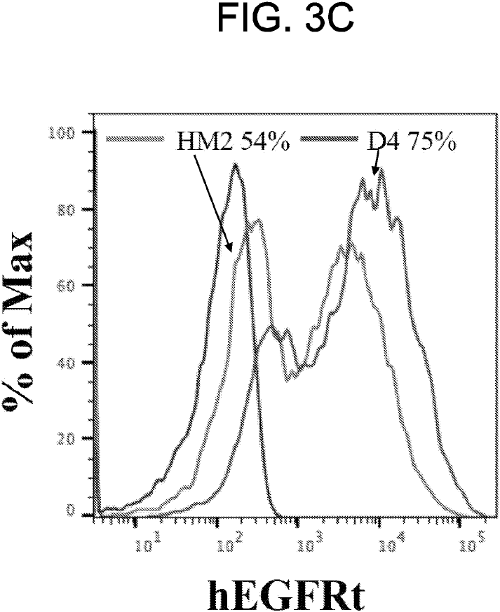

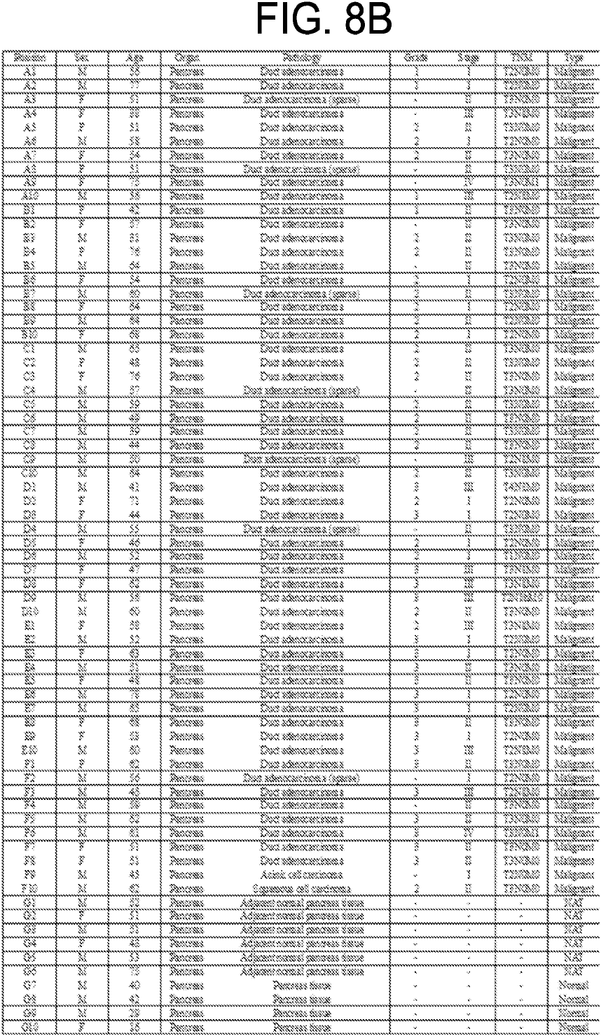

To assess GPC1 levels in pancreatic cancer, RT-PCR and western blot were performed using a panel of pancreatic cancer cell lines and a normal human pancreatic duct epithelial cell line (hTERT-HPNE). GPC1 mRNA and protein levels were appreciably higher in nearly 90% of pancreatic cancer cell lines compared with normal pancreatic duct epithelial cells (FIGS. 2A and 2B). Next, the altered GPC1 expression in pancreatic cancer was evaluated by performing IHC with the HM2 antibody. As shown in FIG. 2C, elevated GPC1 expression was found in pancreatic tumor tissues from low-intermediate (ii) to high levels (iii), but GPC1 labeling was absent in normal pancreas (i). Moreover, GPC1 expression was detected in fibroblasts surrounding the cancer cells, which is consistent with previous reports (HIeeff et al., J Clin Invest 1998;102:1662-1673). Among 60 specimens of pancreatic cancer, 11 cases (18.3%) showed strong GPC1 immunostaining, 41 cases (68.3%) showed low to intermediate levels of staining, and no immunoreactivity was observed in 8 cases (13.3%) (FIGS. 8A-8B). Normal tissue adjacent to the tumor (referred to as NAT) is an intermediate and pre-neoplastic state between healthy and tumor tissue (Aran et al., Nat Commun 2017;8:1077). GPC1 expression was increased in 4 of 6 NAT specimens (FIG. 2D and FIGS. 8A-8B), indicating GPC1 could play a role in pancreatic cancer tumorigenesis and/or progression. Thus, both tumor and local NAT (stroma) could be recognized by GPC1-targeted therapeutics, thereby improving efficacy.

### GPC1-Targeted CAR T Cells Specifically Kill GPC1-Positive Tumor Cells

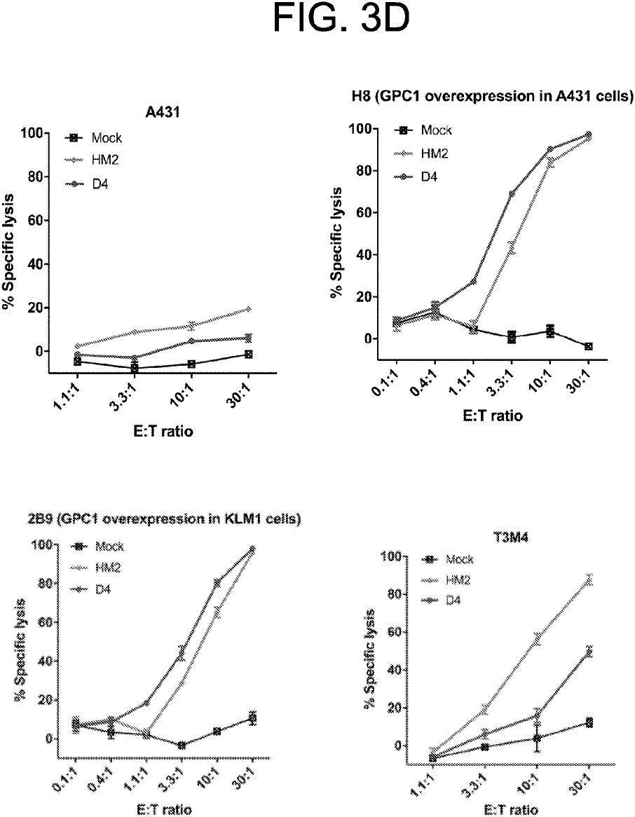

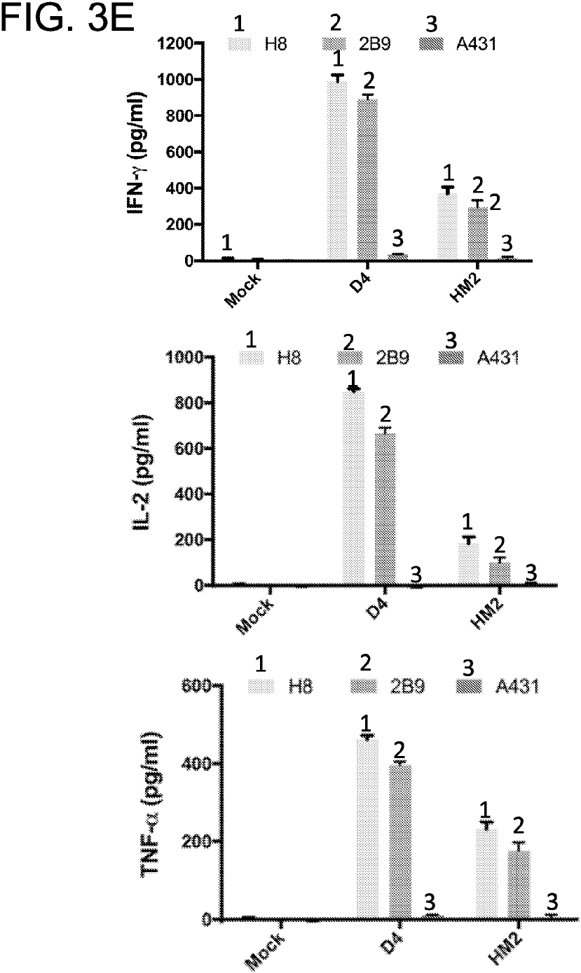

To evaluate the therapeutic value of HM2 and D4 antibodies, CARs were generated that included HM2 or D4 variable fragment, the hinge and TM domains from CD8, and a 4-1BB endodomain (FIG. 3A). To produce CAR T cells from a desired donor, the killing ability of HM2 CAR T cells generated from five healthy donors were tested. As shown in FIG. 3B, HM2 CAR T cells lysed 23% to 79% of the 2B9 tumor cells at an effector:target (E:T) of 6.25:1. By contrast, minimal cell lysis was observed in the 2B9 cells treated with mock T cells. Donor 3 showed the best cytolytic activity among all five donors, and was therefore chosen for comparing the HM2 and D4 CARs in GPC1-positive cells and animal models. As shown in FIG. 3C, the transduction efficiency of activated HM2 and D4 CAR T cells was 54% and 75%, respectively. To compare the cytolytic capacity of HM2 CAR and D4 CAR, CAR T cells were co-cultured with GPC1-negative A431 and GPC1-positive tumor cell lines H8, 2B9 and T3M4. Both H8 and 2B9 cells were effectively lysed by HM2 and D4 CAR T cells even at low E:T ratios with similar potency (FIG. 3D). Minimal cell lysis was observed in A431 cells treated with GPC1-targeted CAR T cells, demonstrating target-dependent specificity. At the high E:T ratio of 30:1, HM2 CAR T cells and D4 CAR T cells killed 88% and 50% of T3M4 cells, which express a low level of GPC1. Although both HM2 and D4 CAR T cells exhibited similar killing ability, D4 CAR T cells triggered 2- to 7-fold more secretion of cytokines including INF-γ, IL-2, and TNF-α than HM2 CAR T cells after exposure to GPC1-positive tumor cells (FIG. 3E). Taken together, HM2 and D4 CAR T cells show comparable selective cytotoxicity against GPC1-positive tumor cells.

### GPC1-Targeted CAR T Cells Suppress the Growth of Pancreatic Cancer Xenografts in Mice

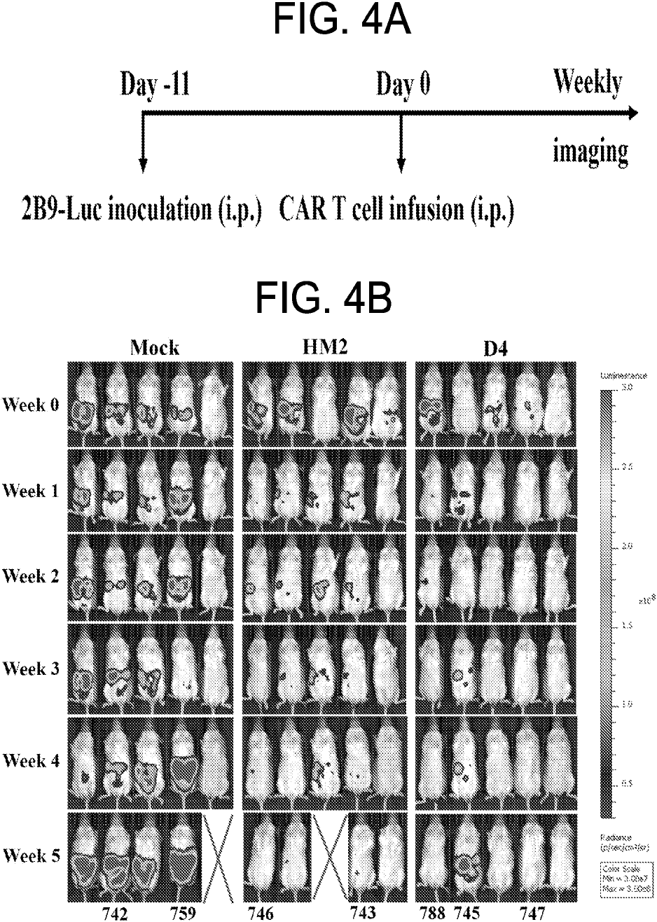

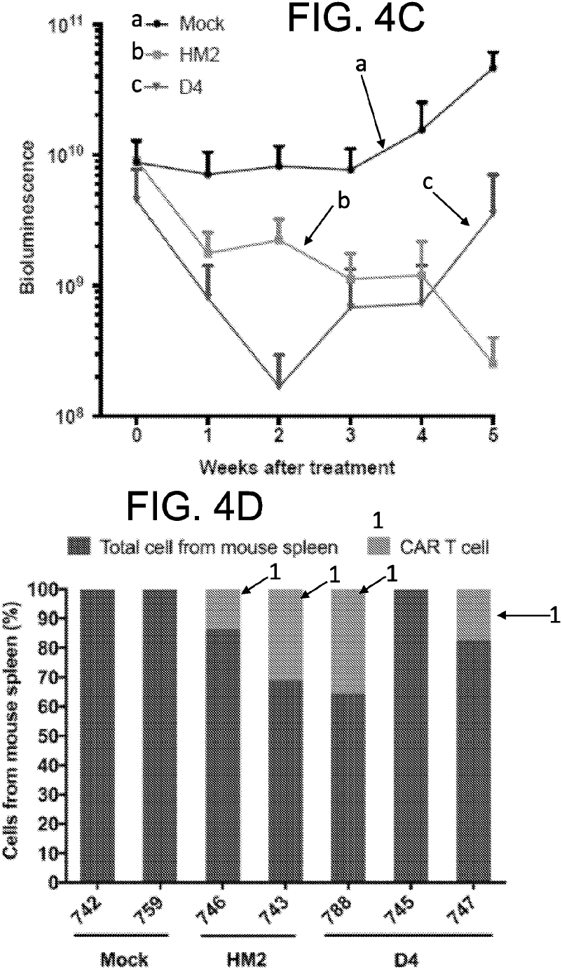

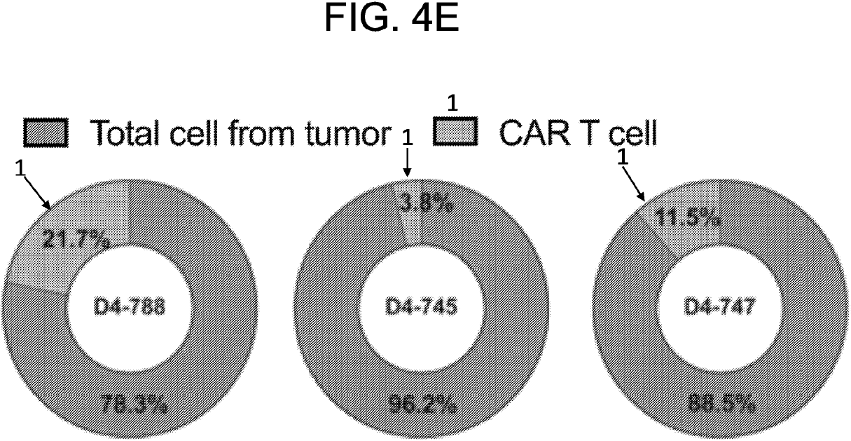

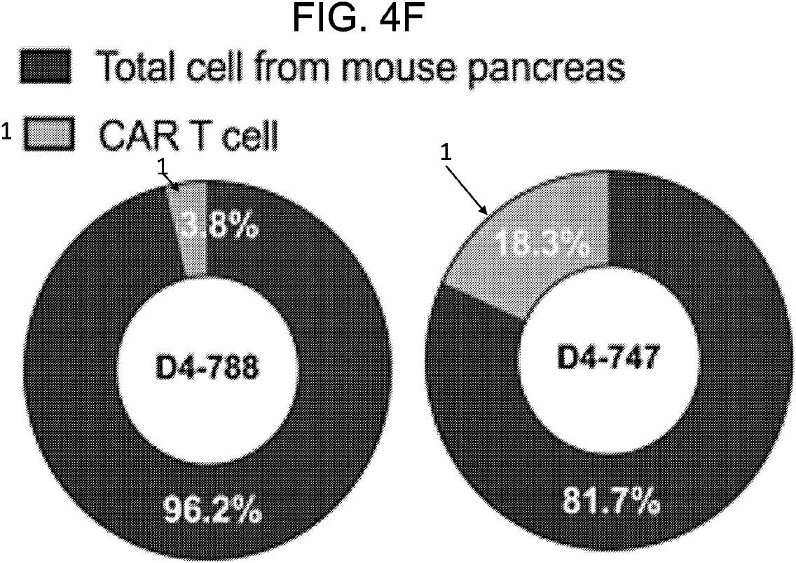

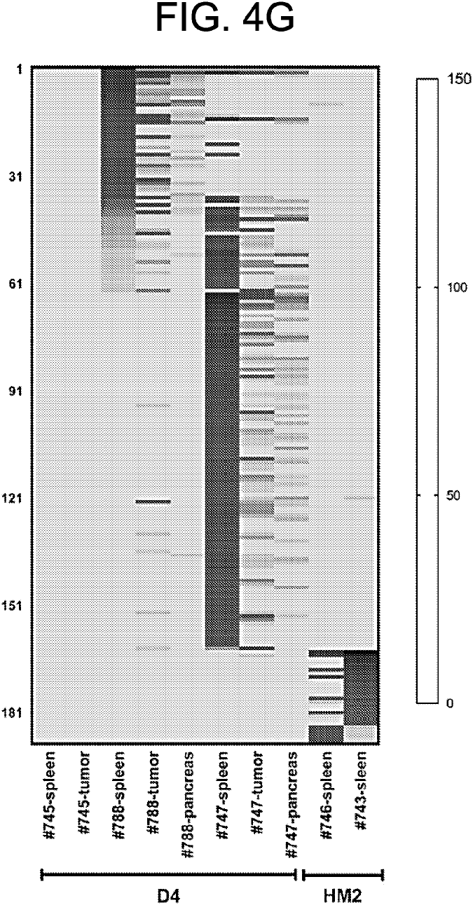

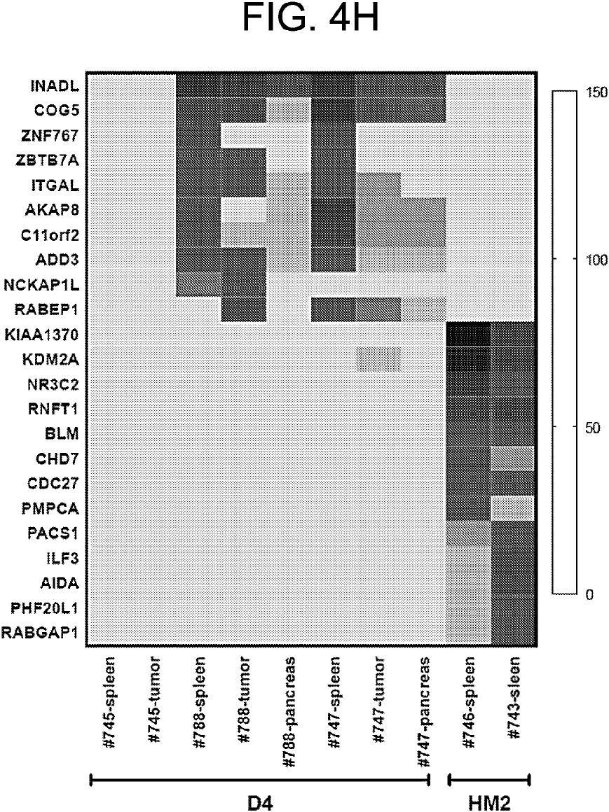

To evaluate the antitumor activities of GPC1-specific CAR T cells in vivo, NSG mice were intraperitoneally (i.p.) injected with 2B9-Luc cells. A single infusion of 10 million mock or CAR T cells was administered i.p. 11 days post inoculation (FIG. 4A). Both HM2 and D4 groups showed reduced tumor burden compared with the mock T cell-treated group (FIGS. 4B and 4C). 80% of NSG mice that received either HM2 or D4 CAR T cells were alive without recurrence by week 5 post-infusion. Robust in vivo expansion and survival of genetically modified T cells are also considered critical predictors of durable clinical remissions in cancer patients. The percentage of CAR T cells were assessed using ddPCR, which allows measurement of absolute gene copy number to determine CAR vector-positive cells. As shown in FIG. 4D, 13.9%-35.7% of CAR vector-positive cells were found in the spleen from responders of HM2 and D4 CAR T cell treatment, whereas no CAR vector-positive cells were detected in the non-responder (#745) in the D4 CAR group, demonstrating an inverse correlation between tumor burden and T cell persistence. Consistently, among three mice receiving D4 CAR T cells, a 3.0-fold to 5.7-fold increase of CAR vector-positive cells were observed in tumor tissues from two responders compared with the non-responder (FIG. 4E). In addition, CAR vector-positive cells were detected in pancreas from two responders in the D4 CAR group (FIG. 4F).

To further understand the molecular determinants of GPC1-targeted CAR T cell efficacy and persistence, the lentiviral integration sites of HM2 and D4 CAR T cells recovered from the spleen, tumor and pancreas of mice at week 5 post-treatment were analyzed. As shown in FIG. 4G, HM2 CAR and D4 CAR showed a strong integration preference into distinct genes. Integration sites were identified in clusters of genes from two responders to D4 CAR T cell treatment, whereas no integration site was found in the non-responder of the D4 CAR group. Notably, the integration sites were largely shared between different tissues (e.g., spleen, tumor and pancreas) of the same mouse, indicating clonal expansion of CAR T cells in mice. Ten and thirteen shared integrated genes were identified in responders to D4 CAR T cells and HM2 CAR T cells, respectively (FIG. 4H). Taken together, both HM2 CAR T cells and D4 CAR T cells persisted and regressed high GPC1-expressing xenograft tumors in mice.

### D4 CAR With IgG4 Hinge and CD28 TM Domain Demonstrate Enhanced Reactivity Against Low-GPC1-Expressing Tumor Cells

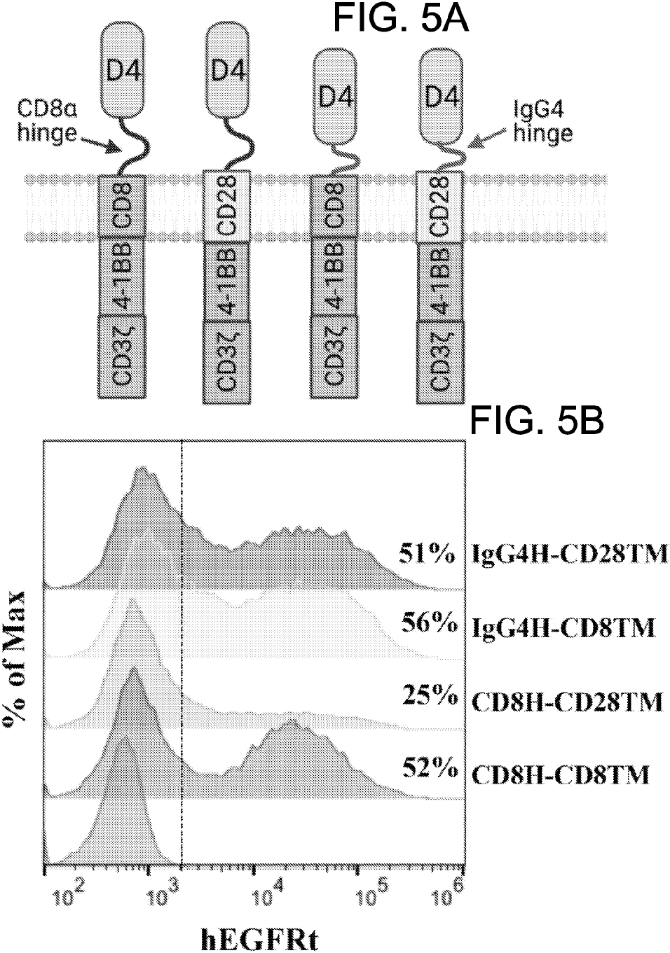

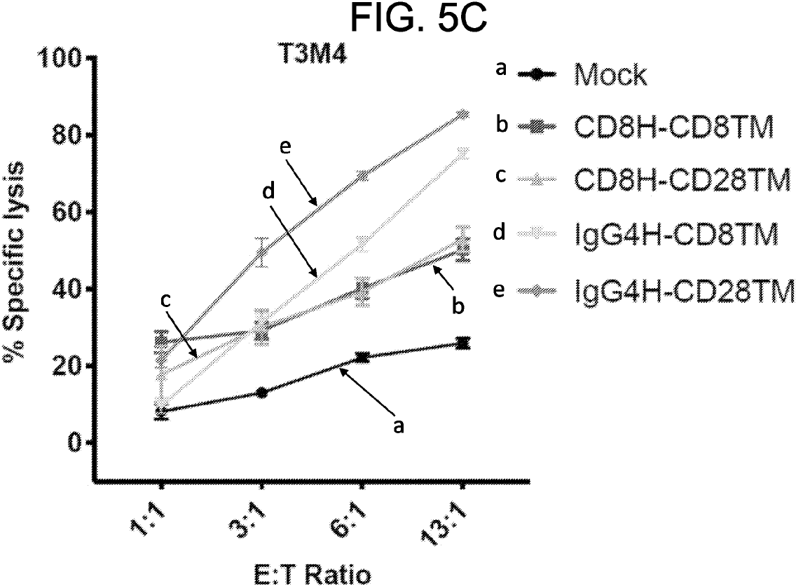

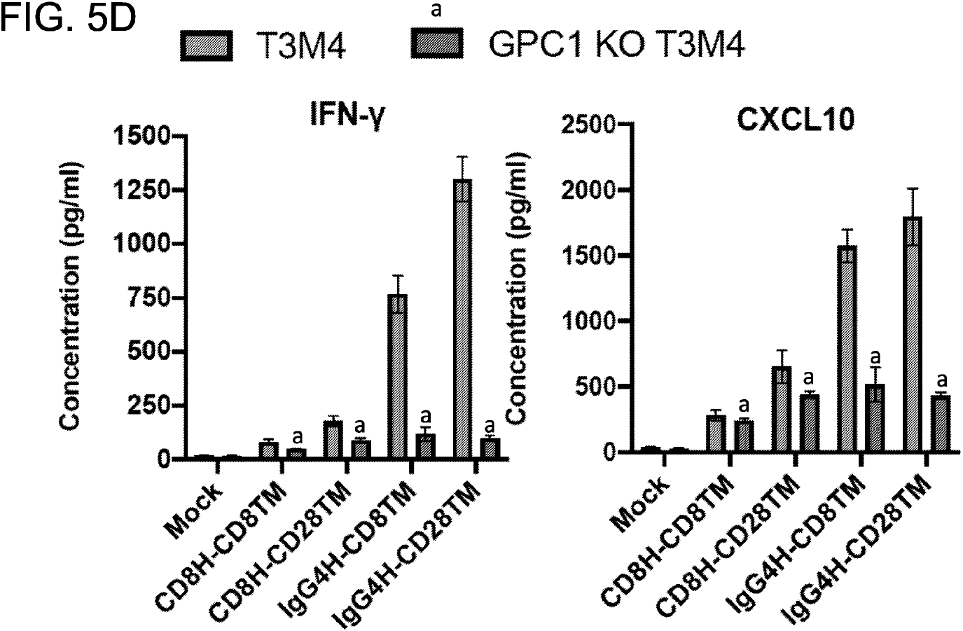

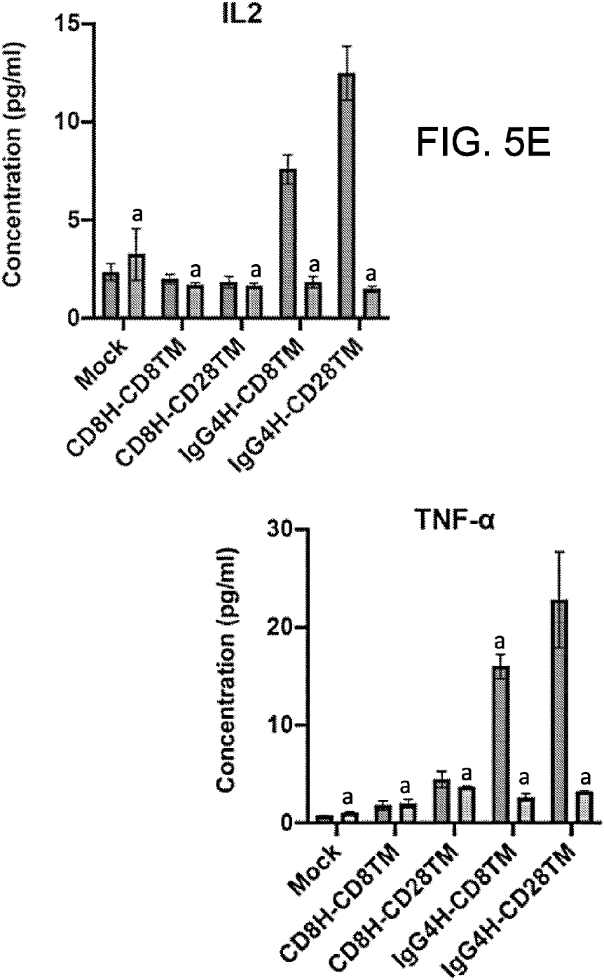

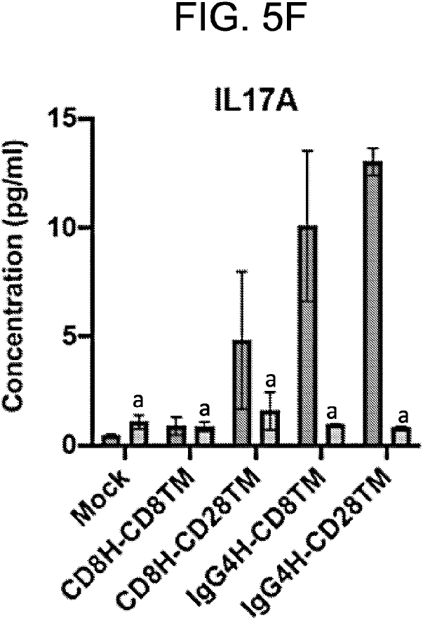

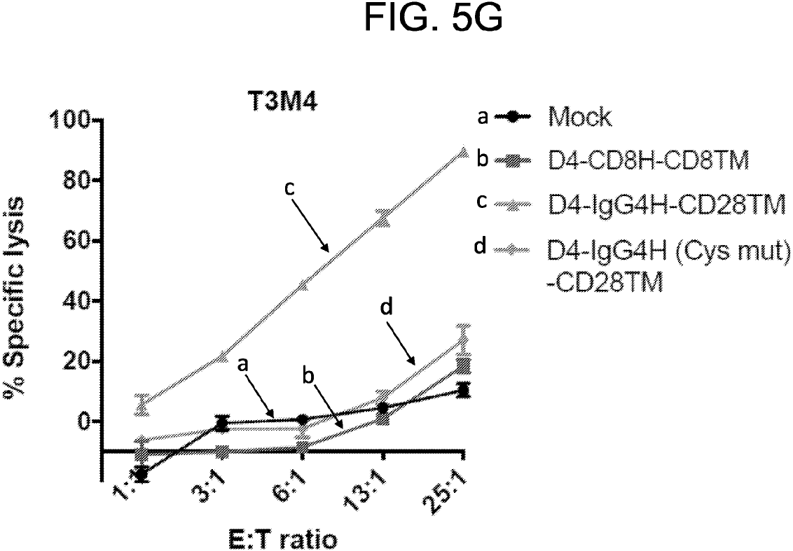

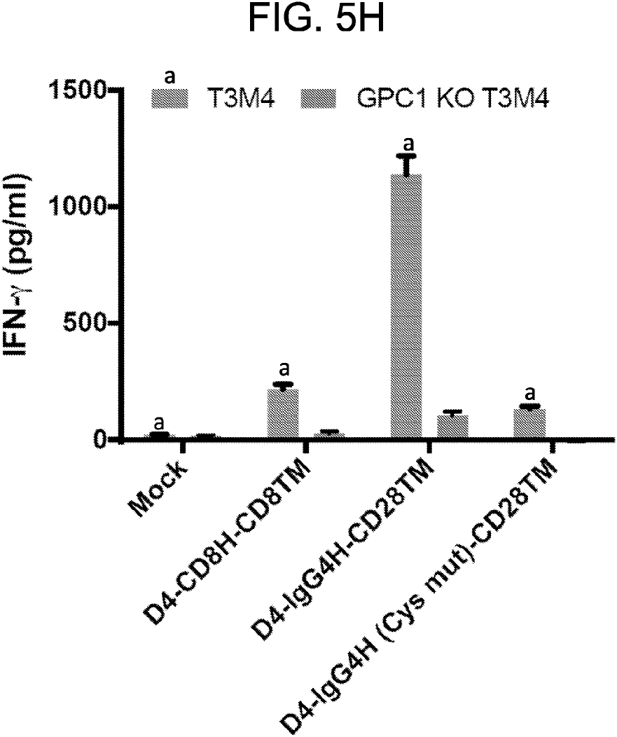

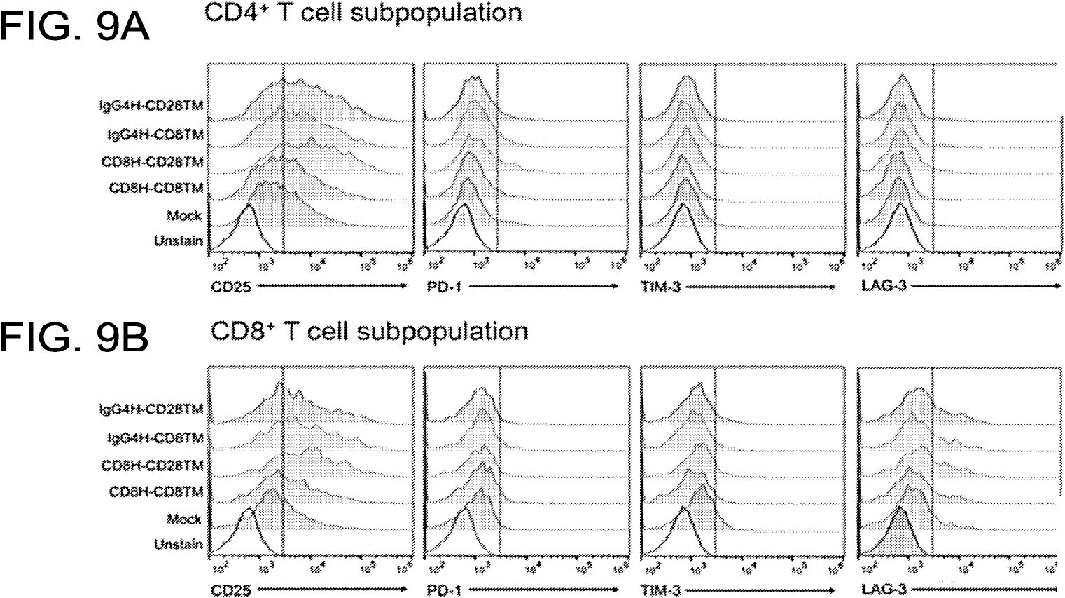

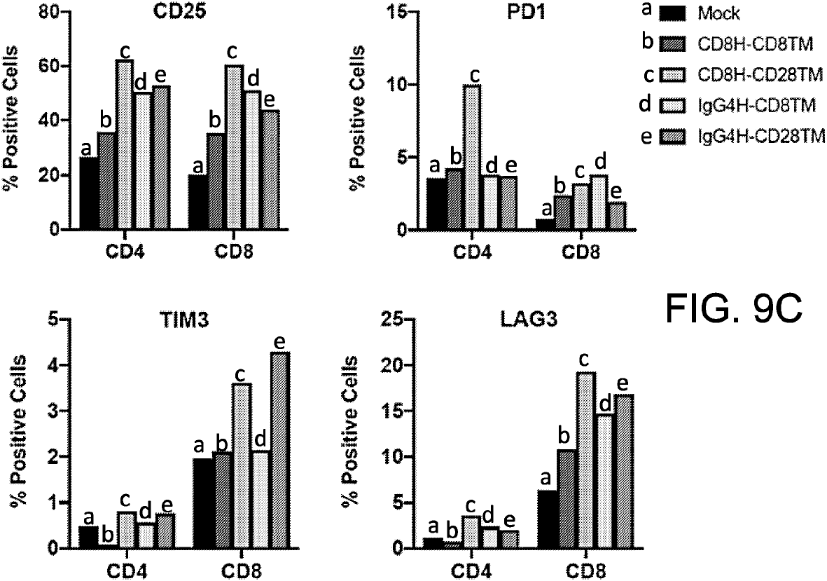

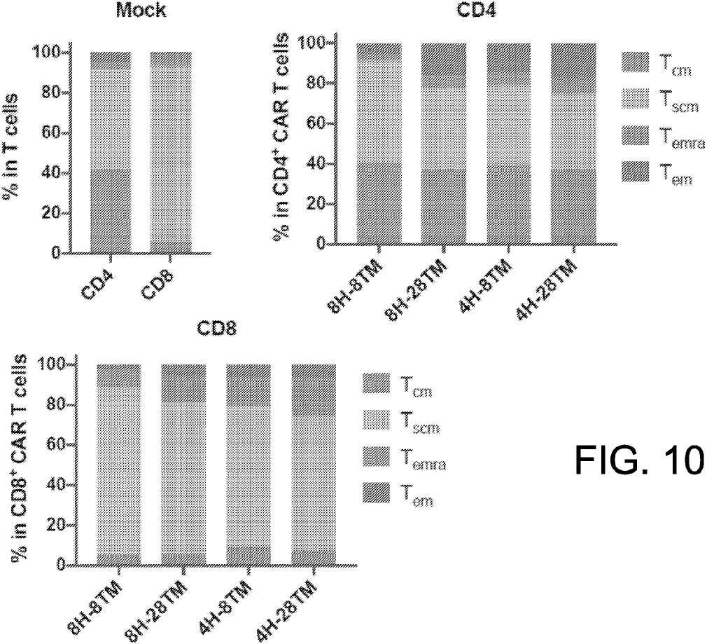

As both HM2 and D4 CAR T cells only killed low GPC1-expressing T3M4 tumor cells at high E:T ratios and D4 CAR T cells were able to produce higher levels of cytokines compared with HM2 CAR T cells, the D4 CAR construct was engineered to improve its reactivity against low-GPC1-expressing cells. The hinge provides flexibility to access the targeted antigen. A previous study demonstrated that the optimal spacer length of a given CAR depends on the position of the targeted epitope (Guest et al., J Immunother 2005;28:203-211). Since D4 recognizes a N-lobe epitope on GPC1, it was hypothesized that shortening the spacer domain might improve T cell signaling. Therefore, a 45-aa CD8 hinge in the initial D4 CAR construct was replaced with a 12-aa IgG4 hinge (FIG. 5A and Table 11). The CD8 TM domain was also compared with the CD28 TM domain that is commonly incorporated along with the IgG4 hinge. Surface expression of each of the CARs was confirmed by staining with the anti-EGFR antibody cetuximab (>80% transduction efficiency) (FIG. 5B). First, the effect of hinge and TM on tonic signaling during ex vivo expansion was examined. D4-CD8 hinge-CD28TM CAR T cells showed appreciably higher levels of T cell activation (CD25) and exhaustion markers (e.g., PD1) than other constructs (FIGS. 9A-9C). T differentiation subsets consisting of stem cell-like memory T cells (T_{SCM}: CD62L+CD45RA+CD95+), central memory T cells (T_{CM}: CD62L+CD45RA-CD95+), effector memory T cells (T_{EM}: CD62L-CD45RA-CD95+) and terminally differentiated effector memory T cells (T_{EMRA}: CD62L-CD45RA+CD95+) were also analyzed. All three engineered D4 CARs increased the frequencies of T_{EM} in the CD4+ T cell population and T_{EMRA} in the CD8+ T cell population compared with the original D4-CD8 hinge-CD8TM CAR (FIG. 10), indicating the IgG4 hinge and/or CD28TM promote the CAR T cell differentiation.

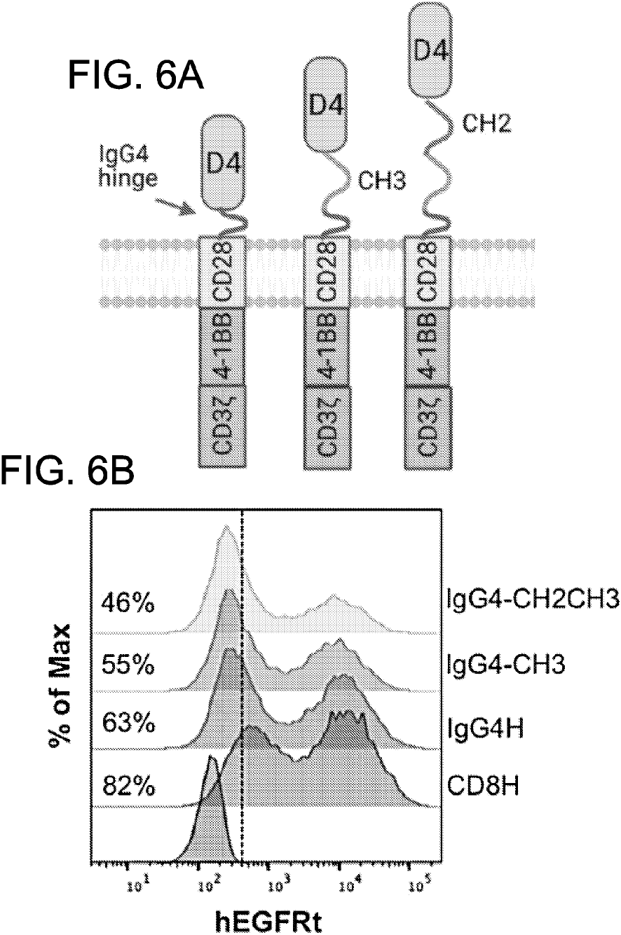

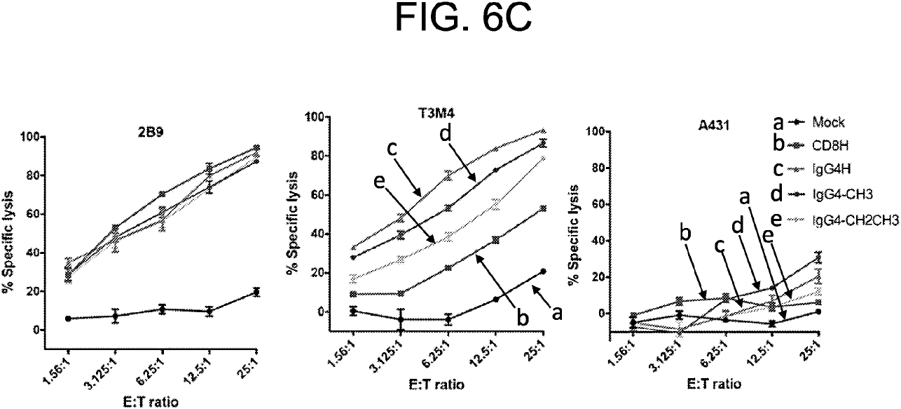

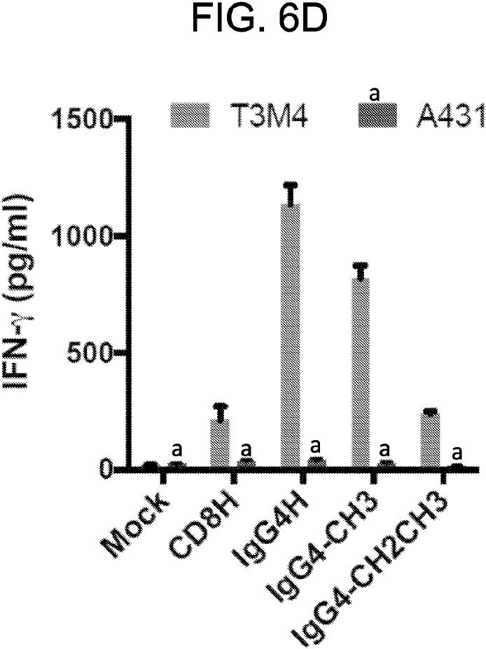

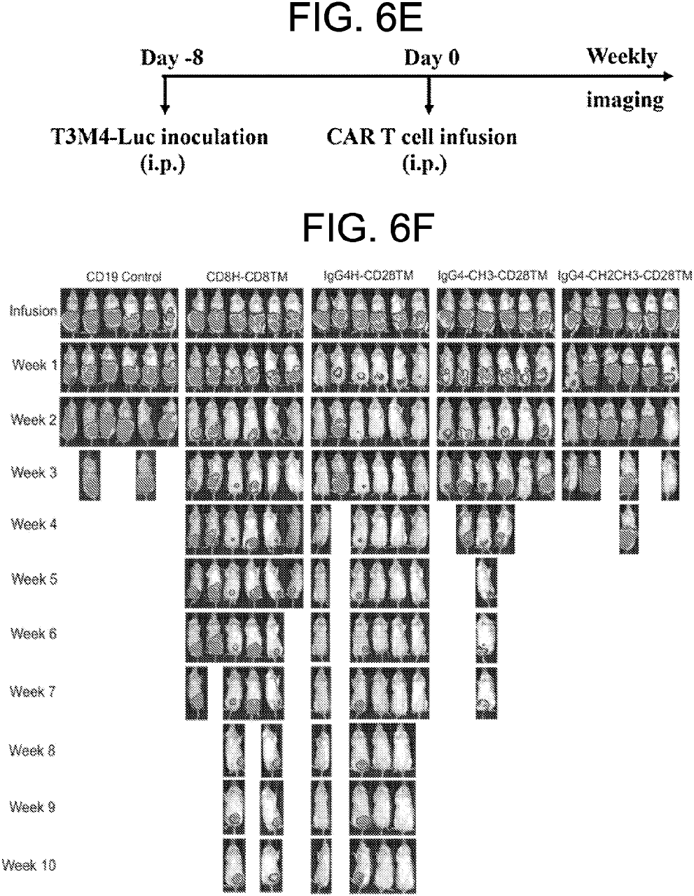

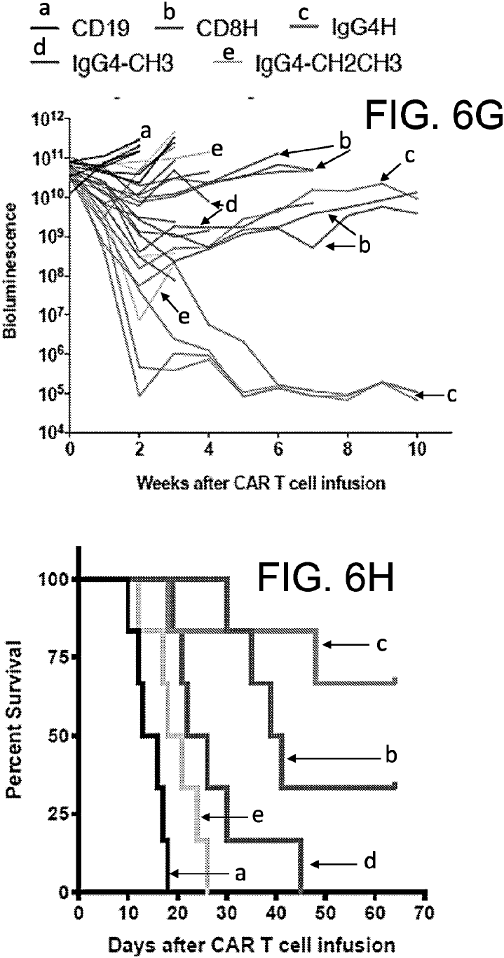

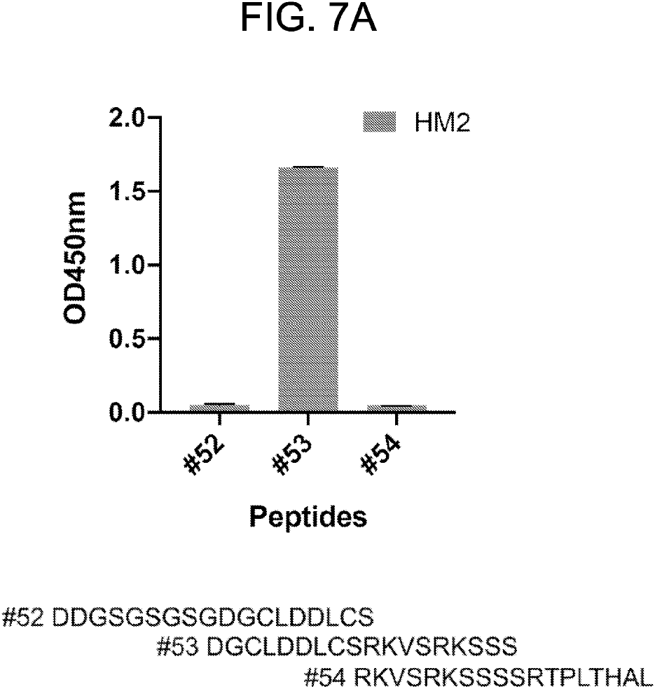

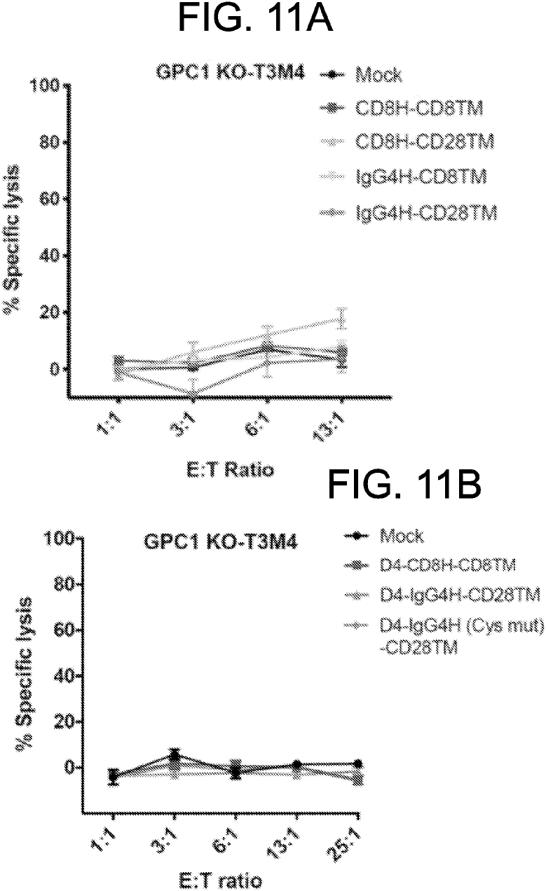

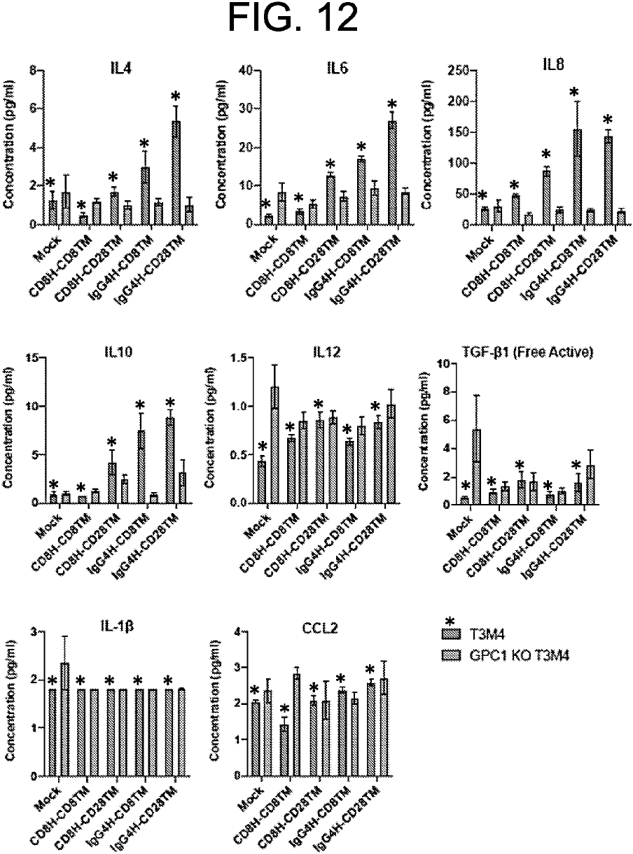

Following exposure to T3M4 cells, the D4-IgG4 hinge-based CAR T cells showed significantly increased cytolytic activity against T3M4 cells compared with the initial D4-CD8 hinge-CAR T cells (FIG. 5C). Particularly, the cytolytic activity of D4-IgG4 hinge-CD28 TM CAR T cells was approximately 10% higher than the D4-IgG4 hinge-CD8 TM CAR T cells against T3M4 cells. However, replacement of CD8TM with CD28TM in the D4-CD8 hinge CAR T cells didn’t improve cell killing ability. None of the four D4 CAR T cells lysed GPC1 knockout T3M4 cells (FIG. 11A), demonstrating target-dependent specificity. Consistent with cell killing potency, D4-IgG4 hinge-CD28TM CAR T cells induced the most secretion of IFN-γ, CXCL10, IL-2, TNF-α, IL-17A, IL-4, IL-6, IL-8 and IL-10 upon stimulation with GPC1-positive T3M4 cells (FIGS. 5D-5F and 12). No difference was seen in secretion of IL-12, TGF-β1 (free active), IL-1β and CCL-2 among different D4 CAR T cells. Furthermore, two cysteine residues were identified in the IgG4 hinge that may form disulfide dimers to enhance T cell signaling. To test the hypothesis, cysteine-to-serine mutations were introduced in the IgG4 hinge (Table 11) and killing ability was compared to the original IgG4 hinge. The enhanced cytolytic activity and IFN-γ secretion of D4-IgG4 hinge-CD28TM CAR T cells were lost when both cysteine residues were mutated (FIGS. 6G and 6H), indicating the interchain disulfide formation is important for the D4-IgG4 hinge CAR. In addition, minimal cell lysis was observed in GPC1 KO-T3M4 cells (FIG. 11B).

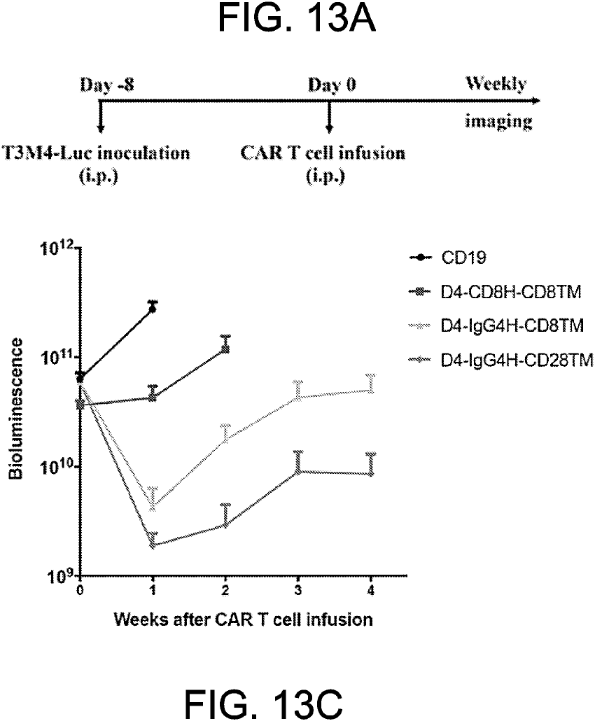

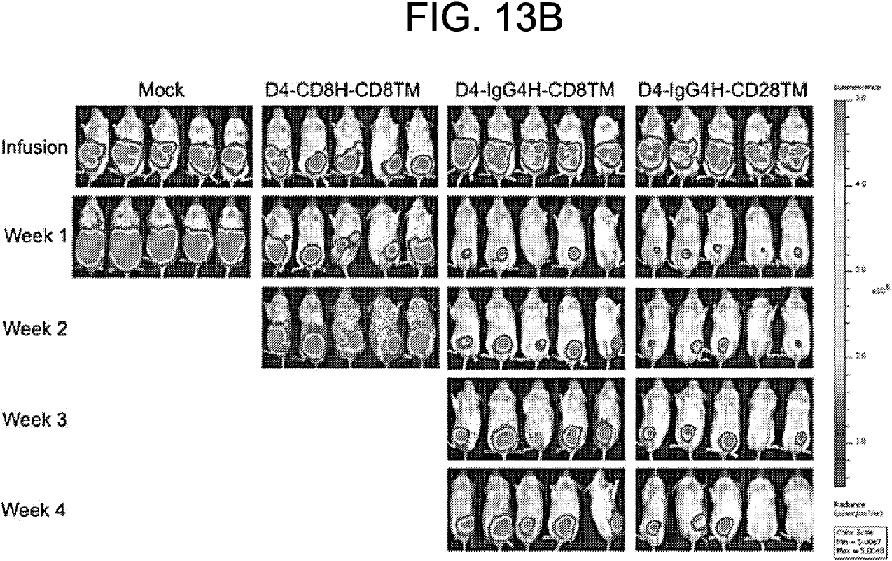

To determine whether the superior in vitro activity of D4-IgG4 hinge-based CAR T cells would translate into improved antitumor activity in vivo, T3M4-Luc cells were i.p. inoculated into NSG mice. A single infusion of 5 million CD19 CAR or one of three formats of D4 CAR T cells were i.p. administered 6 days post-inoculation (FIG. 13A). As shown in FIGS. 13B-13C, mice treated with D4-IgG4 hinge-based CAR T cells had a superior antitumor response and survival compared with mice treated with the initial D4-CD8 hinge CAR T cells. Between the two D4-IgG4 hinge CAR constructs, incorporating a CD28 TM domain outperformed the CD8 TM domain and demonstrated better tumor regression. By contrast, CD19 CAR T cells-treated mice developed large peritoneal tumors necessitating euthanasia 2 weeks after infusion.

TABLE 11

Protein sequences of CD8 hinge and IgG4 hinge w/wo cysteine mutations

| Hinge | Sequence | SEQ ID NO: |
|---|---|---|
| CD8 | TTTPAPRPPTPAPTIASQPLSLRPEACRPAAGGAVHTRGLDFACD | 8 |
| IgG4 | ESKYGPPCPPCP | 7 |
| IgG4-Cys mut | ESKYGPPSPPSP | 30 |

### D4 CAR With a Short IgG4 Hinge Regress Pancreatic Cancer Xenografts in Mice

A CD19 CAR incorporating a longer spacer (IgG4 hinge-CH₂CH₃) with modifications that abrogate binding to Fc receptors showed equivalent antitumor activity to the one with IgG4 hinge only (Hudecek et al., Cancer Immunol Res 2015;3:125-135). To examine this possibility, two additional D4 CARs were constructed in which the modified IgG4-Fc spacer domain was sequentially added to derive D4-IgG4 hinge-CH₃ (intermediate) and D4-IgG4 hinge-CH₂CH₃ (long) variants (FIG. 6A). All three D4-IgG4 hinge-based CARs have a CD28 TM domain. The expression of each of the CARs was confirmed, although the transduction efficiency was slightly decreased as the spacer length increased (FIG. 6B). As shown in FIG. 6C, all three D4-IgG4 hinge-based CAR T cells showed improved reactivity compared with the initial D4-CD8 hinge CAR T cells. T cells expressing the short IgG4 hinge only D4 CAR had maximum cytolytic activity, and a hierarchy (short > intermediate >> long) of tumor lysis was clearly evident against T3M4 cells. By contrast, T cells expressing any of the D4-IgG4 hinge-based CARs and the D4-CD8 hinge-based CAR killed high GPC1-expressing 2B9 cells equally well. Moreover, none of the D4 CAR T cells killed A431 cells. As observed in the cytolytic assay, the short spacer construct was superior in mediating IFN-γ secretion after recognition against T3M4 cells (FIG. 6D).

The antitumor activity of CAR T cells was then compared with different length of spacers using the T3M4 i.p. xenograft mouse model (FIG. 6E). As shown in FIGS. 6F and 6G, mice treated with 10 million T cells expressing D4 CAR with a short spacer had rapid and complete tumor regression within 2 weeks of treatment. The same dose of D4 CAR T cells expressing either the intermediate or the long spacer was less effective in eliminating tumor cells in mice. D4-IgG4-hinge-CD28TM CAR T cells dramatically extended the survival of mice bearing T3M4 xenografts (FIG. 6H). Together, the D4-IgG4 hinge-CD28 TM CAR T cells demonstrate significantly improved antitumor efficacy in pancreatic cancer cells with low GPC1 antigen density.

### Discussion

In the present example, antibodies HM2 and D4 were developed specifically for binding a membrane-proximal C-lobe epitope and a membrane-distal N-lobe epitope of GPC1, and CAR T cells were made to analyze their antitumor activities. HM2 and D4 CAR T regressed high GPC1-expressing tumor growth equally well. The hinge and TM domain of the D4 CAR was also optimized, which significantly improved its efficacy in mice carrying low GPC1-expressing xenograft tumors.

NAT presents a unique intermediate state between healthy and tumor tissues (Aran et al., Nat Commun 2017;8:1077). Cancer cells interact with their immediate and local environment, more specifically, the adjacent stroma. The data disclosed herein demonstrate GPC1 expression is not only increased in pancreatic tumor tissues but is also strongly elevated in NATs compared with normal pancreas, indicating both tumor cells and stroma cells could be recognized by GPC1-specific CAR T cells, which may improve the antitumor activity.

The D4 CAR construct was modified by replacing a 45-aa CD8 hinge with a 12-aa modified IgG4 hinge. As a result, the D4-IgG4 hinge-based CAR T cells had significantly improved antitumor activity compared with D4-CD8 hinge-based CAR T cells against low-GPC1-expressing T3M4 cells. In addition, the D4-IgG4 hinge-CD28 TM CAR T cells had appreciably higher killing activity than D4-IgG4 hinge-CD8 TM CAR T cells. Furthermore, the killing ability (91.6%) of D4-IgG4 hinge-CD28 TM CAR T cells at high E:T ratio was comparable to the killing ability (87.7%) of HM2 CAR T cells targeting the membrane-proximal epitope on GPC1. The results disclosed herein indicate that the IgG4 spacer improves CAR T cell targeting of membrane-distal sites.

Collectively, D4 and HM2 CAR T cells targeting the N-lobe and C-lobe of GPC1, respectively, were generated and their efficacy in a xenograft mouse model was demonstrated. By optimizing the spacer for the D4 CAR, the CAR T cell reactivity against low GPC1-expressing pancreatic cancer cells in vitro and in vivo was significantly improved, which provides clinical applications in GPC1-positive cancers.

In view of the many possible embodiments to which the principles of the disclosed subject matter may be applied, it should be recognized that the illustrated embodiments are only examples of the disclosure and should not be taken as limiting the scope of the disclosure. Rather, the scope of the disclosure is defined by the following claims. We therefore claim all that comes within the scope and spirit of these claims.

1. A chimeric antigen receptor (CAR), comprising:
an extracellular antigen-binding domain that specifically binds glypican-1 (GPC1);
a hinge region consisting of the IgG4 hinge region set forth as SEQ ID NO: 7;
a transmembrane domain;
an intracellular co-stimulatory domain; and
an intracellular signaling domain.

2. The CAR of claim 1, wherein the antigen-binding domain comprises a GPC1-specific single-domain antibody.

3. The CAR of claim 2, wherein the single-domain antibody comprises the complementarity determining region 1 (CDR 1), CDR2 and CDR3 sequences of SEQ ID NO: 6.

4. The CAR of claim 3, wherein the CDR1, CDR2 and CDR3 sequences respectively comprise:
residues 31-35, 50-66 and 99-109 of SEQ ID NO: 6;
residues 26-33, 51-58 and 97-108 of SEQ ID NO: 6;
residues 27-33, 47-61 and 97-108 of SEQ ID NO: 6; or
residues 26-35, 47-66 and 97-108 of SEQ ID NO: 6.

5. The CAR of claim 3, wherein the amino acid sequence of the single-domain antibody is at least 90% identical to SEQ ID NO: 6 and comprises the CDR1, CDR2 and CDR3 sequences of SEQ ID NO: 6.

6. The CAR of claim 3, wherein the amino acid sequence of the single-domain antibody comprises or consists of SEQ ID NO: 6.

7. The CAR of claim 1, wherein the antigen-binding domain comprises a GPC1-specific scFv.

8. The CAR of claim 7, wherein the scFv comprises a variable heavy (VH) domain and a variable light (VL) domain and the VH domain comprises the complementarity determining region 1 (CDR1), CDR2 and CDR3 sequences of SEQ ID NO: 2, and the VL domain comprises the CDR1, CDR2 and CDR3 sequences of SEQ ID NO: 4.

9. The CAR of claim 8, wherein the VH domain CDR1, CDR2 and CDR3 sequences respectively comprise:
residues 31-35, 50-66 and 99-103 of SEQ ID NO: 2;
residues 26-33, 51-58 and 97-103 of SEQ ID NO: 2;
residues 27-35, 47-61 and 97-103 of SEQ ID NO: 2; or
residues 26-35, 47-66 and 97-103 of SEQ ID NO: 2.

10. The CAR of claim 8, wherein the VL domain CDR1, CDR2 and CDR3 sequences respectively comprise:
residues 24-39, 55-61 and 94-102 of SEQ ID NO: 4;
residues 27-37, 55-57 and 94-101 of SEQ ID NO: 4;
residues 28-39, 51-61 and 94-102 of SEQ ID NO: 4; or
residues 24-39, 51-61 and 94-102 of SEQ ID NO: 4.

11. The CAR of claim 8, wherein:
the amino acid sequence of the VH domain is at least 90% identical to SEQ ID NO: 2 and comprises the CDR1, CDR2 and CDR3 sequences of SEQ ID NO: 2; and
the amino acid sequence of the VL domain is at least 90% identical to SEQ ID NO: 4 and comprises the CDR1, CDR2 and CDR3 sequences of SEQ ID NO: 4.

12. The CAR of claim 8, wherein:
the amino acid sequence of the VH domain comprises or consists of SEQ ID NO: 2; and
the amino acid sequence of the VL domain comprises or consists of SEQ ID NO: 4.

13. The CAR of claim 8, wherein the scFv comprises the amino acid sequence of residues 25-265 of SEQ ID NO: 18.

14. The CAR of claim 1, wherein the transmembrane domain comprises a CD28 transmembrane domain.

15. The CAR of claim 1, wherein the co-stimulatory domain comprises a 4-1BB signaling moiety.

16. The CAR of claim 1, wherein the signaling domain comprises a CD3ζ signaling domain.

17. An isolated cell expressing the CAR of claim 1.

18. The isolated cell of claim 17, which is a T cell, a natural killer (NK) cell or a macrophage.

19. A nucleic acid molecule encoding the CAR of claim 1.

20. The nucleic acid molecule of claim 19, operably linked to a promoter.

21. The nucleic acid molecule of claim 19, comprising in the 5′ to 3′ direction:
a nucleic acid encoding a first granulocyte-macrophage colony stimulating factor receptor signal sequence (GMCSFRss);
a nucleic acid encoding the antigen-binding domain;
a nucleic acid encoding the IgG4 hinge region;
a nucleic acid encoding the transmembrane domain;
a nucleic acid encoding the co-stimulatory domain;
a nucleic acid encoding the signaling domain;
a nucleic acid encoding a self-cleaving 2A peptide;
a nucleic acid encoding a second GMCSFRss; and
a nucleic acid encoding a truncated human epidermal growth factor receptor (huEGFRt).

22. The nucleic acid molecule of claim 21, further comprising a human elongation factor 1α (EF1α) promoter sequence 5′ of the nucleic acid encoding the first GMCSFRss.

23. A vector comprising the nucleic acid molecule of claim 19.

24. The vector of claim 23, wherein the vector is a lentiviral vector.

25. An isolated cell comprising the nucleic acid molecule of claim 19.

26. The isolated cell of claim 25, which is a T cell, an NK cell or a macrophage.

27. A composition comprising a pharmaceutically acceptable carrier and the cell of claim 17.

28. A method of treating a GPC 1-positive cancer in a subject, comprising administering to the subject a therapeutically effective amount of cell of claim 17.

29. A method of inhibiting tumor growth or metastasis of a GPC1-positive cancer in a subject, comprising administering to the subject a therapeutically effective amount of the cell of claim 17.

30. The method of claim 28, wherein the GPC1-positive cancer is a solid tumor.

31. The method of claim 28, wherein the GPC I-positive cancer is a pancreatic cancer, colorectal cancer, liver cancer, glioma, lung cancer, head and neck cancer, thyroid cancer, osteosarcoma, endometrial cancer, breast cancer or ovarian cancer.

32. The method of claim 28, wherein the GPC I-positive cancer expresses no more than about 2500, molecules of GPC1 per cell.

33. The method of claim 32, wherein the GPC I-positive cancer is a pancreatic cancer.
